(19) United States
(12) Patent Application Publication
(10) Pub. No.: US 2013/0065814 A1
(43) Pub. Date: Mar. 14, 2013

(54) INDUCTIVE PRODUCTION OF PLURIPOTENT STEM CELLS USING SYNTHETIC TRANSCRIPTION FACTORS

(71) Applicants: Guoliang Xu, Shanghai (CN); Yang Wang, Shanghai (CN)

(72) Inventors: Guoliang Xu, Shanghai (CN); Yang Wang, Shanghai (CN)

(73) Assignee: SHANGHAI INSTITUTES FOR BIOLOGICAL SCIENCES, CAS, Shanghai (CN)

(21) Appl. No.: 13/583,829

(22) PCT Filed: Mar. 7, 2011
(86) PCT No.: PCT/CN2011/000360

§ 371 Date: Sep. 10, 2012

(30) Foreign Application Priority Data
Mar. 9, 2010 (CN) 201010119884.4
Jan. 31, 2011 (CN) 201110034261.1

(51) Int. Cl. C12N 5/071; C12N 15/86; A61K 31/7088; C12N 15/62; A61K 38/02; C12N 15/85; C07K 19/00

(52) U.S. Cl. 514/1.1; 435/320.1; 435/377; 435/456; 435/455; 530/350; 536/23.4; 514/44 R

(57) ABSTRACT

The present invention relates to use of synthetic factors in reprogramming somatic cells to become induced pluripotent stem cells and other cell lineages. Specifically, the present application relates to fusion proteins containing proteins encoded by cell totipotency-related genes and transcription regulatory domains, their coding sequences, expression vectors, and compositions. The present application also relates to methods for reprogramming somatic cells to become induced pluripotent stem cells and other cell lineages, and cells containing the fusion proteins or the coding sequences.

### TECHNICAL FIELD

The present invention relates to the field of pluripotent stem cells. Specifically speaking, the present invention relates to using synthetic factors to reprogram somatic cells to become induced pluripotent stem cells or other types of cells.

### BACKGROUND TECHNOLOGY

Embryonic stem cells are derived from inner cell mass of blastocyst stage embryos, capable of undergoing self-renewal and maintaining pluripotency (Evans and Kaufman, 1981; Martin, 1981). In 1998, Thomson successfully established and cultivated human pluripotent stem cell lines (Thomson et al., 1998). Subsequently, a large body of research shows that human embryonic stem cells established by using the Thomson method can self-renew indefinitely in vitro, and can differentiate into cells of almost all human tissue types. One may culture the stem cells in vitro, directionally induce them to differentiate into various desired tissue cells, and then, by various means, introduce these differentiated cells into animals of diease models. Resutls from these experiments show that stem cells can greatly improve disease states in these animals, thereby giving rise to therapy using stem cell transplantation.

Embryonic stem cells not only can provide almost limitless sources of cells for cell transplantation therapy, but also offer the possibility of providing the desired cell types for almost all organs, presenting a bright prospect for tissue engineering and regenerative medicine (Daley and Scadden, 2008). However, sources for human embryonic stem cells, especially patient-specific stem cells, have become a difficult problem puzzling the scientific community, and many researchers have focused their attention on the readily available somatic cells, hoping to make the differentiated somatic cells to undergo reprogramming and regain ES-likepluripotency (Jaenisch and Young, 2008; Yamanaka, 2007).

Before 2006, there were three ways to reprogram somatic cells: reprogramming by nuclear transplant, reprogramming by fusion with ES cells, and spontaneous reprogramming in long-term culturing.

From the success of the first nuclear transplantation in 1952 (Briggs and King, 1952) to the birth of the cloned sheep Dolly in 1997 (Dolly Wilmut et al., 1997), somatic cell cloning technology has gradually matured, and cloning in different species and somatic cell types have subsequently been achieved (Gurdon and Byrne, 2003). Stem cells obtained through nuclear transplantation can effectively solve the problems of immune rejection after cell transplantation. However, the low efficiency of nuclear transplantation, developmental abnormalities of animals obtained by somatic cell cloning, as well as a series of problems such as the ethical controversy related to the sources of human oocytes and the use of human embryos have become bottleneck problems for the development of therapeutic somatic cell cloning (Jaenisch and Young, 2008; Yamanaka, 2007).

It was meanwhile revealed that lymphocytes, after fusion with stem cells, possess pluripotency (Miller and Ruddle, 1976; Tada et al., 2001), and these cells, when injected into nude mice, generated three germ layers of cells. A recent study found that fusion with human ES cells also leads to reprogramming (Cowan et al., 2005; Yuet al., 2006). However, removal of chromosomes that originated from ES cells from the reprogrammed cells is a technical challenge (Jaenisch and Young, 2008; Yamanaka, 2007). Other research groups also attempted to explore the use of ES cell extracts to reprogram somatic cells (Taranger et al., 2005).

Cells from the inner cell mass, after being cultured in vitro, produce embryonic stem cells (ES cells), and the primordial germ cells, after being cultured under in vitro conditions, can produce pluripotent embryonic germ cells (EG cells) (Matsui et al., 1992). Researchers think other types of cells can possibly also produce pluripotent cells under conditions of long-term culture in vitro. Thus far, multipotent adult progenitor cells (MAPCs) have been produced by long-term culture of bone marrow cells in vitro (Jiang et al., 2002), and multipotent adult germline stem cells (maGS) have also been produced from seminal vesicles of adult mice (Guan et al., 2006). These two types of cells each can generate chimeric mice, when injected into blastocysts. However, whether this method is suitable for use in other cell types remains unknown.

The three reprogramming methods mentioned above all have some deficiencies. Therefore, scientists worldwide are actively exploring other reprogramming strategies. In 2006, the research group of Yamanaka at Kyoto University in Japan found, by using ingenious experimental strategies, that, transfer of only 4 transcription factors, Oct4, Sox2, Klf4 and C-Myc, via viral infection into mouse fibroblasts, can make the fibroblasts gain pluripotency similar to that of ES cells. They named such cells “induced pluripotent stem cells” (induced pluripotent stem cells, iPS) (Takahashi and Yamanaka, 2006). However, the efficiency of this method is very low, about 0.01%-0.1%.

Subsequent studies showed that the induced pluripotent stem cells (iPS cells), when injected into blastocyst, could produce chimeric mice, demonstrating a similar pluripotency of such cells to that of embryonic stem cells (Okita et al., 2007; Wernig et al., 2007). In November 2007, Yamanaka and Thomson laboratories published in Cell and Science, respectively, announcing that they have independently obtained human iPS cells using human skin cells (Takahashi et al., 2007; Yu et al., 2007). In the same year, Jaenisch's research group made progress in treating sickle cell anemia in mice using iPS cell technology. This was the first attempt in the scientific community to use iPS cell technology for therapy research (Hanna et al., 2007). By introducing few simple transcription factors into differentiated somatic cells to reprogram these cells and restore their pluripotency is a revolutionary breakthrough that can provide an in vitro approach to obtaining pluripotent stem cells from patient's own somatic cells. Thus, this approach not only avoids immune rejection, but also avoids the ethical issues. It provides a new way to obtain embryonic stem cells with patient's own genetic background and presents a new prospect for the development of regenerative medicine (Jaenisch and Young, 2008; Yamanaka, 2007).

At present, iPS cell research in the scientific community mainly focuses on two aspects; one is on the improvement of iPS cell technology:

1) the proto-oncogene c-Myc is no longer indispensable in generating iPS cells (Nakagawa et al., 2008; Wernig et al., 2008). In other cell types, Sox2, Klf4, etc. can be omitted or can be substituted by small molecules (Ichida et al., 2009b; Maherali and Hochedlinger, 2009; Shi et al., 2008a; Shi et al., 2008b; Utikal et al., 2009a), and in neural precursor cells, even Oct4 alone can achieve reprogramming (Kim et al., 2009).

2) sources of somatic cells used in iPS cell experiments have expanded from fibroblasts to other cell types (Aoi et al., 2008; Haase et al., 2009; Lowry et al., 2008; Okabe et al., 2009) and from genetically modified somatic cells to somatic cells without genetic modifications (Meissner et al., 2007). Following human and mouse iPS cells, iPS cells derived from rat, monkey and porcine have also been successfully established (Esteban et al., 2009; Liao et al., 2009; Liu et al., 2008; Wu et al., 2009).

3) several methods that can substantially improve the efficiencies of iPS cells have been discovered: inhibition of p53 signaling pathway (Kawamura et al., 2009; Li et al., 2009; Marion et al., 2009; Utikal et al., 2009b); pluripotency-associated microRNA (Judson et al., 2009); TGF signal pathway (Ichida et al., 2009a; Woltjen and Stanford, 2009); Wnt signaling pathway (Marson et al., 2008); SMAD signaling pathway (Chambers et al., 2009); and MAPK signaling pathway (Silva et al., 2008), etc.

4) transfer systems for exogenous genes have been developed from those dependent on viruses to non-viral systems that do not rely on viruses and leave no DNA insertion in the genome (Hotta et al., 2009; Kaji et al., 2009; Okita et al., 2008; Woltjen et al., 2009; Zhou et al., 2009).

Another research focus is on the molecular mechanisms of iPS cell reprogramming. At the moment, researches on the molecular mechanisms of iPS cell reprogramming are still in the exploratory stage. Currently, the four factors mentioned above are thought to re-establish pluripotencies in somatic cell nuclei by the following mechanisms: first, c-Myc expression can initiate DNA replication and in the meantime, loosen the chromatin structures. The loose chromatin structures allow Oct4 to bind to the regulatory regions in the promoters of downstream genes. At the same time, Sox2 and Klf4 can function together with Oct4 to activate and establish transcription factor networks required for pluripotency. These activated transcription factors cooperate with Oct4, Sox2, and Klf4 to activate the process of epigenetic regulation, and eventually establish the epigenomic state of pluripotent cells. In iPS cells, the original repressivehistone modification markers in Oct4 and Nanog promoter regions are replaced with active markers (such as H3K4me and H4Ac), while the methylation state of DNA becomes partially removed. These results indicate that exogenous introduction of Oct4, Sox2, c-Myc and Klf4 can indeed alter the epigenetic state of somatic cells, thereby establishing the pluripotent epigenomic state (Brambrink et al., 2008; Jaenisch and Young, 2008; Maherali et al., 2007; Stadtfeld et al., 2008; Yamanaka, 2007).

The maintenance of pluripotency of iPS cells mainly depends on the activation of endogenous Oct4 and Nanog gene expression. Due to DNA methylation of virus LTR (long terminal repeat) in the reprogramming process, exogenous genes are silenced resulting in low expression (Wernig and et al., 2007). Study using inducible viral expression systems reveal that, after the expression of 4 factors for 10 days, even in the absence of expression of factors from the viruses, the established iPS cells can still divide for a number of generations and maintain stablility in growth characteristics and morphology (Brambrink et al., 2008; Maherali et al., 2007). This indicates that, in the establishment of pluripotency of iPS cell, the functions of ectopic genes carried by the virus are to initiate reprogramming and that the maintenance of pluripotency mainly depends on the expression of endogenous genes.

iPS cells and ES cells share similar characteristics of epigenetic modifications (DNA methylation and histone modification), such as DNA hypomethylation in the promoter regions of pluripotency-associated genes (such as Oct4 and Nanog), tolerance of demethylation of genomic DNA (Wernig et al., 2007). In addition, analysis of the X chromosome in female iPS cell shows that a combination of four transcription factors is sufficient to induce activation of the inactivated X chromosome (X inactivation, Xi), reestablishing the expression of the three non-coding transcription factors that regulate Xi, thereby resetting Xi chromatin modification and removing DNA methylation, which allows random X chromosome inactivation in the subsequent iPS cell differentiation (Maherali et al., 2007).

The presently reported methods for improving efficiency in reprogramming somatic cells to become iPS cells, whether through inhibition of P53 signaling pathway or through use of inhibitors of DNA methyltransferases and histone deacetylases, will undoubtedly trigger nonspecific, unpredictable, large-scale changes at the transcriptome, epigenome, and genome levels. These changes would likely cause genomic instability and the like in the generated iPS cells, thereby hindering the clinical application of these cells.

Therefore, there is still a need for methods that can reprogram somatic cells to become iPS cells with high efficiency. The present invention satisfies these requirements.

### SUMMARY OF THE INVENTION

The present invention provides a type of fusion proteins, which each contain a protein, or a fragment thereof, encoded by a gene associated with cell totipotency and a transcription regulation domain, or a fragment thereof that retains the transcription regulation activity.

In accordance with one embodiment of the invention, a gene associated cell totipotency may be selected from OCT4, NANOG, SOX2, Tcl1, Tcf3, Rex1, Sal4, lefty1, Dppa2, Dppa4, Dppa5, Nr5a1, Nr5a2, Dax1, Esrrb, Utf1, Tbx3, Grb2, Tel1, Sox15, Gdf3, Ecat1, Ecat8, Fbxo15, eRas or Foxd3.

In accordance with one embodiment of the invention, a gene associated with cell totipotency may be selected from OCT4, NANOG or SOX2.

In accordance with one embodiment of the invention, the protein encoded by a gene associated with cell totipotency may be selected from the amino acid sequence at positions 127-352 of Oct4 or the amino acid sequence at positions 1-286 of Oct4.

In accordance with one embodiment of the invention, a transcription regulation domain may be selected from transcription regulation domains of viral proteins.

In accordance with one embodiment of the invention, a transcription regulation domain may be selected from the transcription regulation domain, or a fragment thereof that retains the transcription activity, of viral protein VP16, EBNA2, or E1A, or may be selected from the transcription regulation domain, or a fragment thereof that retains the transcription activity, of yeast Gal4, Oaf1, Leu3, Rtg3, Pho4, Gln3, Gcn4, Gli3, Pip2, Pdr1, Pdr3, Lac9, or Tea1, or may be selected from the transcription regulation domain, or a fragment thereof that retains the transcription activity, of mammalian p53, NFAT, Sp1 (such as Sp1a), AP-2 (such as Ap-2a), Sox2, NF-κB, MLL/ALL, E2A, CREB, ATF, FOS/JUN, HSF1, KLF2, NF-IL6, ESX, Oct1, Oct2, SMAD, CTF, HOX, Sox2, Sox4 or Nanog, or may be selected from the transcription regulation domain, or a fragment thereof that retains the transcription activity, of plant HSF.

In accordance with one embodiment of the invention, the transcription regulation domain may be selected from the transcription regulation domain, or a fragment thereof that retains the transcription activity, of viral protein VP16, or may be selected from the transcription regulation domain, or a fragment thereof that retains the transcription activity, of yeast Gal4, Oaf1, Leu3, Rtg3, Pho4, Gln3 or Gcn4, or may be selected from the transcriptional regulation domain, or a fragment thereof that retains the transcription activity, of mammalian p53, NFAT, Sp1a, Ap-2a, Sox2, NF-κB or Nanog.

In accordance with one embodiment of the invention, the transcription regulation domain may be selected from: the amino acid sequence at positions 446-490 of VP16, the amino acid sequence at positions 437-448 of VP16, the amino acid sequence at positions 768-881 of yeast Gal4, the amino acid sequence at positions 451-551 of human NFκB, the amino acid sequence at positions 8-32 of mouse p53, the amino acid sequence at positions 139-250 of human Sp1a, the amino acid sequence at positions 31-117 of human Ap-2a, the amino acid sequence at positions 121-319 of mouse Sox2, and the amino acid sequence at positions 244-305 of mouse Nanog.

In accordance with one embodiment of the invention, the fusion protein may contain one or more transcription regulation domains, which may be the same or different.

In accordance with one embodiment of the invention, the fusion protein may be selected from: the amino acid sequences represented by SEQ ID NO: 74-76 and 92-129.

In accordance with one embodiment of the invention, the transcription regulation domain of a viral protein may be the transcription regulation domain of VP16 protein encoded by herpes simplex virus.

In accordance with one embodiment of the invention, the transcription regulation domain may be selected from the transcription regulation domains of yeasts.

In accordance with one embodiment of the invention, the transcription regulation domain may be selected from the transcription regulation domains, or fragments thereof, of yeast Gal4, Oaf1, Leu3, Rtg3, Pho4, Gln3, Gcn4, Gli3, Pip2, Pdr1, Pdr3, Lac9, or Tea1.

In accordance with one embodiment of the invention, the transcription regulation domain may be selected from the transcription regulation domains of yeast Gal4, Oaf1, Leu3, Rtg3, Pho4, Gln3 or Gcn4.

In accordance with one embodiment of the invention, the transcription regulation domains are selected from the transcription regulation domains, or fragment thereof, of mammalian p53, NFAT, Sp1(such as Sp1a), AP-2 (such as Ap-2a), Sox2, NF-κB, MLL/ALL, E2A, CREB, ATF, FOS/JUN, HSF1, KLF2, NF-IL6, ESX, Oct1, Oct2, SMAD, CTF, HOX, AP-2, Sox2, Sox4 or Nanog.

In accordance with one embodiment of the invention, the transcription regulation domain may be selected from the transcription regulation domains of mammalian p53, NFAT, Sp1a, Ap-2a, Sox2 or NF-κB.

In accordance with one embodiment of the invention, the transcriptional regulation domain, which may be linked to an N or C terminus of a protein encoded by a gene associated with cell totipotency, is capable of reprogramming somatic cells to become iPS cells with high efficiency.

In accordance with one embodiment of the invention, a protein encoded by a gene associated with cell totipotency may be linked to a transcription regulation domain by a glycine linker.

In accordance with one embodiment of the invention, the linker may be selected from: G(SGGGG)₂SGGGLGSTEF, RSTSGLGGGS(GGGGS)₂G, QLTSGLGGGS(GGGGS)₂G, QLTSGLGGGS(GGGGS)₂G, G(SGGGG)₂SGGGLGSTEF, and RSTSGLGGGS(GGGGS)₂G.

In accordance with one embodiment of the invention, the tandem sequence is a tandem sequence of two or three of the amino acid sequences at positions 446-490 of VP16 or the amino acid sequences at positions 437-448 of VP16.

The present application provides a type of nucleotide sequences, each of which encodes a fusion protein of the present application.

In accordance with one embodiment of the invention, the fusion protein is as described above.

In accordance with one embodiment of the invention, the nucleotide sequence may be selected from SEQ ID NO: 71, SEQ ID NO: 72, SEQ ID NO: 73 and SEQ ID NO: 77-91.

The present application provides a type of expression vectors, each of which expresses a fusion protein of the present application.

In accordance with one embodiment of the invention, the expression vector may express any one of the amino acid sequences of SEQ ID NO: 74-76 and 92-129.

In accordance with one embodiment of the invention, the expression vector may contain a nucleotide sequence of the present application.

In accordance with one embodiment of the invention, the expression vector may contain any one of the nucleotide sequences of SEQ ID NO: 71, SEQ ID NO: 72, SEQ ID NO: 73 or SEQ ID NO: 77-91.

In accordance with one embodiment of the invention, the expression vector may be a lentiviral vector.

The present application provides a type of compositions, each of which may contain a fusion protein, a nucleotide sequence and/or an expression vector, and a carrier or excipient of the present application.

In accordance with one embodiment of the invention, the composition may contains at least one fusion protein selected from the group consisting of: a fusion protein formed by fusing OCT4 protein with a transcription regulation domain of VP16 of herpes simplex virus, a fusion protein formed by fusing NANOG with a transcription regulation domain of VP16 of herpes simplex virus, a fusion protein formed by fusing SOX2 with a transcription regulation domain of VP16 of herpes simplex virus, and a fusion protein formed by fusing Oct4 with a transcription regulation domain of yeast Gal4 or human NFκB or mouse p53 or human Sp1a or human Ap-2a or mouse Sox2 or mouse Nanog.

In accordance with one embodiment of the invention, the composition may further comprises Klf4 protein.

contain any one of the nucleotide sequences of SEQ ID NO: 71, 72, 73 or SEQ ID NO: 77-91 and/or any one of the amino acid sequences of SEQ ID NO: 74-76 and 92-129.

The present application provides a method for reprogramming somatic cells to become induced pluripotent stem cells or other cell lineages with different functions, the method comprising:

(1) treating somatic cells with a fusion protein, a nucleotide sequence, an expression vector, or a composition of the present invention,

(2) screening the treated somatic cells for cells with physicochemical characteristics of pluripotent stem cells or cells of other lineages to obtain induced pluripotent stem cells or cells of other linages with different functions.

The cells of other lineages include cardiac muscle cells, blood cells (such as platelets and immune cells), nerve cells, etc.

In accordance with one embodiment of the invention, a method may comprise introducing a fusion protein, a nucleotide sequence, an expression vector and/or a composition described above into somatic cells through viruses, plasmid transfections, protein transductions, and/or mRNA transfections.

In accordance with one embodiment of the invention, somatic cells may be reprogrammed to become induced pluripotent stem cells using episomal plasmids.

The present invention provides a type of reagent kits. A reagent kit may containa fusion protein, a nucleotide sequence, an expression vector, or a composition of the instant application.

The present invention provides a type of cells. A cell may contain a fusion protein, an expression vector, and/or a nucleotide sequence of the instant application.

In accordance with one embodiment of the invention, the cells are not human embryonic stem cells.

In accordance with one embodiment of the invention, the cells may be induced pluripotent stem cells.

In accordance with one embodiment of the invention, the cells may contain any one of the sequences represented by SEQ ID NO: 71-129.

### BRIEF DESCRIPTION OF THE DRAWINGS

FIG. 1 shows that synthetic factors improve the reprogramming efficiency. a. Diagram used for constructing synthetic factors for reprogramming MEF cells. GL: Glycine linker. Oct4, Sox2, and Nanog, respectively, is fused with VP16 activation domain. b. Comparison of the number of AP and Oct4-GFP positive iPS cell clones at 15 days after retrovirus infection. Different combinations of retroviral vectors as indicated in the figure. O: Oct4; S: Sox2; K: Klf4. The standard deviation is calculated based on the results of three independent experiments. c. Morphology of iPS cell clones generated by the induction of XYKZ factor. Clones at 14 days after virus infection are shown in the figure. Scale indicates 200 μM.

FIG. 2 shows the identification of mouse iPS cells generated by the induction of synthetic factors. a. The iPS cells generated by the induction of Klf4 and synthetic factor X, Y, Z possess typical morphology of ES cells. As shown in the figure, iPS cells uniformly express Oct4-GFP, and show AP staining positive. The scale is 200 μm. b. Positive immunofluorescence staining of pluripotency marker genes SSEA-1 and Nanog in iPS cells. The scale is 200 μm. c. RT-PCR detection of the expression of key ES marker genes. GAPDH serves as a sample control. d. Quantitative RT-PCR detection of transcription levels of Oct4, Nanog, Sox2, and Klf4 of virus origin in 6 strains of iPS cell lines, showing that the expression of exogenous genes of virus origin have been silenced. Actin serves as a sample control. The corresponding gene expression levels in MEF cells at 4 days after virus infection serve as baseline. e. Comparison of methylation levels of bisulfite sequencing in Oct3/4 and Nanog gene promoter region in iPS cells, ES and MEF cells. Hollow circle represent unmethylated CpG, solid circle represents methylated CpG. The iPS cells and ES cells are the same, in that de-methylation occurs in Oct4 and Nanog gene promoter regions.

FIG. 3 shows mouse iPS cells exhibiting pluripotency generated by the induction of artificial factors. a. Comparison of gene expression profiles of iPS cells, ES and MEF cells, confirming that the iPS cells and ES cells are relatively close. b. Chimeric mice produced by mouse iPS cells and offsprings of germline transmission. IPS cell line was microinjected into blastocysts of ICR mice to produce chimeric mice, and through germline transmission to produce offsprings. The contribution of iPS cells leads to the generation of chimeric mice and their offsprings having the wild-type color and colored eyes. c. E13.5 day embryos generated by tetraploid embryos compensation methods. IPS cells were microinjected into tetraploid blastocysts of ICR chimeric blastocysts to produce chimeric blastocysts, which were then transplanted into a surrogate mother for continued growth. d. The ability of germline contribution of XYKZ iPS cells. IPS cells were microinjected into blastocysts of ICR mice. GFP signals in the reproductive ridges of E13.5 day chimeric embryos indicate that iPS cells have been incorporated into germ line.

FIG. 4 shows that synthetic factors increase the production efficiency of human iPS cells. a. Numbers of iPS cell clones produced by infecting 5×10⁵ human foreskin fibroblasts with lentivirus particles containing synthetic factors of three factors (XYK) or four factors (XYKZ), much higher than the numbers of clones produced by the corresponding natural factors. B. Typical in situ diagram of human iPS cells produced by the induction of a combination of synthetic factors XYKZ. After cell clones are established, clone morphology appeared normal and detected positive for alkaline phosphatase AP. P4 refers to cells passed to the fourth generation. The scale is 200 μm. c. Immunofluorescence testing positive for pluripotency marker genes OCT4, NANOG, SOX2, SSEA4, TRA-1-60 and TRA-1-81 of human iPS cells. All scales are 200 μm. d. RT-PCR detection of pluripotency marker gene expression in iPS cells. e. Using in vitro differentiation methods to confirm the pluripotency of human iPS cells. Immunohistochemical staining by antibodies that label three germ layers shows positive on the differentiated iPS cells. The scale is 100 μm. DAPI (blue) stains for nuclei. f. Using in vivo identification method to verify the produced human iPS cells having pluripotency. Subcutaneous injection of human iPS cells into nude mice producing teratomas, which contain different tissue types that belong to the three germ layers. g. Human iPS cells possess normal karyotype.

FIG. 5 shows the expression of synthetic transcription factor in MEF cells. The annotations on the left indicate the antibodies used in Western experiments.

FIG. 6 shows comparison of reactivation kinetics of pluripotent genes in MEF cells infected by virus. After RNA samples were isolated from each sample shown in the figure, detection was carried out using semi-quantitative RT-PCR. Virus-infected, GFP-expressing MEF cells serve as a negative control. Use of synthetic factors causes early activation of endogenous Oct4 gene, the expression can be clearly detected on the sixth day.

FIG. 7 shows comparison of demethylation kinetics of Oct4 promoter region in MEF cells infected by virus. After DNA samples isolated from each sample shown in the figure, detection was carried out using COBRA and bisulfite sequencing methods. It shows that the use of synthetic factors can more easily cause demethylation of the endogenous Oct4 gene promoter in MEF cells.

FIG. 8 shows the kinetics of reprogramming in MEF cells and demethylation of DNA. a. FACS shows the kinetics of reactivation of SSEA-1 and Oct4-GFP in MEF cells at 6, 9, and 12 day after infection of virus containing three combinations of reprogramming factors (OSKN, OSKN+p53sh, and XYKZ). When a combination of synthetic factors XYKZ is used, the numbers of SSEA-1 and Oct4-GFP single-positive and double-positive cells increase at various time points. b. DNA methylation analysis on Oct4 promoter region in cell subpopulations obtained from MEF infected by three groups of reprogramming factors through flow cytometry cell sorting. DNA samples are obtained and prepared from each time point in each cell subpopulation and are analyzed by using COBRA. White arrow head-indicated bands reflect the levels of demethylation in Oct4 region. The greatest demethylation occurred in SSEA-1/GFP double-positive cells infected with XYKZ at 12^{th} day.

FIG. 9 shows the results that compare the kinetics in number of iPS cell clones produced. a. FACS results show that more GFP positive cells (24.7%) appeared in MEF cells 9 days after XYKZ virus infection. Signals detected in PE channel serve as an auto-fluorescence control. b. 21 days after XYKZ infection, more GFP positive clones were generated. Figures show clones grown in the media. c. OG2-MEF infected by DsRed and XYKZ or OSKN retroviruses 2-day post-infection were processed through FACS to sort DsRed positive MEF cells to 96-well plate (one cell per well), for each combination, sorting to 10 96-well plates. At 10-day and 20 day post-sorting, GFP+/DsRed− and GFP+/DsRed+ iPS clones were counted. GFP+ reflects the activation of endogenous Oct4, DsRed− indicates silence of retroviral vectors.

FIG. 10 shows exogenous expression of synthetic factors did not affect the expression levels of p53, p21 and p16. Figures show the results of Western analysis of MEF cells infected by retrovirus carrying XYKZ factors.

FIG. 11 shows pluripotent iPS cells can be produced by a synthetic factor Oct4-VP16. a. Kinetic curves of reprogrammed MEF cells induced by Oct4-VP16 and Oct4-3×VP16. After MEF cells were infected by viruses carrying Oct4 and Oct4 fusion protein genes, the GFP positive iPS clones were counted everyday from 9-day to 17-day post-infection. Three VP16 linked in series enhance the ability of synthetic factors to reprogram. b. Normal morphology of iPS cell lines established by and iPS clones produced by Oct4-VP16 induction. The scale is 250 μm. c. Immunofluorescence experiments show that iPS cells produced by Oct4-VP16 express the pluripotency marker genes Oct4, Nanog and SSEA-l. The scale is 100 μm. d. Detection of totipotency gene expression in Oct4-VP16 iPS cells by quantitative PCR. Expression levels in MEF cells are set to 1. The detected expression levels of 5 totipotency genes approximate that of ES cell line R1. e. Genomic PCR confirmed the existence of Oct4 transgene introduced by retrovirus in only iPS cell lines established by Oct4-VP16. f. iPS cells produced by using a single factor Oct4-VP16 can generate chimeric mice (black arrow) and are capable of germline transmission (white arrow).

FIG. 12 shows the use of an episomal plasmid carrying synthetic factors capable of producing iPS cells without DNA insertion from mouse somatic cells with high efficiency. a. An episomsal plasmid map used for iPS induction. OCT4-VP16, KLF4, SOX2-VP16 and NANOG-VP16 coding sequencesare sequentially linked through 2A elements and then cloned into pCEP4 vector. b. Normal morphology of iPS clone and cell lines generated by pCEP4-XKYZ induction. P5 refers to cells passed to the fifth generation. The scale is 200 μm. c. PCR analysis shows that the genomes of iPS cells generated by the plasmid do not contain plasmid insertion. Using genomic DNA of iPS cells generated by plasmid induction and MEF cells as templates, and a mixture of pCEP4-XKYZ plasmid DNA and MEF cell genomic DNA as a positive control, using primers specific to the transgene and the vector backbone for PCR amplification to detect the insertion of plasmid DNA. d. Chimeric mouse obtained by microinjection of iPS cell clone No. 2 into ICR mouse blastocysts. The chimeric mouse exhibits agouti (color) and colored eyes, indicating incorporation of iPS cells. e. iPS cells have the ability to incorporate into germ line. iPS cells were microinjected into ICR mouse blastocysts. At E13.5 days, GFP positive signals in the reproductive ridge of chimeric embryos indicate that iPS cells are capable of entering the reproductive system.

FIG. 13 shows the identification of mouse iPS cells generated by episomal plasmid induction, a. Using Southern hybridization analysis to show that the iPS cell genomes do not have plasmid DNA. Digest 15 μg of genomic DNA with restriction enzyme EcoRV and, after membrane transfer, hybridize it with the probe. The diluted plasmid DNA serves as positive control. b. Immunostaining shows the expression of Oct4, Nanog and SSEA-1 in iPS cells. The scale is 100 μm. c. Quantitative PCR analysis showed the normal expression of totipotency gene in iPS cells. d, e. Comparison of gene expression profiles of iPS cells generated by plasmid and MEF cells and ES cells shows close relationship with ES cells. f. Normal karyotype of iPS cells.

FIG. 14 shows Genbank numbers and sequences of VP16, yeast Gal4, human NFκB, mouse p53, human Sp1a, human Ap-2a, mouse Sox2 and mouse Nanog. Underlines indicate the amino acid sequences used for fusion.

FIG. 15 shows the fusion amino acid sequences of Tcl1, Tcf3, Rex1, Sal4, lefty1, Dppa2, Dppa4, Dppa5, Nr5a1, Nr5a2, Dax1, Esrrb, Utf1, Tbx3, Grb2, Tel1, Sox15, Gdf3, Ecat1, Ecat8, Fbxo15, eRas, and Foxd3 with VP16 AD (446-490).

### DETAILED DESCRIPTION

The first aspect of the application provides a fusion protein. The fusion protein contains proteins encoded by totipotency-related genes or their fragments and fragments of transcription regulatory domains or fragments having transcription activity.

In this disclosure, “cell totipotency related” means genes associated with regulation, control, production, or restoration of cell totipotency. Genes related to cell totipotency include OCT4, NANOG, SOX2, Tel1, Tcf3, Rex1, Sal4, lefty1, Dppa2, Dppa4, Dppa5, Nr5a1, Nr5a2, Dax1, Esrrb, Utf1, Tbx3, Grb2, Tel1, Sox15, Gdf3, Ecat1, Ecat8, Fbxo15, eRas and Foxd3 etc.

In some embodiments, the fusion proteins of the present invention may also contain active fragments of cell totipotency related genes. Examples of active fragments include but are not limited to the amino acid sequences of Oct4 127-352 and Oct4 1-286.

In this disclosure, “transcription regulatory domain” means the regulation (e.g., activation or repression) of transcription by an amino acid sequence of 30-100 amino acid residues, rich in acidic amino acids, rich in glutamine, rich in proline and other different types, usually acidic structural domains, include fragments of transcription regulatory domains and fragments of domains having transcription regulation function of VP16, EBNA2, E1A, Gal4, Oaf1, Leu3, Rtg3, Pho4, Gln3, Gcn4, Gli3, Pip2, Pdr1, Pdr3, Lac9, Tea1, p53, NFAT, Sp1 (e.g., Sp1a), AP-2 (e.g., Ap-2a), Sox2, NF-κB, MLL/ALL, E2A, CREB, ATF, FOS/JUN, HSF1, KLF2, NF-1L6, ESX, Oct1, Oct2, SMAD, CTF, HOX, Sox2, Sox4 or Nanog, etc.

Other transcription regulatory domains can be used in the present invention and can be selected from fragments of plant HSF transcription regulatory domains or fragments having transcription regulation function.

Examples of transcription regulatory domains or fragments having transcription regulation function include but are not limited to the amino acid sequence of VP16 446-490, the amino acid sequence of VP16 437-448, the amino acid sequence of yeast Gal4 768-881, the amino acid sequence of human NFκB 451-551, the amino acid sequence of mouse p53 8-32, the amino acid sequence of human Sp1a 139-250, the amino acid sequence of Ap-2a 31-117, the amino acid sequence of mouse Sox2 121-319, and the amino acid sequence of mouse Nanog 244-305.

The fusion proteins of the present invention may contain one or more of the same or different transcriptional regulatory domains. These same or different transcriptional regulatory domains can be directly connected with each other in series. They can also be connected through linker sequences.

Examples of serially linked transcription regulatory domains include but are not limited to 3 fragments of tandemly-linked VP16 446-490 shown in SEQ ID NO:81, and 2 fragments of serially-linked SEQ ID NO:82 shown in VP16 437-448.

The instant application can use the transcription regulatory domains of viral proteins, such as VP16, EBNA2, and E1A. In one embodiment, the viral proteins may be selected from herpes simplex virus encoded VP16 protein. In one specific embodiment, the transcription regulatory domain used is fragments of the transcription activator domain and fragments having transcription regulatory function of herpes simplex virus encoded VP16 protein.

In addition, fragments of transcription regulatory domains and fragments having transcription regulatory function of transcription factors represented by yeast Gal4, Oaf1, Leu3, Rtg3, Pho4, Gln3, Gcn4, Gli3, Pip2, Pdr1, Pdr3, Lac9, Tea1 and by mammalianp53, NFAT, Sp1 (e.g., Sp1a), AP-2 (e.g., Ap-2a), Sox2, NF-κB, MLL/ALL, E2A, CREB, ATF, FOS/JUN, HSF1, KLF2, NF-1L6, ESX, Oct1, Oct2, SMAD, CTF, HOX, Sox2, Sox4 or Nanog, etc. can be used in the instant application.

In the instant application, mammals include human, mouse, etc.

Therefore, fusion proteins of the instant application may be proteins of OCT4, SOX2 and/or NANOG proteins fused with the transcription regulatory domains of herpes simplex virus encoded VP16 protein.

In fusion proteins of the instant application, proteins encoded by the genes related to cell totipotency or their fragments can be directly linked to fragments of transcription regulatory domains or fragments having transcription activity, or may contain linker sequences used for linking proteins encoded by the genes related to cell totipotency and the transcription regulatory domains, for example, used to link OCT4, SOX2 and/or NANOG proteins and the transcription regulatory domains of herpes simplex virus encoded VP16 protein. The linker sequences are preferably glycine linker sequences. The number of glycine in linker sequences is not specifically limited, usually 2-40, such as 2-30, 2-25, 2-20, 2-15, 2-10, 2-8 or 3-30, 3-25, 3-20, 3-15, 3-10, or more than 4, or less than 30, 25, 20, 15, 12 or 10.

Examples of fusion proteins of the instant application include the amino acid sequence of any one of fusion proteins, such as SEQ ID NO: 74-76 and 92-129.

The second aspect of the instant application provides a nucleotide sequence, which encodes the fusion protein of the instant application.

Specifically, nucleotide sequences of the instant application contain nucleotide sequences of cell totipotency related genes or their fragments, and coding sequences of transcription regulatory domains or their fragments.

Cell totipotency/pluripotency related genes include OCT4, NANOG, SOX2, Tel1, Tcf3, Rex1, Sal4, lefty1, Dppa2, Dppa4, Dppa5, Nr5a1, Nr5a2, Dax1, Esrrb, Utf1, Tbx3, Grb2, Tel1, Sox15, Gdf3, Ecat1, Ecat8, Fbxo15, eRas and Foxd3, etc.

Polynucleotide sequences of the present invention may include the full-length sequences of these cell pluripotency related genes or fragments thereof.

Transcription regulatory domains include fragments of transcription regulatory domains and fragments of the domains having transcription regulation function of VP16, EBNA2, E1A, Gal4, Oaf1, Leu3, Rtg3, Pho4, Gln3, Gcn4, Gli3, Pip2, Pdr1, Pdr3, Lac9, Tea1, p53, NFAT, Sp1 (e.g., Sp1a), AP-2 (e.g., Ap-2a), Sox2, NF-κB, MLL/ALL, E2A, CREB, ATF, FOS/JUN, HSF1, KLF2, NF-IL6, ESX, Oct1, Oct2, SMAD, CTF, HOX, Sox2, Sox4 or Nanog, etc.

In specific embodiments, the nucleotide sequences contain coding sequences for OCT4, SOX2 and/or NANOG proteins and coding sequences for transcription regulatory domains of herpes simplex virus VP16 protein, Gal4, p53, NFAT, Sp1a, Ap-2a, Sox2 or NF-κB. In other embodiments, coding sequence for poly-glycine linkers may be added between the coding sequences for OCT4, SOX2 and/or NANOG protein and the coding sequences for transcription regulatory domain of herpes simplex virus VP16 protein.

In a preferred embodiment, the nucleotide sequence of the present invention is selected from any one nucleotide sequence: coding for the amino acid sequences shown in SEQ ID NO:74-76 and 92-129.

In other preferred embodiments, the nucleotide sequence is selected from SEQ ID NO: 71, SEQ ID NO: 72, SEQ ID NO: 73 and SEQ ID NO:77-91.

The third aspect of the instant application provides a method for reprogramming somatic cells to become induced pluripotent stem cells or other cell lineages having different functions, the method includes:

(1) treating somatic cell with the fusion proteins, nucleotide sequences, expression vectors or compositions of the present invention.

(2) after culturing, screening for cells having physicochemical properties of pluripotent stem cells or cells of other lineages to obtain induced pluripotent stem cells or cells of other lineages with different functions.

A specific method can be divided into the following steps:

1, Transferring a fusion protein or a nucleotide sequence of the present invention into a somatic cell by viral infection, plasmid transfection, protein transduction, or mRNA transfection.

2, After culturing for a period of time, selecting the produced iPS clones to establish stable iPS cell lines.

3, Determining gene expression and developmental pluripotency of the established iPS cell lines.

The fourth aspect of the instant application provides a kind of iPS cells and methods of the instant application for obtaining same. iPS cells obtained by using methods of the instant application through the presence of DNA insertion of technical means have unique insertion sequences in the genomes. These unique insertion sequences of coding sequences for fusion proteins of the present invention include but not limited to coding sequences of fusion proteins of OCT4, SOX2 and/or NANOG protein with transcription regulatory domain (especially herpes simplex virus encoded VP16 protein, Gal4, p53, NFAT, Sp1a, Ap-2a, Sox2 or NF-κB transcription regulatory domains).

The fifth aspect of the instant application provides a test kit, including the proteins, nucleotide sequences and/or expression vectors of the instant application. The kit may also contain other reagents suitable for delivering the proteins and/or the nucleotide sequences. The kit may also contains a description, used to direct the technical person using the kit to treat somatic cells, reprogramming somatic cells to become induced pluripotent stem cells or through combinations of different factors to induce somatic cells to become other types of cells.

The sixth aspect of the instant application provides use of transcription regulatory domain for the manufacture of reagents for reprogramming somatic cells to become induced pluripotent stem (iPS) cells. The reagents include fusion proteins, such as fusion proteins of the instant application. The transcription regulatory domain may be selected from fragments of transcription regulatory domains and fragments, within the domains, having transcription regulation function of VP16, EBNA2, E1A, Gal4, Oaf1, Leu3, Rtg3, Pho4, Gln3, Gcn4, Gli3, Pip2, Pdr1, Pdr3, Lac9, Tea1, p53, NFAT, Sp1a (Sp1a), AP-2 (such as Ap-2a), Sox2, NF-κB, MLL/ALL, E2A, CREB, ATF, FOS/JUN, HSF1, KLF2, NF-IL6, ESX, Oct1, Oct2, SMAD, CTF, HOX, Sox2, Sox4 or Nanog, etc. More preferably transcription regulatory domains can be selected from the transcription structural domain of herpes simplex virus encoded VP16 protein, and optionally may be selected from yeast Gal4, Oaf1, Leu3, Rtg3, Pho4, Gln3 and Gcn4 and, in mammals, p53, NFAT, Sp1a, Ap-2a, Sox2, and NF-κB as representative transcription regulatory domains of transcription factors.

In the instant application, OCT4, NANOG, SOX2 proteins, and transcription regulatory domains may be any known OCT4, NANOG, SOX2 proteins, and transcription regulatory domains (especially the transcription regulatory domains of herpes simplex virus encoded VP16 protein), including their derivatives or analogues that retain the required properties, activity, and/or structures. Particularly preferred derivatives or analogs include essentially conservative substitutions, which occur at the amino acids with related side chains. Specifically, amino acids can be generally divided into four categories: (1) acid—aspartic acid and glutamic acid; (2) basic—lysine, arginine, and histidine; (3) nonpolar—alanine, valine, leucine, isoleucine, phenylalanine, proline, methionine, and tryptophan; and (4) polar uncharged—glycine, asparagine, glutamine, serine, threonine, cysteine, and tyrosine. Sometimes, phenylalanine, tryptophan and tyrosine are classified as aromatic amino acids. For example, it is reasonable to predict: individually substitute isoleucine or valine with leucine, glutamic acid with aspartic acid, serine with threonine, or substitute similar conservative amino acids with structurally related amino acids. This kind of substitutions would not cause important impact on biological activity. For example, peptides of interest may include up to about 5-10 conservative or non-conservative amino acid substitutions, or even up to about 15-25 conservative or non-conservative amino acid substitutions, or any integer between 2 and 25, as long as the functions required in the molecules remain intact. One skilled in the art can combine the Hopp/Woods and Kyte-Doolittle curves known in the art and easily determine the regions that can tolerate changes in the molecules of interest.

Nucleotide sequences of the instant application encoding OCT4, NANOG, SOX2 proteins, and sequences of transcription regulatory domains (especially the transcription regulatory domains of herpes simplex virus encoded VP16 protein) include the sequences encoding their analogs or derivatives, as long as these coding sequences, after being introduced into the cells, express OCT4, NANOG, SOX2 proteins and transcription regulatory domains and achieve their original functions and/or activity.

One skilled in the art would understand that, through Blast, one can easily search homologous amino acid sequences and coding nucleotide sequences of OCT4, NANOG, SOX2 proteins, and transcription regulatory domains used in the instant application, including but not limited to, the amino acid sequences and coding nucleotide sequences listed in the following tables. These sequences can also be used for the instant application, as long as these sequences, after being introduced into the cells, express OCT4, NANOG, SOX2 proteins and transcription regulatory domains and achieve their original functions and/or activity.

Homologous sequences of transcription regulatory domains

| Transcription | | |
|---|---|---|
| regulatory | | |
| domains | Blast query | Genbank Accession No. |
| Nanog | naaplhnfgedflqpyvqlqqnfsasdlevnleatresha | NP_082292.1 |
| | hfstpqalelflnysvtppgei | NP_001074414.1 |
| | | NP_001094251.1 |
| | | XP_001498858.1 |
| | | XP_002344677.1 |
| | | NP_079141.2 |
| | | XP_002344676.1 |
| | | XP_002822903.1 |
| | | NP_001065295.1 |
| | | XP_002822902.1 |
| | | XP_002712808.1 |
| | | XP_001088535.1 |
| | | XP_002752349.1 |
| | | XP_002752348.1 |
| | | XP_002763050.1 |
| | | XP_002806052.1 |
| | | XP_001119249.2 |
| | | XP_543828.2 |
| | | XP_002723815.1 |
| | | XP_001112736.1 |
| | | XP_001112791.1 |
| | | NP_001166913.1 |
| | | NP_001020515.1 |
| | | YP_003694332.1 |
| | | ZP_05843703.1 |
| p53 | qsdislelplsqetfsglwkllppe | NP_001120705.1 |
| | | NP_035770.2 |
| | | NP_112251.2 |
| | | NP_001075873.1 |
| | | XP_002747997.1 |
| | | XP_002747996.1 |
| | | XP_002747995.1 |
| | | XP_002747994.1 |
| | | NP_001166211.1 |
| | | NP_001040616.1 |
| | | XP_002827022.1 |
| | | XP_002827021.1 |
| | | XP_002827020.1 |
| | | XP_002827019.1 |
| | | NP_001119586.1 |
| | | XP_001172077.1 |
| | | XP_511957.2 |
| | | NP_001119585.1 |
| | | NP_000537.3 |
| | | NP_998989.2 |
| | | NP_999310.1 |
| | | NP_001189334.1 |
| | | XP_002924483.1 |
| | | NP_001003210.1 |
| | | NP_001009294.1 |
| | | NP_776626.1 |
| | | NP_001009403.1 |
| | | NP_001001903.1 |
| | | NP_001081567.1 |
| | | NP_001118164.1 |
| | | XP_001485464.1 |
| | | XP_001196748.1 |
| Gal4 | anfnqsgniadsslsftftnssngpnlittqtnsqalsqpia | NP_015076.1 |
| | ssnvhdnfmnneitaskiddgnnskplspgwtdqtayn | XP_002499285.1 |
| | afgittgmfntttmddvynylfddedtppnpkke | XP_002555997.1 |
| | | XP_453627.1 |
| | | NP_349190.1 |
| | | ZP_07377189.1 |
| | | YP_003729861.1 |
| Spla | nrtvsggqyvvaaapnlqnqqvltglpgvmpniqyqvi | NP_612482.2 |
| | pqfqtvdgqqlqfaatgaqvqqdgsgqiqiipganqqii | NP_003100.1 |
| | tnrgsggniiaampnllqqavplqglannvlsgqt | XP_509098.2 |
| | | XP_001104948.1 |
| | | XP_002823363.1 |
| | | XP_001104877.1 |
| | | NP_036787.2 |
| | | XP_002752575.1 |
| | | XP_002711174.1 |
| | | NP_038700.2 |
| | | XP_002923324.1 |
| | | NP_001071495.1 |
| | | XP_001926920.2 |
| | | XP_543633.2 |
| | | XP_858079.1 |
| | | NP_989935.1 |
| | | NP_989139.1 |
| | | NP_001084888.1 |
| | | XP_002667905.1 |
| | | XP_001495308.2 |
| | | XP_001514802.1 |
| | | NP_989934.1 |
| | | XP_852053.1 |
| | | XP_002922454.1 |
| | | XP_002749367.1 |
| | | XP_001928818.2 |
| | | XP_002685342.1 |
| | | XP_613036.3 |
| | | XP_515917.2 |
| | | NP_001083425.1 |
| | | XP_001926617.2 |
| | | XP_002712234.1 |
| | | XP_002934651.1 |
| | | XP_001088331.2 |
| | | XP_002933342.1 |
| | | NP_001153261.1 |
| | | NP_003102.1 |
| | | NP_001166183.1 |
| | | XP_001376384.1 |
| | | NP_001017371.3 |
| | | XP_002729217.1 |
| | | XP_002726235.1 |
| | | NP_001018052.1 |
| | | NP_001091895.1 |
| | | XP_001370863.1 |
| | | NP_001159857.1 |
| | | XP_001101092.2 |
| | | XP_418708.2 |
| | | NP_036893.1 |
| | | NP_033265.3 |
| | | XP_539462.1 |
| | | XP_862942.1 |
| | | XP_002751551.1 |
| | | XP_001497703.1 |
| | | NP_003103.2 |
| | | XP_002707857.1 |
| | | XP_002930521.1 |
| | | XP_003130245.1 |
| | | XP_002818208.1 |
| | | NP_997827.1 |
| | | NP_001082967.1 |
| | | NP_001122096.1 |
| | | XP_002713861.1 |
| | | XP_002191146.1 |
| | | XP_423405.2 |
| | | XP_001372651.1 |
| | | XP_002834288.1 |
| | | NP_003101.3 |
| | | XP_001173433.1 |
| | | XP_511930.2 |
| | | XP_001083602.1 |
| | | XP_001083816.1 |
| | | XP_001917645.1 |
| | | XP_002748587.1 |
| | | XP_002916889.1 |
| | | NP_001015654.1 |
| | | NP_001093452.1 |
| | | XP_691096.4 |
| | | NP_956418.1 |
| | | NP_001074433.1 |
| | | NP_084496.2 |
| | | ZP_06756169.1 |
| AP-2 ( [symbol] Ap-2a) | lgtvgqspytsapplshtpnadfqppyfpppyqpiypqs | NP_001027451.1 |
| | qdpyshvndpyslnplhaqpqpqhpgwpgqrqsqes | XP_001166719.1 |
| | gllhthrglphq | NP_003211.1 |
| | | NP_001035890.1 |
| | | XP_857355.1 |
| | | XP_848968.1 |
| | | XP_001491137.1 |
| | | NP_035677.2 |
| | | XP_002915950.1 |
| | | XP_002714212.1 |
| | | XP_001924601.1 |
| | | NP_001073697.1 |
| | | XP_001924657.1 |
| | | NP_001100815.1 |
| | | NP_001116420.1 |
| | | XP_002714211.1 |
| | | XP_002915948.1 |
| | | XP_001924634.1 |
| | | XP_857563.1 |
| | | XP_857524.1 |
| | | XP_001367436.1 |
| | | XP_001367479.1 |
| | | NP_001009745.1 |
| | | XP_001166555.1 |
| | | XP_001166516.1 |
| | | XP_857440.1 |
| | | XP_857399.1 |
| | | XP_002915949.1 |
| | | XP_001514228.1 |
| | | XP_001514207.1 |
| | | NP_001089041.1 |
| | | NP_001032335.1 |
| | | NP_001158795.1 |
| | | NP_789829.1 |
| | | XP_001089006.2 |
| | | XP_001149872.1 |
| | | XP_002714542.1 |
| | | XP_001363947.1 |
| | | XP_001363860.1 |
| | | NP_001020476.1 |
| | | XP_518532.1 |
| | | XP_001149948.1 |
| | | XP_001502945.1 |
| | | NP_001002977.1 |
| | | XP_001106196.2 |
| | | NP_990226.1 |
| | | XP_002714541.1 |
| | | XP_001502935.1 |
| | | NP_001069715.1 |
| | | NP_033360.2 |
| | | XP_001149667.1 |
| | | NP_001100366.2 |
| | | XP_001509705.1 |
| | | XP_001509737.1 |
| | | NP_001087701.1 |
| | | NP_001039109.1 |
| | | XP_001509668.1 |
| | | XP_001509631.1 |
| | | NP_001019836.1 |
| | | NP_001017847.1 |
| | | XP_002720918.1 |
| | | NP_001011093.1 |
| | | XP_002830497.1 |
| | | XP_001093748.2 |
| | | NP_003213.1 |
| | | XP_002747749.1 |
| | | NP_001116673.1 |
| | | XP_001156107.1 |
| | | NP_001123400.1 |
| | | XP_867402.1 |
| | | XP_543065.2 |
| | | XP_867413.1 |
| | | NP_001083186.1 |
| | | NP_001068977.1 |
| | | NP_958823.2 |
| | | NP_033361.2 |
| | | XP_002199252.1 |
| | | XP_417497.2 |
| | | NP_001153168.1 |
| | | XP_417778.2 |
| | | XP_001368947.1 |
| | | XP_001368984.1 |
| | | XP_782460.2 |
| | | NP_001161503.1 |
| | | NP_001008576.1 |
| | | XP_002921260.1 |
| | | NP_957115.1 |
| Sox2 | lmkkdkytlpggllapggnsmasgvgvgaglgagvnq | NP_035573.3 |
| | rmdsyahmngwsngsysmmqeqlgypqhpglnah | XP_002921878.1 |
| | gaaqmqpmhrydvsalqynsmtssqtymngsptys | NP_003097.1 |
| | msysqqgtpgmalgsmgsvvkseasssppvvtssshs | NP_001098933.1 |
| | rapcqagdlrdmismylpgaevpepaapsrlhmaqhy | NP_001136412.1 |
| | qsgpvpgtaingtlplshm | NP_001166918.1 |
| | | XP_545216.2 |
| | | XP_002807611.1 |
| | | XP_002716497.1 |
| | | NP_990519.1 |
| | | NP_001116669.1 |
| | | XP_516895.2 |
| | | NP_998869.1 |
| | | NP_001081691.1 |
| | | NP_001137271.1 |
| | | XP_001506984.1 |
| | | NP_001135190.1 |
| | | NP_998283.1 |
| | | XP_001368820.1 |
| | | XP_002199662.1 |
| | | NP_989526.1 |
| | | NP_001098234.1 |
| | | NP_001007502.1 |
| | | NP_001084148.1 |
| | | NP_001001811.2 |
| | | NP_001166875.1 |
| | | XP_001367325.1 |
| | | XP_866541.1 |
| | | XP_866526.1 |
| | | NP_001158337.1 |
| | | XP_002832243.1 |
| | | XP_002720573.1 |
| | | NP_033263.2 |
| | | XP_852934.1 |
| | | XP_549298.2 |
| | | NP_001180681.1 |
| | | NP_005625.2 |
| | | NP_001032751.1 |
| | | XP_002832242.1 |
| | | XP_001915911.1 |
| | | NP_001002483.1 |
| | | NP_989664.1 |
| | | XP_001364938.1 |
| | | NP_001089143.1 |
| | | XP_002742590.1 |
| | | XP_002724136.1 |
| | | XP_002593021.1 |
| | | XP_875555.1 |
| | | XP_002824485.1 |
| | | XP_849239.1 |
| | | NP_033259.2 |
| | | NP_005977.2 |
| | | XP_001520781.1 |
| | | NP_001074465.1 |
| | | XP_002191049.1 |
| | | XP_001143467.1 |
| | | NP_571777.1 |
| | | NP_999639.1 |
| | | NP_001158377.1 |
| | | NP_570983.1 |
| | | XP_001516120.1 |
| | | XP_002430102.1 |
| | | YP_001323187.1 |
| | | ZP_01462099.1 |
| | | YP_003950003.1 |
| | | XP_002408658.1 |
| | | XP_001377782.1 |
| | | XP_002415541.1 |
| NF-κB | gallgnstdpavftdlasvdnsefqqllnqgipvaphttep | NP_068810.3 |
| | mlmeypeaitrlvtgaqrppdpapaplgapglpngllsg | XP_001170004.1 |
| | dedfssiadmdfsallsqiss | XP_001170057.1 |
| | | XP_002821553.1 |
| | | NP_001138610.1 |
| | | XP_001113258.2 |
| | | XP_002916737.1 |
| | | XP_002799530.1 |
| | | XP_002807426.1 |
| | | XP_540850.2 |
| | | NP_001073711.1 |
| | | XP_001249719.2 |
| | | NP_001107753.1 |
| | | NP_033071.1 |
| | | NP_954888.1 |
| | | XP_001490867.2 |
| | | XP_001379658.1 |
| | | XP_001170021.1 |
| | | NP_001001211.1 |
| | | NP_001081048.1 |
| | | XP_003027956.1 |
| | | NP_990460.1 |
| | | ZP_07274894.1 |
| VP16 | dalddfdldmlgdgdspgpgftphdsapygaldmadf | NP_044650.1 |
| | efeqmftdalgideygg | NP_044518.1 |
| | | YP_164491.1 |
| | | NP_851908.1 |
| | | YP_443895.1 |
| | | YP_003933792.1 |
| | | XP_970024.1 |

Homologous sequences of transcription factors

| Transcription | | |
|---|---|---|
| factors | Blast query | Genbank Accession No. |
| Nanog | msvglpgphslpsseeasnsgnassmpavfhpenyscl | NP_082292.1 |
| | qgsatemlcteaasprpssedlplqgspdsstspkqklssp | NP_001074414.1 |
| | eadkgpeeeenkvlarkqkmrtvfsqaqicalkdrfqkq | NP_001094251.1 |
| | kylslqqmqelssilnlsykqvktwfqnqrmkekrwqk | XP_001498858.1 |
| | nqwlktsngliqkgsapveypsihcsypqgylvnasgsl | NP_079141.2 |
| | smwgsqtwtnptwssqtwtnptwnnqrwtnptwssq | XP_002344676.1 |
| | awtaqswngqpwnaaplhnfgedflqpyvqlqqnfsa | NP_001065295.1 |
| | sdlevnleatreshahfstpqalelflnysvtppgei | XP_002822902.1 |
| | | XP_001112791.1 |
| | | NP_001020515.1 |
| | | NP_001166913.1 |
| | | XP_002752348.1 |
| | | NP_001123443.1 |
| | | XP_002763050.1 |
| | | XP_002344677.1 |
| | | XP_001088535.1 |
| | | XP_002712808.1 |
| | | XP_543828.2 |
| | | XP_002822903.1 |
| | | XP_001112736.1 |
| | | XP_002752349.1 |
| | | XP_002723815.1 |
| | | XP_001139456.1 |
| | | XP_001119249.2 |
| | | XP_001367968.1 |
| | | XP_001516194.1 |
| | | XP_002190732.1 |
| | | NP_001139614.1 |
| | | XP_002190766.1 |
| | | XP_001900019.1 |
| | | NP_001071957.1 |
| OCT4 | maghlasdfafspppgggdgsaglepgwvdprtwlsfq | NP_038661.2 |
| | gppggpgigpgsevlgispcppayefcggmaycgpqv | NP_001009178.1 |
| | glglvpqvgvetlqpegqagarvesnsegtssepcadrp | XP_001490158.1 |
| | navklekveptpeesqdmkalqkeleqfakllkqkritlg | NP_001108427.1 |
| | ytqadvgltlgvlfgkvfsqtticrfealqlslknmcklrpll | XP_002746363.1 |
| | ekwveeadnnenlqeicksetlvqarkrkrtsienrvrws | NP_002692.2 |
| | letmflkcpkpslqqithianqlglekdvvrvwfcnrrqk | NP_001106531.1 |
| | gkrssieysqreeyeatgtpfpggavsfplppgphfgtpg | XP_002809144.1 |
| | ygsphfttlysvpfpegeafpsvpvtalgspmhsn | NP_001166912.1 |
| | | XP_002752527.1 |
| | | NP_001093427.1 |
| | | XP_001135162.1 |
| | | NP_777005.1 |
| | | NP_001153014.1 |
| | | XP_528230.1 |
| | | XP_538830.1 |
| | | XP_001148833.1 |
| | | NP_976034.4 |
| | | XP_001134983.1 |
| | | XP_001375906.1 |
| | | NP_083591.1 |
| | | NP_001075220.1 |
| | | XP_001139199.1 |
| | | XP_002713962.1 |
| | | NP_694948.1 |
| | | XP_002942017.1 |
| | | NP_001081342.1 |
| | | NP_001103648.1 |
| | | XP_002190361.1 |
| | | NP_001098339.1 |
| | | NP_001079832.1 |
| | | NP_001123406.1 |
| | | NP_571187.1 |
| | | NP_001087461.1 |
| | | NP_032926.2 |
| | | NP_620192.1 |
| | | NP_006227.1 |
| | | XP_002941201.1 |
| | | NP_571225.1 |
| | | XP_539052.2 |
| | | XP_001083202.1 |
| | | XP_001926276.1 |
| | | NP_032925.1 |
| | | NP_005595.2 |
| | | NP_001073817.1 |
| | | XP_001371773.1 |
| | | XP_002922031.1 |
| | | NP_742082.1 |
| | | XP_001787898.1 |
| | | NP_001090220.1 |
| | | XP_002691240.1 |
| | | NP_958855.1 |
| | | NP_571177.2 |
| | | XP_525843.2 |
| | | XP_001501398.2 |
| | | XP_589021.2 |
| | | XP_002720190.1 |
| | | XP_001501084.1 |
| | | XP_001925801.1 |
| | | NP_001181188.1 |
| | | XP_001370024.1 |
| | | XP_002930080.1 |
| | | NP_032927.1 |
| | | XP_002831894.1 |
| | | NP_571236.1 |
| | | NP_001158054.1 |
| | | XP_549108.1 |
| | | NP_571235.1 |
| | | XP_002913559.1 |
| | | XP_002746893.1 |
| | | NP_001096655.1 |
| | | NP_001016504.1 |
| | | NP_001086347.1 |
| | | XP_002710046.1 |
| | | NP_001026755.1 |
| | | NP_620193.1 |
| | | XP_002934918.1 |
| | | NP_002690.3 |
| | | NP_001094393.1 |
| | | NP_000298.2 |
| | | XP_002720688.1 |
| | | NP_001098921.1 |
| | | NP_035271.1 |
| | | XP_003127845.1 |
| | | XP_782909.2 |
| | | NP_001081583.1 |
| | | XP_001364853.1 |
| | | XP_003127848.1 |
| | | NP_001090728.1 |
| | | NP_571364.1 |
| | | XP_001916635.1 |
| | | XP_850049.1 |
| | | XP_002411292.1 |
| | | XP_002035616.1 |
| | | XP_002007467.1 |
| | | XP_001518292.1 |
| | | NP_001139385.1 |
| | | XP_002414009.1 |
| | | XP_002093959.1 |
| | | XP_001971533.1 |
| SOX2 | mynmmetelkppgpqqasgggggggnataaatggnq | NP_035573.3 |
| | knspdrvkrpmnafmvwsrgqrrkmaqenpkmhns | NP_001136412.1 |
| | eiskrlgaewkllsetekrpfideakrlralhmkehpdyk | NP_990519.1 |
| | yrprrktktlmkkdkytlpggllapggnsmasgvgvga | XP_002921878.1 |
| | glgagvnqrmdsyahmngwsngsysmmqeqlgyp | NP_003097.1 |
| | qhpglnahgaaqmqpmhrydvsalqynsmtssqtym | NP_001098933.1 |
| | ngsptysmsysqqgtpgmalgsmgsvvkseasssppv | NP_001166918.1 |
| | vtssshsrapcqagdlrdmismylpgaevpepaapsrlh | NP_001116669.1 |
| | maqhyqsgpvpgtaingtlplshm | XP_516895.2 |
| | | XP_002716497.1 |
| | | XP_545216.2 |
| | | XP_002807611.1 |
| | | NP_998869.1 |
| | | NP_001081691.1 |
| | | NP_001137271.1 |
| | | NP_001135190.1 |
| | | NP_998283.1 |
| | | XP_001506984.1 |
| | | XP_001368820.1 |
| | | NP_001098234.1 |
| | | NP_001001811.2 |
| | | XP_002199662.1 |
| | | NP_989526.1 |
| | | NP_001084148.1 |
| | | NP_001007502.1 |
| | | NP_001166875.1 |
| | | XP_001367325.1 |
| | | NP_001158337.1 |
| | | XP_866541.1 |
| | | XP_866526.1 |
| | | XP_002832243.1 |
| | | NP_033263.2 |
| | | XP_002720573.1 |
| | | NP_001032751.1 |
| | | XP_852934.1 |
| | | XP_549298.2 |
| | | NP_001002483.1 |
| | | NP_005625.2 |
| | | NP_001180681.1 |
| | | XP_002832242.1 |
| | | XP_001915911.1 |
| | | XP_002593021.1 |
| | | NP_001089143.1 |
| | | NP_989664.1 |
| | | XP_001364938.1 |
| | | XP_002724136.1 |
| | | XP_875555.1 |
| | | XP_002742590.1 |
| | | NP_001158377.1 |
| | | XP_002824485.1 |
| | | NP_571777.1 |
| | | NP_033259.2 |
| | | XP_849239.1 |
| | | NP_005977.2 |
| | | NP_001074465.1 |
| | | NP_999639.1 |
| | | NP_570983.1 |
| | | XP_001632997.1 |
| | | XP_001952682.1 |
| | | XP_002430102.1 |
| | | XP_002415541.1 |
| | | NP_001100320.1 |
| | | XP_002196106.1 |
| | | XP_002814130.1 |
| | | NP_004180.1 |
| | | NP_990092.1 |
| | | XP_542802.2 |
| | | XP_002918684.1 |
| | | XP_002645043.1 |
| | | NP_001158461.1 |
| | | XP_001365271.1 |
| | | NP_001158346.1 |
| | | NP_001009888.1 |
| | | XP_001078360.2 |
| | | XP_003118339.1 |
| | | NP_001158344.1 |
| | | NP_001032769.1 |
| | | NP_999638.1 |
| | | NP_808421.1 |
| | | XP_319093.4 |
| | | NP_009015.1 |
| | | XP_001084162.1 |
| | | XP_001366447.1 |
| | | NP_001093703.1 |
| | | XP_001366503.1 |
| | | NP_001165685.1 |
| | | XP_002939326.1 |
| | | NP_741836.1 |
| | | XP_974496.2 |
| | | XP_002593017.1 |
| | | NP_001165684.1 |
| | | XP_391958.3 |
| | | XP_001897041.1 |
| | | XP_001097044.2 |
| | | XP_002424557.1 |
| | | XP_003140388.1 |
| | | NP_001122329.1 |
| | | NP_001122330.1 |
| | | XP_001511549.1 |
| | | XP_002094767.1 |

Protein or nucleotide sequences of the instant application can be delivered by using various methods. For example, plasmids having nucleotide sequences of the instant application may be introduced into cells for transient expression using transfection reagents (Fugene6, Roche; Lipofectamine, Invitrogen, etc). One also can incubate cells using the protein solutions of the instant application. One can also use conventional culture medium to incubate the obtained cells.

Any somatic cells including any mammalian somatic cells can be used in practicing the methods of the instant application. Preferred mammals are human, mouse, etc.; preferred somatic cells include: skin fibroblasts, blood cells, oral epithelial cells, etc.

After treating somatic cells with protein or nucleotide sequences of the instant application, to determine whether somatic cells have been induced to become iPS cells, known methods, for example, Takahashi, K. & Yamanaka, S. Induction of pluripotent stem cells from mouse embryonic and adult fibroblast cultures by defined factors. Cell 126, 663-676, (2006); Okita, K., Ichisaka, T. & Yamanaka, S. Generation of germline-competent induced pluripotent stem cells. Nature 448, 313-317, (2007), can be used.

The present invention provides a composition. The composition contains the fusion proteins, nucleotide sequences, and/or expression vectors, and vehicles or excipients of the instant application. Vehicles or excipients that can be used in the instant application include vehicles or excipients commonly used in the field. For example, the vehicles or excipients may be compatible with the fusion proteins and may be suitable culture medium components for culturing somatic cells or iPS cells, or may be compatible with the nucleotide sequences and may be suitable components of transforming agents used, for example, for transforming cells. The amounts of the ingredients can be determined, usually based on actual amount needed, by one skilled in the art using conventional techniques.

The present invention provides a reagent kit. The kit may contain the fusion proteins, nucleotide sequences, expression vectors and/or compositions of the instant application. The kit may also contain manual that directs technical personnel using the kit to prepare iPS cells from somatic cells. Kit may also include, for example, reagents used for preparing fusion proteins and for incubating somatic cells using the prepared products, the reagents may be suitable for culturing somatic cells or iPS cells. Or, the kit may include reagents suitable for transfecting nucleotide sequences into cells. Fusion proteins or nucleotide sequences in the kit may be provided in the form of pure substances, prepared with appropriate vehicles or excipients prior to use; or provided in a form of a mixture, such as the composition of the instant application.

The present invention is described by the following embodiments. It should be understood that these embodiments are only illustrative, and not limiting. Unless otherwise indicated, all reagents used are commercially available reagents.

### EXAMPLES

### Materials and Methods

Plasmid construction: The fusion of cDNA encoding mouse and human Oct4, Sox2 and Nanog and encoding VP16 transcription activator domain (VP16 446-490 amino acids, from MLGDG to DEYGG) with or without glycine-rich linker, was cloned into retroviral vector pMXs (Takahashi and Yamanaka, 2006) and lentiviral vector pLV-TRE-EF1a-GFP capable of inducible expression (Wu et al., 2009). To construct episomal plasmids used for iPS induction, DNA encoding OCT4-VP16 (X), KLF4, SOX2-VP16 (Y) and NANOG-VP16 (Z) are connected through 2A elements, and then cloned into epsisomal plasmid vectors pCEP4 (Invitrogen) to produce pCEP4-XKYZ.

Cell culture: Maintain mouse ES cells and iPS cells in DMEM (Invitrogen) on mouse embryonic fibroblast feeder layer (MEF) treated with mitomycin C. DMEM is added with 15% heat-inactivated fetal calf serum (FBS, Invitrogen), 2 mM L-glutamine, 0.1 mM non-essential amino acids, 1 mM sodium pyruvate, 0.1 mM β-mercaptoethanol (Sigma), 1000 units/ml of leukocyte inhibition factor (LIF, Chemicon) and 50 units/50 mg/ml of penicillin and streptomycin. Obtain E13.5 day embryos from crossing male TgOG2 transgenic mice with female wild type C57BL to prepare Oct4-GFP MEF. Grow MEF in DMEM with addition of 10% FBS (Hyclone), 2 mM L-glutamine, 0.1 mM non-essential amino acids, and 100 units/100 mg/ml of penicillin and streptomycin. Use early passages of MEF (up to the fourth passages) to generate iPS cells.

Human ES and iPS cells may be maintained in DMEM with the addition of 20% Knockout serum substitutes (KSR, Invitrogen), 2 mM L-glutamine, 0.1 mM non-essential amino acids, 0.1 mM β-mercaptoethanol, 4 ng/ml basic FGF, (Invitrogen) and 100 units/100 mg/ml of penicillin and streptomycin. Human foreskin fibroblasts obtained from 25 years old normal males were cultured in DMEM containing 10% FBS and 100 units/100 mg/ml of penicillin and streptomycin.

Preparation of retroviruses and induction of mouse iPS cells: perform preparation of retroviruses and infection according to the published scheme (Takahashi, K., Okita, K., Nakagawa, M. & Yamanaka, S., Induction of pluripotent stem cells from fibroblast cultures. Nat Protoc 2, 3081-3089 (2007). Inoculate Plat-E cells (Morita, S., Kojima, T. & Kitamura, T. Plat-E: an efficient and stable system for transient packaging of retroviruses. Gene Ther 7, 1063-1066, 2000) at 7×10⁶ cells per 100-cm Petri dish. Next day, transfect 9 μg pMXs retroviral vector (Addgene) into Plat-E cells using Lipofectamine 2000 reagent (Invitrogen) according to the manufacturer's recommendation. After transfection overnight, replace culture medium. After 48 hours, collect supernatant containing viruses and filter using 0.45 μm PVDF filter paper with 4 μg/ml Polybrene (Sigma) (Millipore). Incubate Oct4-GFP MEF cells (inoculate into 6 well-plates, 5×10⁴ cells per well) with the virus-containing supernatant for 12 hours. 2 days after infection, change culture medium with mouse ES medium. 8 days after infection, re-inoculate Oct4-GFP MEF cells into 6-well plates at 5×10⁴ cells per well on MEF feeder layer treated with mitomycin C treatment. About 7 days after re-inoculation, count the number of GFP positive and alkaline phosphatase-positive clones. Perform alkaline phosphatase staining using NBT/BCIP (Roche) according to the manufacturer's recommendations. Improved culture medium has been used in single factor iPS induction experiments (Chen et al., 2010).

Mouse iPS cells generated by using episomal plasmids: After transfection with 5 μg plasmid pCEP4-XKYZ into 1×10⁶ MEF using electric transfection kit (Amaxa), inoculate the transfected MEF cell into 2 10-cm Petri dishes coated with MEF feeder layer treated with mitomycin C. The second day after transfection, replace the medium with the culture medium of improved formulation (Chen et al., 2010), replace medium every 2 days. About 18 days after transfection, select Oct4-GFP positive iPS clones and perform proliferation and identification.

Human iPS cell induction: infect human foreskin fibroblasts (HFF) at 5×10⁵ inoculated in a 6-cm dish with filtered lentiviral supernatants overnight, and then incubate in HFF medium containing doxycycline (Sigma) up to 1 μg/μL. 2 days after infection, re-inoculate the induced HFF in a ratio 1:3 into MEF feeder layers treated with mitomycin C, and replace the culture medium with human ES culture medium. About 3 weeks after infection, select iPS cell clones, count the number of alkaline phosphatase positive hES-like clones (with round edges, and a diameter greater than 50 μm).

Immunofluorescence analysis. Fix the cells with 4% paraformaldehyde for 30 minutes, then permeate using 0.2% Triton X-100 for 45 minutes, block with 2% BSA (Sigma). Incubate cells in the presence of primary antibody at 4° C. overnight, and then incubate in the presence of secondary antibody at room temperature for 1 hour. Use the following antibodies: SSEA-1 (Santa Cruz), SSEA-4 (R&D), Nanog (Chemicon), Oct4 (Santa Cruz), SOX2 (R&D), TRA-1-60 (Chemicon), TRA-1-81 (Chemicon), FOXA2 (Abeam), SOX17 (Santa Cruz), SMA (AbboMax), BRACHYURY (Abeam), GFAP (Dako), and β-TUBULIN (Covance). Perform alkaline phosphatase staining using Vector Red substrate Kit (Vector Laboratories).

Methods for producing chimeras, transmitting germ line, and compensating tetraploid blastocyst. To produce chimeras, inject iPS cells into ICR E3.5 blastocysts. Next generation generated from chimeras can be used to observe whether iPS cells are transmitted to germline. To produce mice by tetraploid blastocysts compensation methods, electric fuse 2 cell embryos collected from the fallopian tubes of ICR female mice (SLAC Experiment Animal Center) to produce single cell tetraploid embryos, and then culture in KSOM medium (Chemicon). Inject about 10-15 iPS cells into the cavities of tetraploid blastocysts. Maintain the blastocysts in KSOM containing amino acids, until embryo transplantation. Transplant 15-20 injected blastocysts to 2.5 day old uterine horns of ICR female mice with false conception after mating. Obtain the injected tetraploid blastocyst (4N) embryos by dissection at E13.5.

Karyotype analysis of human iPS cell. Treat human iPS cells with 0.1 μg/ml colchicine amide (Invitrogen) at 37° C. for 3 hours, and then treat with trypsin, and suspend in the 0.075M KCl for 20 minutes. Fix cells treated with hypotonic solution in methanol:acetic acid (3:1) at room temperature for 30 minutes. Then, place cells on pre-cleaned slides, stain with DAPI. Count the number of chromosomes in metaphase.

In vitro and in vivo differentiation of human iPS cells. For EB formation, harvest human iPS cells treated with collagenase IV. Transfer cell blocks to a low adhesion dish containing DMEM/F12. DMEM/F12 contains 20% Knockout serum substitutes, 2 mM L-glutamine, 0.1 mM non-essential amino acids and 0.1 mM β-mercaptoethanol. Next day, replace the culture medium. After 8 days of culture in suspension, transfer EB to flat plate of gel pack, and incubate in the same culture medium for 8 days. Test the ability of human iPS cell to differentiate in vivo by using subcutaneous injection, iPS cells that possess totipotency can form teratomas containing three different ectodermal tissues.

Western analysis. Using 2:1 reprogramming factors: infect MEF with pMIG retrovirus (Addgene), collect cell lysates 3 days after infection. Primary antibodies include anti-Oct4 (Santa Cruz), Nanog (Chemicon), Sox2 (Chemicon), Klf4 (SantaCruz), Flag (Sigma), VP16 (Clontech), GFP (Santa Cruz), p53 (Santa Cruz), p21 (Santa Cruz), p16 (Santa Cruz) and), and β-actin (Sigma).

RT-PCR. Isolate total RNA using TRIZOL (Invitrogen) and 1 μg for analyzing cDNA using ReverTra Ace First-Strand cDNA synthesis Kit (Toyobo) according to the manufacturer's recommendations. The PCR primers are shown in Table 1 below. EvaGreen (Stratagene) is used to perform quantitative PCR.

TABLE 1
Primers used in PCR reactions
Mouse iPS cells RT-PCR

| | |
|---|---|
| Endo-Oct4 | Forward: TCTTTCCACCAGGCCCCCGGCTC (SEQ ID NO: 1) |
| | Reverse: TGCGGGCGGACATGGGGAGATCC (SEQ ID NO: 2) |
| Endo-Sox2 | Forward: TAGAGCTAGACTCCGGGCGATGA (SEQ ID NO: 3) |
| | Reverse: TTGCCTTAAACAAGACCACGAAA (SEQ ID NO: 4) |
| Endo-Nanog | Forward: TAGGCTGATTTGGTTGGTGTCTTG (SEQ ID NO: 5) |
| | Reverse: AGTGTGATGGCGAGGGAAGG (SEQ ID NO: 6) |
| Tg-gfp | Forward: AGAAGAACGGCATCAAGG (SEQ ID NO: 7) |
| | Reverse: GCTCAGGTAGTGGTTGTC (SEQ ID NO: 8) |
| Esg1 | Forward: GAAGTCTGGTTCCTTGGCAGGATG (SEQ ID NO: 9) |
| | Reverse: ACTCGATACACTGGCCTAGC (SEQ ID NO: 10) |
| Dax1 | Forward: TGCTGCGGTCCAGGCCATCAAGAG (SEQ ID NO: 11) |
| | Reverse: GGGCACTGTTCAGTTCAGCGGATC (SEQ ID NO: 12) |
| eRas | Forward: ACTGCCCCTCATCAGACTGCTACT (SEQ ID NO: 13) |
| | Reverse: CACTGCCTTGTACTCGGGTAGCTG (SEQ ID NO: 14) |
| Rex1 | Forward: ACGAGTGGCAGTTTCTTCTTGGGA (SEQ ID NO: 15) |
| | Reverse: TATGACTCACTTCCAGGGGGCACT (SEQ ID NO: 16) |
| Zfp296 | Forward: CCATTAGGGGCCATCATCGCTTTC (SEQ ID NO: 17) |
| | Reverse: CACTGCTCACTGGAGGGGGCTTGC (SEQ ID NO: 18) |
| Ecat1 | Forward: TGTGGGGCCCTGAAAGGCGAGCTGAGAT (SEQ ID NO: 19) |
| | Reverse: ATGGGCCGCCATACGACGACGCTCAACT (SEQ ID NO: 20) |
| Thy1 | Forward: AGAAGGTGACCAGCCTGACA (SEQ ID NO: 21) |
| | Reverse: GTTCTGAACCAGCAGGCTTA (SEQ ID NO: 22) |
| Dnmt3a2 | Forward: CTCACACCTGAGCTGTACTGCAGAG (SEQ ID NO: 23) |
| | Reverse: CTCCACCTTCTGAGACTCTCCAGAG (SEQ ID NO: 24) |
| Dnmt3b | Forward: TTCAGTGACCAGTCCTCAGACACGAA (SEQ ID NO: 25) |
| | Reverse; TCAGAAGGCTGGAGACCTCCCTCTT (SEQ ID NO: 26) |
| Dnmt3L | Forward: GTGCGGGTACTGAGCCTTTTTAGA (SEQ ID NO: 27) |
| | Reverse: CGACATTTGTGACATCTTCCACGTA (SEQ ID NO: 28) |
| Ink4a | Forward: GTGTGCATGACGTGCGGG (SEQ ID NO: 29) |
| | Reverse: GCAGTTCGAATCTGCACCGTAG (SEQ ID NO: 30) |
| Arf | Forward: GCTCTGGCTTTCGTGAACATG (SEQ ID NO: 31) |
| | Reverse: TCGAATCTGCACCGTAGTTGAG (SEQ ID NO: 32) |
| Gapdh | Forward: AGTCAAGGCCGAGAATGGGAAG (SEQ ID NO: 33) |
| | Reverse: AAGCAGTTGGTGGTGCAGGATG (SEQ ID NO: 34) |

Quantitative PCR specific for viral transcription factors

| | |
|---|---|
| Viral-X | Forward: TCTCCCATGCATTCAAACTG (SEQ ID NO: 35) |
| | Reverse: CTTTTATTTTATCGTCGACC (SEQ ID NO: 36) |
| Viral-Y | Forward: CTGCCCCTGTCGCACATGTG (SEQ ID NO: 37) |
| | Reverse: CTTTTATTTTATCGTCGACC (SEQ ID NO: 38) |
| Viral-Z | Forward: CATCGCAGCTTGGATACAC (SEQ ID NO: 39) |
| | Reverse: GCATTGATGAGGCGTTCC (SEQ ID NO: 40) |
| Viral-Klf4 | Forward: CCTTACACATGAAGAGGCAC (SEQ ID NO:41) |
| | Reverse: CTTTTATTTTATCGTCGACC (SEQ ID NO: 42) |
| β-actin | Forward: GAAATCGTGCGTGACATCAAAG (SEQ ID NO: 43) |
| | Reverse: TGTAGTTTCATGGATGCCACAG (SEQ ID NO: 44) |

Human iPS cells RT-PCR

| | |
|---|---|
| Endo-OCT4 | Forward: GACAGGGGGAGGGGAGGAGCTAGG (SEQ ID NO:45) |
| | Reverse: CTTCCCTCCAACCAGTTGCCCCAAAC (SEQ ID NO: 46) |
| Endo-SOX2 | Forward: GGGAAATGGGAGGGGTGCAAAAGAGG (SEQ ID NO: 47) |
| | Reverse: TTGCGTGAGTGTGGATGGGATTGGTG (SEQ ID NO: 48) |
| Endo-Nanog | Forward: CAGCCCCGATTCTTCCACCAGTCCC (SEQ ID NO: 49) |
| | Reverse: CGGAAGATTCCCAGTCGGGTTCACC (SEQ ID NO: 50) |
| Rex1 | Forward: CAGATCCTAAACAGCTCGCAGAAT (SEQ ID NO: 51) |
| | Reverse: GCGTACGCAAATTAAAGTCCAGA (SEQ ID NO: 52) |
| DPP45 | Forward: ATATCCCGCCGTGGGTGAAAGTTC (SEQ ID NO: 53) |
| | Reverse: ACTCAGCCATGGACTGGAGCATCC (SEQ ID NO: 54) |
| GDF3 | Forward: CTTATGCTACGTAAAGGAGCTGGG (SEQ ID NO: 55) |
| | Reverse: GTGCCAACCCAGGTCCCGGAAGTT (SEQ ID NO: 56) |
| ECAT-1 | Forward: GGAGCCGCCTGCCCTGGAAAATTC (SEQ ID NO: 57) |
| | Reverse: TTTTTCCTGATATTCTATTCCCAT (SEQ ID NO: 58) |
| ECAT15-1 | Forward: GGAGCCGCCTGCCCTGGAAAATTC (SEQ ID NO: 59) |
| | Reverse: TTTTTCCTGATATTCTATTCCCAT (SEQ ID NO: 60) |
| GAPDH | Forward: TGTTGCCATCAATGACCCCTT (SEQ ID NO: 61) |
| | Reverse: CTCCACGACGTACTCAGCG (SEQ ID NO: 62) |

Bisulfite PCR

| | |
|---|---|
| Oct4-outside | Forward: GAGGATTGGAGGTGTAATGGTTGTT (SEQ ID NO: 63) |
| | Reverse: CTACTAACCCATCACCCCCACCTA (SEQ ID NO: 64) |
| Oct4-inside | Forward: CAAGCTTGGGTTGAAATATTGGGTTTATTT(SEQ ID NO:65) |
| | Reverse: CGGATCCCTAAAACCAAATATCCAACCATA(SEQ ID NO: 66) |
| Nanog-outside | Forward: AAGTATGGATTAATTTATTAAGGTAGTT (SEQ ID NO: 67) |
| | Reverse: AAAAAACCCACACTCATATCAATATA (SEQ ID NO: 68) |
| Nanog-inside | Forward: AAGTATGGATTAATTTATTAAGGTAGTT (SEQ ID NO: 69) |
| | Reverse: CAACCAAATCAACCTATCTAAAAA (SEQ ID NO: 70) |

DNA microarray. Use total RNA labeled with phycoerythrin from Oct4-GFP MEFs, J1 ES cells, and iPS cells (XSKZ#4 clone). According to the manufacturer's recommendation, hybridize samples with Mouse Genome 4302 Array (Affymetrix). Scan and array using Gene array Scanner 3000 (Affymetrix). Use Affymetrix GCOS1.2 software to analyze the data.

Bisulfite genomic sequencing. Treat genomic DNA with bisulfate as described previously (Li, J. Y. et al. Synergistic function of DNA methyltransferases Dnmt3a and Dnmt3b in the methylation of Oct4 and Nanog. Mol Cell Biol 27, 8748-59 (2007)) and perform nested PCR. To perform sequence analysis, clone the PCR products into human T-vector (Takara), and perform sequencing on a single clone.

Flow cytometry. Harvest culture, and obtain single cell suspension by repeated pipetting and transfer through 40 μm cell filter. Incubate cells using Alexa Fluor® 647 anti-mouse SSEA-1 (BioLegend), and select/analyze in FACSAria (BD Biosciences). Use FlowJo software (Tree Star) to analyze the data.

### Experimental Results

1. Effects of synthetic factors on the process of reprogramming MEF to become iPS cells

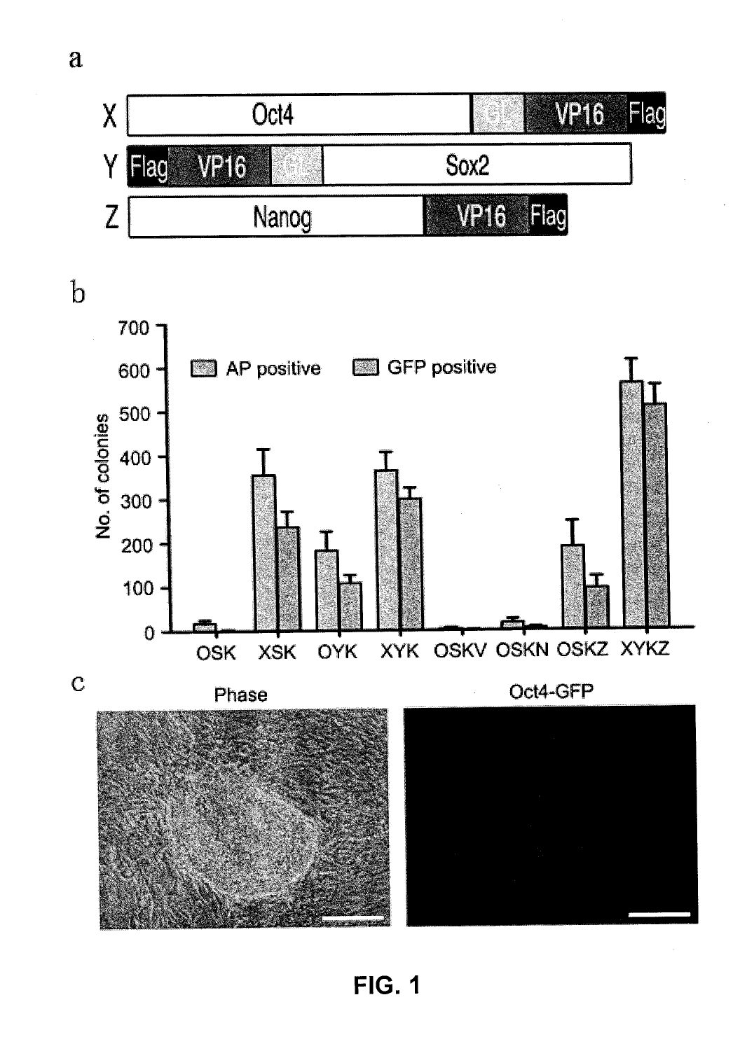

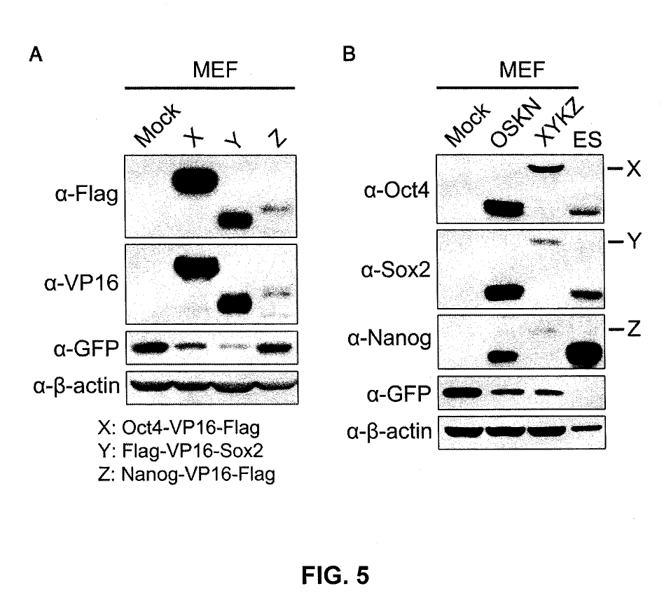

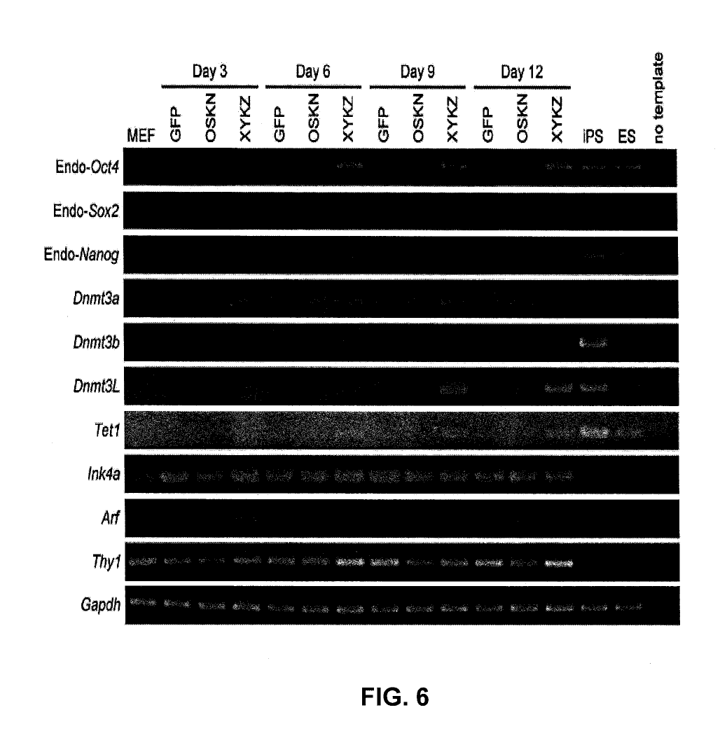

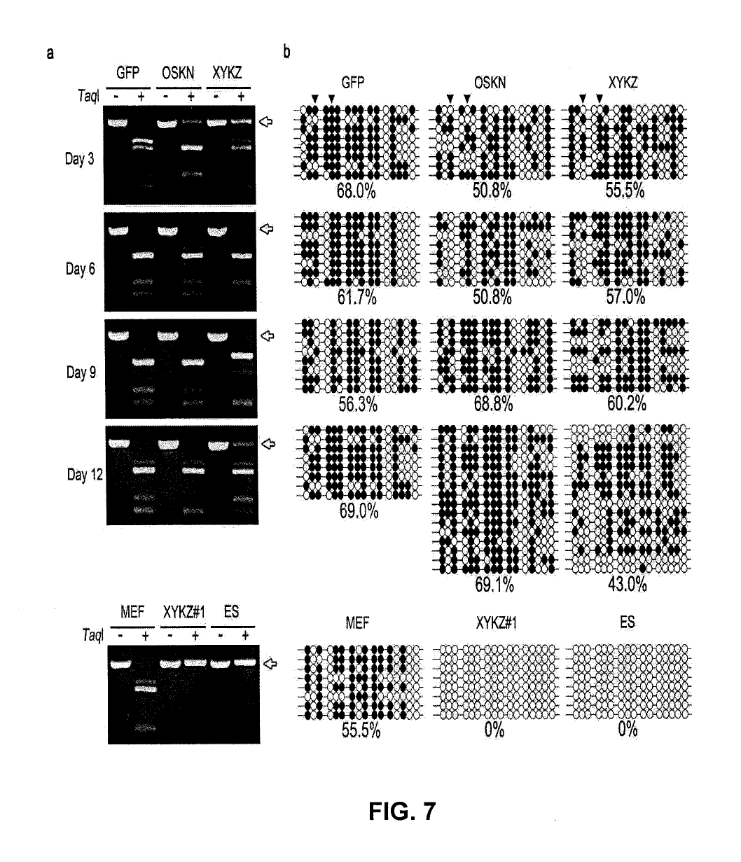

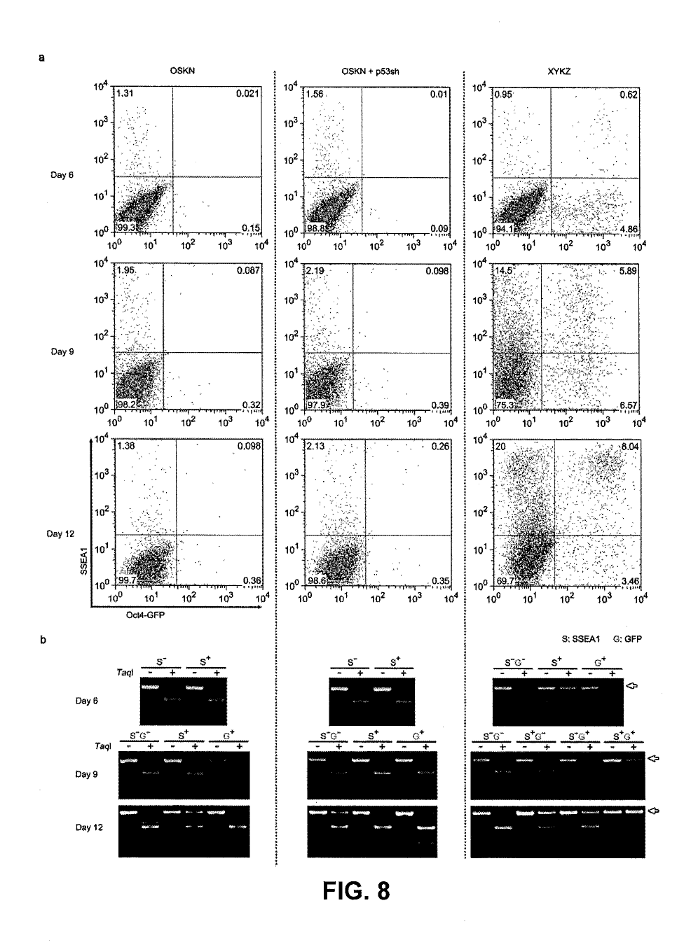

Fusion of the transcription activator domain of herpes simplex virus encoded VP16 protein with Oct4, Sox2 and Nanog, respectively (FIG. 1a). Expression of fusion protein is normal (FIG. 5). After transfection of these factors into MEF cells, reactivation of some stem cell marker genes are detected in the reprogram process. When synthetic factors are used for reprogramming, endogenous genes including Nanog and Oct4 are expressed in the 6th day. Whereas, when natural transcription factors are used, these genes express until the 12^{th} day (FIG. 6). Corresponding to early reactivation of genes is the rapid DNA demethylation in Oct4 promoter region, when synthetic factors are used (FIG. 7). The alkaline phosphatase (AP), SSEA-1 and Oct4-GFP positive cell groups sorted out in the 6th, 9^{th}, and 12^{th} day show higher DNA methylation levels in Oct4 promoter region, thus, linking marker gene reactivation together with DNA demethylation (FIG. 8).

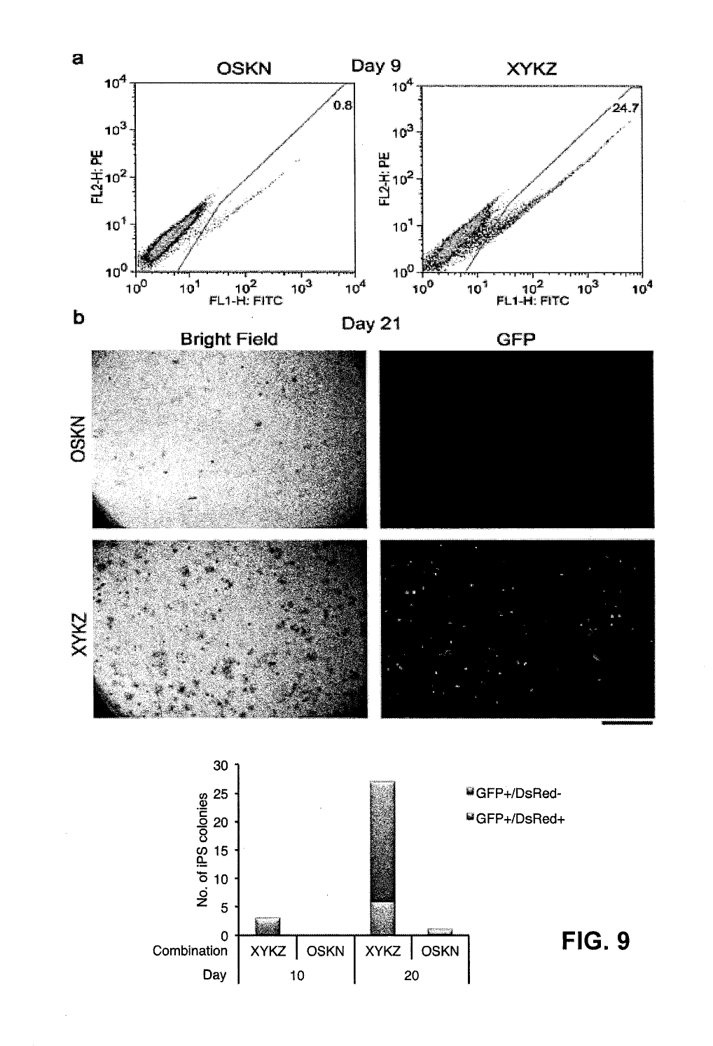

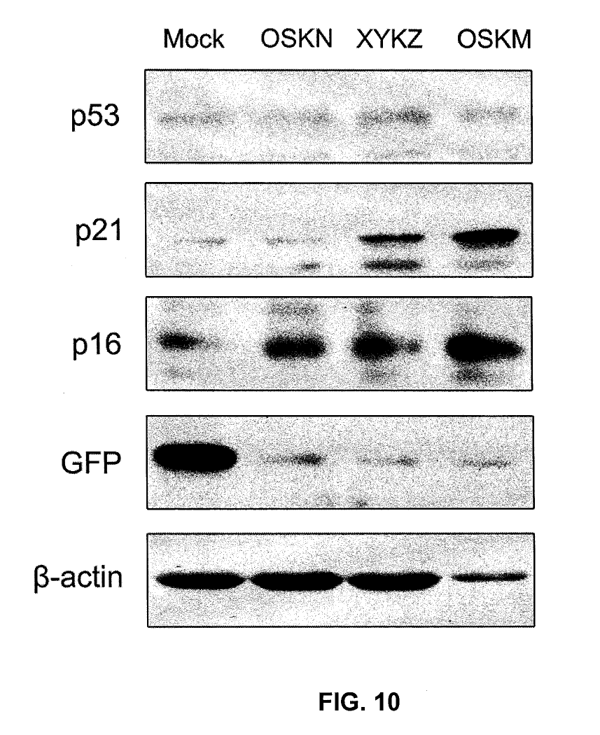

Afterwards, the inventors examined the effects of each fusion protein in the process of reprogramming MEF. The inventors use three-factor system (Oct4, Sox2 and Klf4, abbreviated as OSK) and the four-factor system (OSK plus Nanog), without c-Myc oncogene. When using OSK to reprogram Oct4-GFP transgenic MEF cells, the inventors obtain 3±1 (mean±standard deviation; n=3) GFP positive clones from 5×10⁴ cells (FIG. 1b). In contrast, when using Oct4-VP16 (X) instead of Oct4, 236±35 GFP positive clones are obtained, increasing 78 times. Similarly, using Sox2-VP16 (Y) instead of Sox2, 108±19 GFP positive clones are obtained, increasing 36 times. In four-factor (OSKN) system, when using Nanog-VP16 (Z) instead of Nanog (N), 95±27 clones are obtained, increasing 19 times comparing with that using OSKN to obtain 5±3 clones. Combination of three synthetic factors obtain altogether 511±47 clones, efficiency is increased by more than 100 times (FIG. 1b). The variation in number of AP positive clones corresponds well with the variation in number of GFP positive clones. And, these GFP-positive clones and normal ES cells are not different in cell morphology (FIG. 1c). Most iPS cell clones appear in the 9th day after transfected with XKYZ factors, one week earlier than using natural factors (FIG. 9). These data suggest that synthetic factors can significantly promote reprogramming and increase the number of iPS cells.

2. Identification of iPS cell lines established by synthetic factors

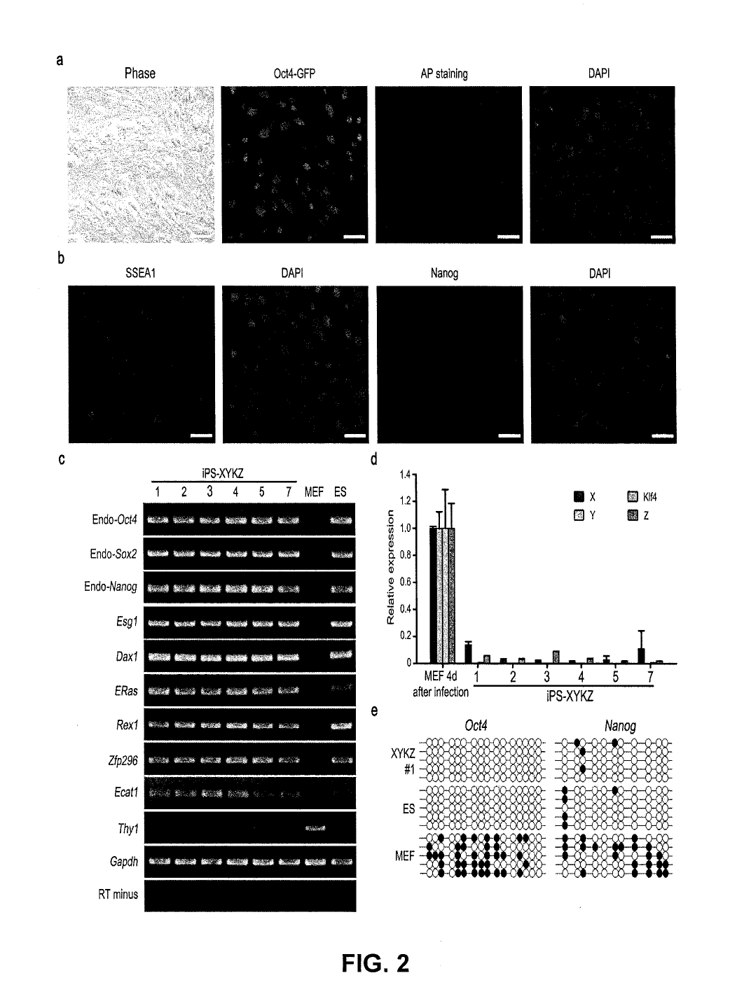

The inventors use different combinations of synthetic factors and produce multiple iPS cell lines (cell clone number 1-5, 7). These cell lines have morphology and proliferation rate similar to that of mouse embryonic stem cells (ES) (FIG. 2a). Their AP activity staining is positive, and they express ES cell surface marker SSEA-1 and nuclear marker Nanog (FIG. 2b). Expression levels of a number of key genes in iPS cells and ES cells remain consistent, including activation of endogenous Oct4, Sox2 and Nanog and decreased expression of Thy1 gene, which is specifically expressed in MEF (FIG. 2c). In iPS cells generated by synthetic factors, the transcription levels of transgenes derived from retroviruses have been silenced to a comparable level similar to that of iPS cells using natural factors (FIG. 2d). Even though the expression of DNA methyltransferase Dnmt3a, Dnmt3b and Dnmt3L is initially up-regulated (FIG. 6), endogenous Oct4 and Nanog are reactivated through DNA demethylation on their promoter regions (FIG. 2e). These results indicate that the epigenetic regulation of iPS cells has reverted to that of a typical ES cell state.

3. iPS cells generated by synthetic factors possess complete developmental totipotency

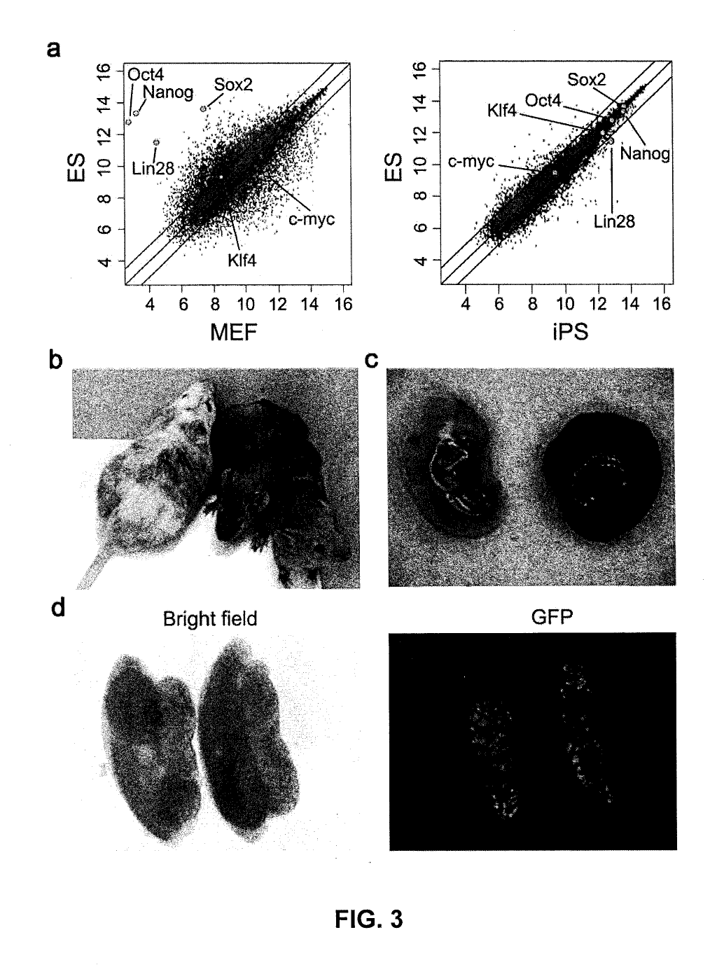

The whole genome gene expression profiles of iPS cells generated by synthetic factors are similar to that of ES cells (FIG. 3a). These cells can contribute to the development of mouse embryos, proving the developmental totipotency of each cell. Injection of IPS cells into diploid blastocysts produce live chimeric mice having highly chimeric coat colors and have offsprings from germline transmission (FIG. 3b). Use of synthetic factors to induce iPS cells can not only through diploid blastocyst injection produce live chimeric mice having highly chimeric coat colors, but also these chimeric mice can, through the germline transmission, produce mice that are generated completely from the iPS cells. Table 2 summarizes the conditions, in which mouse iPS cell line is injected into diploid blastocysts to produce chimeric mice and germline transmission. In addition, the chimeric mice and the mouse offsprings obtained from germline transmission (at least 4 generations) up to now nearly 1 year, free of tumor. In addition, after injection of the iPS cell line (XSKZ #4) into tetraploid blastocysts, live E13.5 mouse embryos are obtained (FIG. 3c). Furthermore, GFP positive cells are found in the genital ridges, indicating that these iPS cells can produce germ cells (FIG. 3d). The above data show that the iPS cells generated by synthetic factor reprogramming have developmental totipotency similar to ES cells.

TABLE 2
Summary of conditions for injection of mouse iPS cell clones into diploid blastocysts

| | | No. of | | No. of | Chimerism | | Germline |
|---|---|---|---|---|---|---|---|
| | Genetic | blastocysts | No. of | surviving | rate | | transmission |
| Clone No. | Background | injected | birth | mouse | >50% | <50% | (number*) |
| XYKZ #1 | OG2 with | 57 | 41 | 29 | 16 | 13 | **No |
| XYKZ #2 | Oct4-GFP | 31 | 24 | 6 | 4 | 2 | No |
| XYKZ #3 | transgene | 47 | 9 | 5 | 3 | 2 | No |
| XYKZ #4 | (C57/CBA) | 41 | 8 | 5 | 2 | 3 | Yes (1) |
| XYKZ #5 | | 44 | 6 | 5 | 2 | 3 | Yes |
| XYKZ #6 | | 53 | 15 | 5 | 1 | 4 | Yes |
| XYKZ #7 | Oct4-GFP | 60 | 20 | 17 | 15 | 2 | Yes (4) |
| XYKZ #8 | Knock-in | 61 | 16 | 9 | 5 | 4 | Yes (2) |
| | (C57/129) | | | | | | |

*“number” in brackets indicates the number of mouse chimeras by germline transmission has been confirmed.
**“No” in brackets indicate that germline transmission was not observed at the time of filing the instant application.

4. Synthetic factors can also promote the generation of human induced pluripotent stem cells

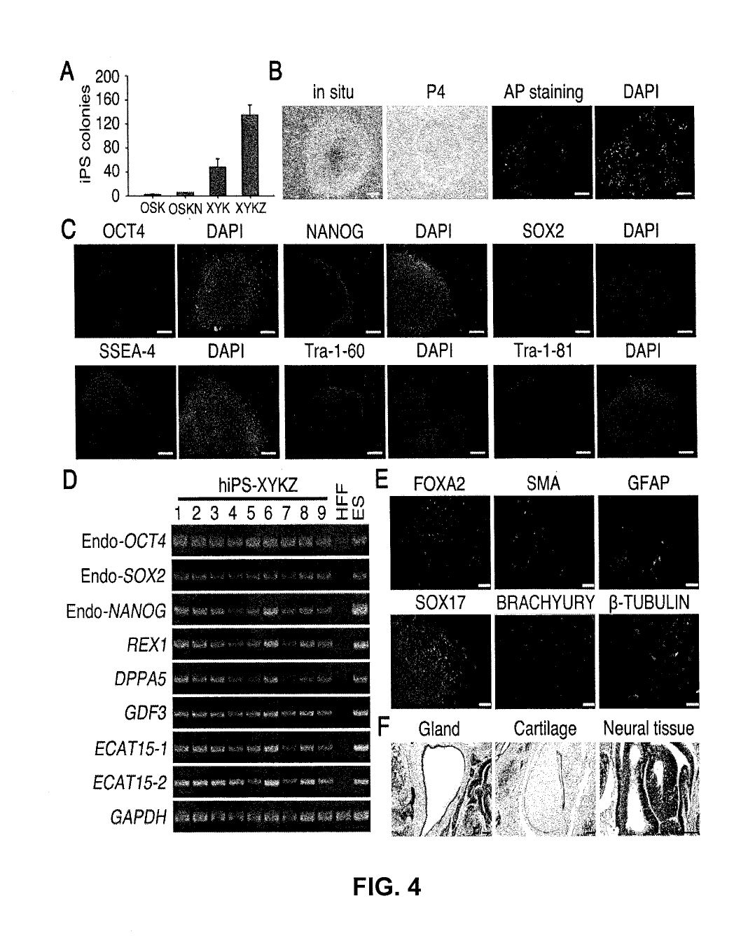

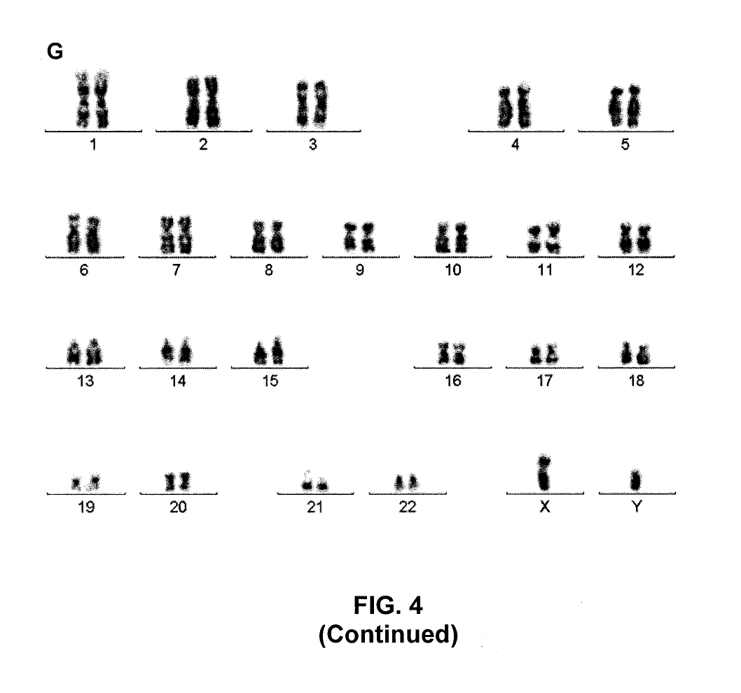

Next, the inventors examine whether synthetic transcription factors can increase the efficiency of generating human induced pluripotent stem cells. Transferring these synthetic factors carried by inducible lentiviral expression vectors to human foreskin fibroblasts to induce iPS cells. Whether in three- or four-factor experimental system, use of synthetic factors can produce significantly more iPS cell clones than natural factors (FIG. 4a). These iPS cells exhibit normal morphology of human embryonic stem cell and are AP positive (FIG. 4b). The following confirms the expression of other markers of human iPS cells Immunofluorescence staining shows that these cells uniformly express ES cell markers OCT4, NANOG, SOX2, SSEA4, TRA-1-60 and TRA-1-81 (FIG. 4c). Analysis of gene expression shows that the expression levels of ES cell markers often seen in iPS cells are equivalent to that of ES cells (FIG. 4d). These iPS cells have normal karyotype (FIG. 4g) and grow in in vitro differentiation medium and in the injected immunodeficient mice to generate three germ layer cell types (FIG. 4e, f). The above results show that synthetic factors not only can improve the production efficiency of mouse iPS cells, but also can improve the efficiency of generating iPS cells from human somatic cells.

5. iPS cells can be induced from somatic cells by using one synthetic factor

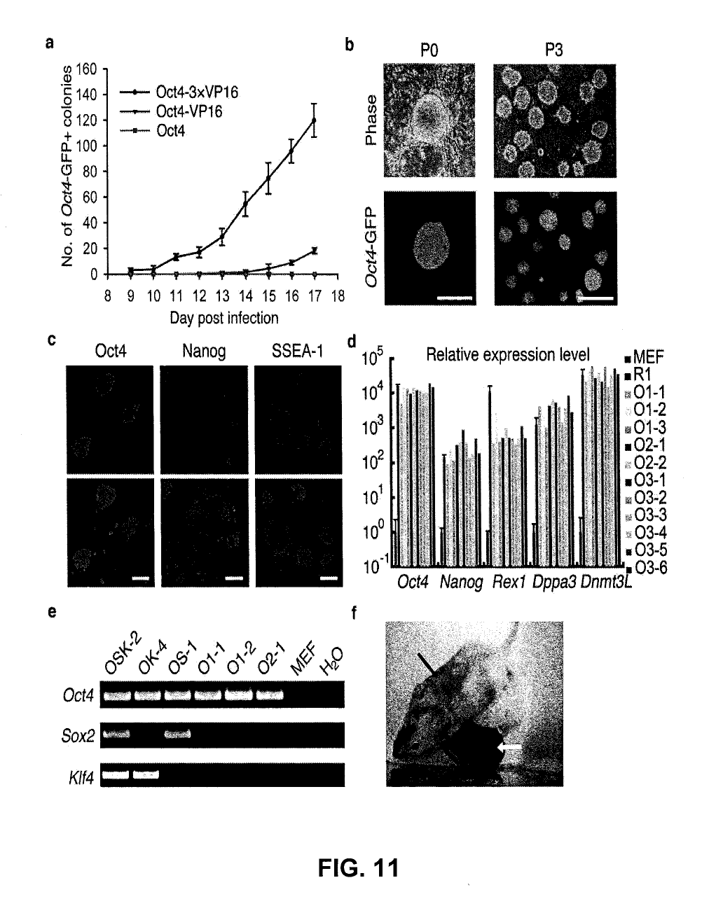

Previous reports show that at least 3 exogenous factors are required to reprogram differentiated somatic cells with very low efficiency (Nakagawa et al., 2008; Wernig et al., 2008), addition of small molecule compounds can replace certain factors but the efficiency is still very low (Huangfu et al., 2008; Ichida et al., 2009; Li et al., 2010; Lyssiotis et al., 2009; Shi et al., 2008). Because synthetic factors can greatly improve the reprogramming efficiency, we try to use only one synthetic factor Oct4-VP16 to reprogram MEF cells. The 17^{th} day after 5×10⁴ MEF cells were infected with Oct4-VP16 retrovirus, average 18 GFP-positive iPS clones appear (FIG. 11a, b). The results also show that, among synthetic factors, the copy number of VP16 greatly increases and the process of reprogramming accelerates. Use of a single factor with Oct4 fused with 3 VP16 to induce reprogramming, GFP positive clones appeared on the 9^{th} day after viral infection. This number increases to 120 in the 17th day (FIG. 11a), up to about 0.24% of the reprogramming efficiency. The efficiency of this and OKS three-factor (Nakagawa et al., 2008; Wernig et al., 2008) or OKSM four-factor (Okita et al., 2007; Wernig et al., 2007) are even higher. The iPS cells established by Oct4-VP16 single factor express Oct4, Nanog, SSEA-1 (FIG. 11c) and other totipotency genes (FIG. 11d). These iPS cells, as confirmed by PCR, contain only Oct4-VP16 transgenic (FIG. 11e), and can produce chimeric mice having the capacity of reproductive transmission (FIG. 11f). These results demonstrated, for the first time, that only one factor is required to reprogram MEF cells with high efficiency.

6. Through methods of episomal plasmid introduction, use of synthetic factors can efficiently produce iPS cells without DNA insertion.

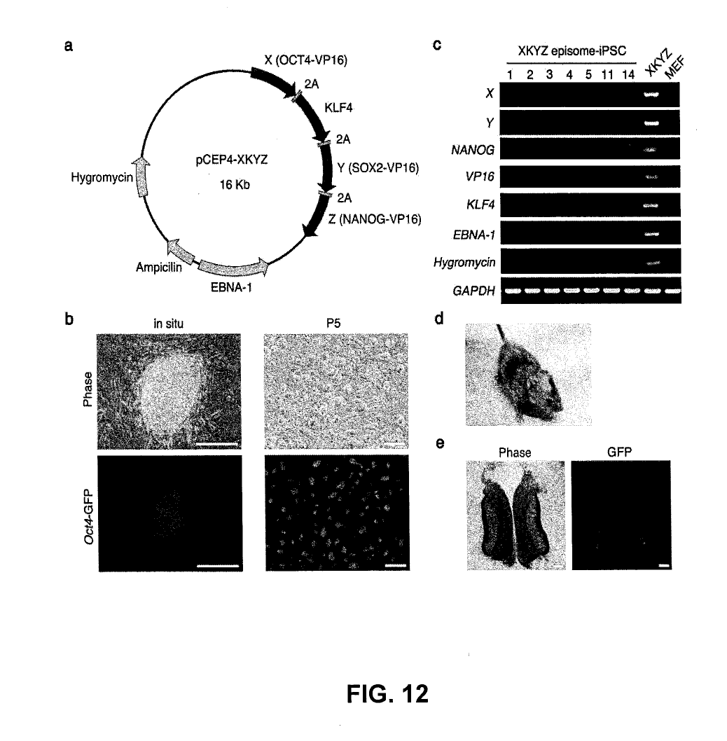

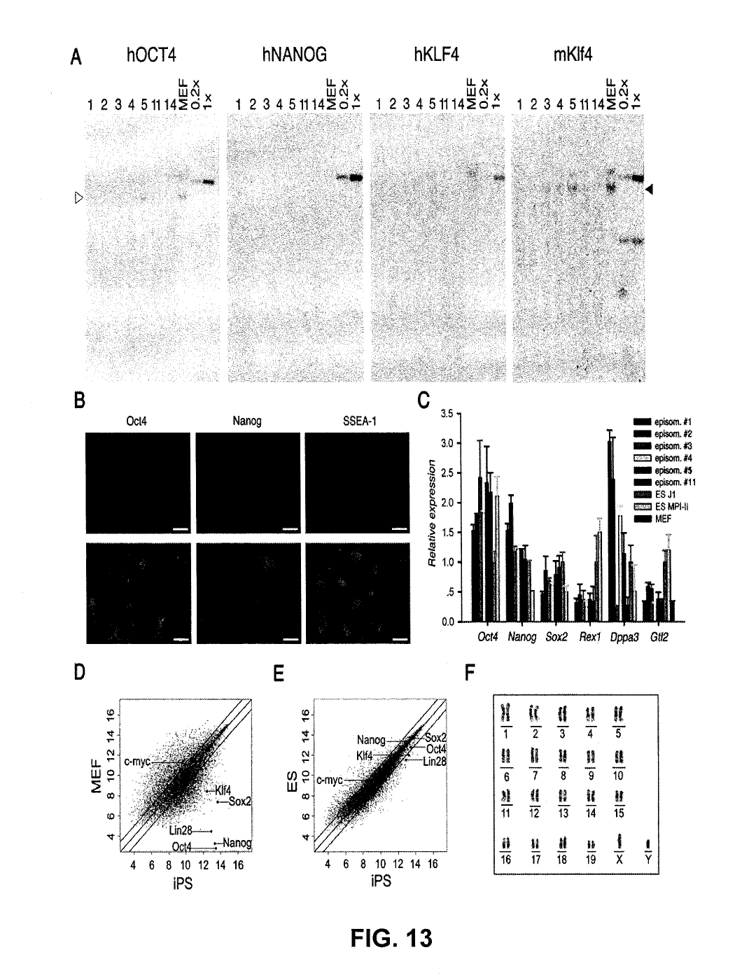

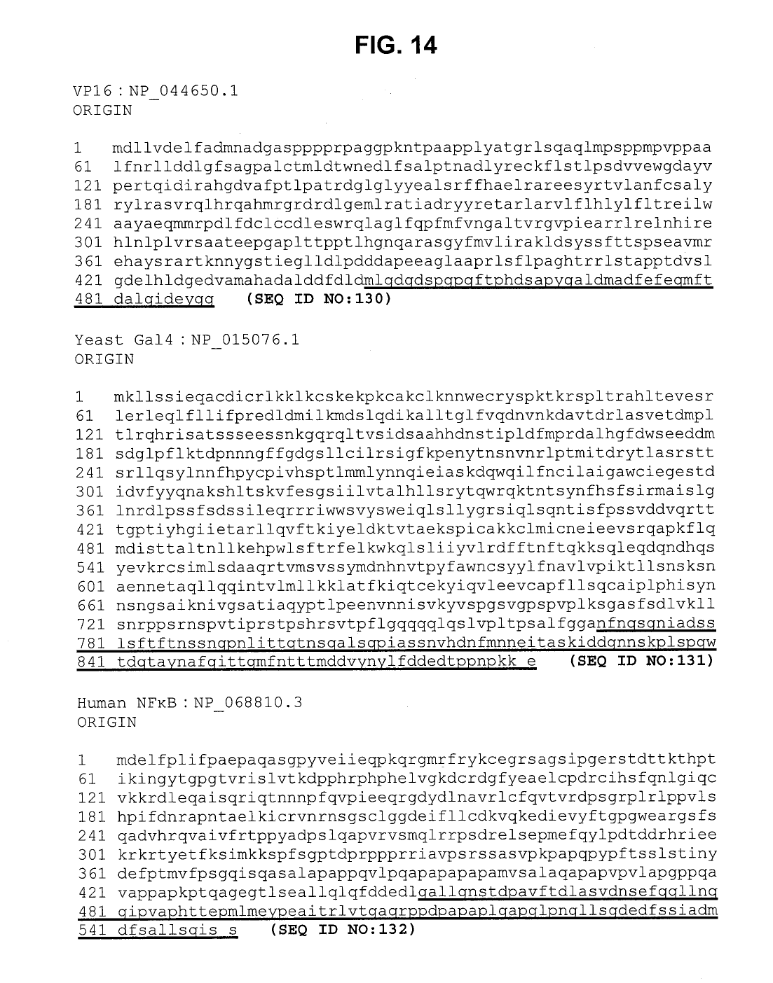

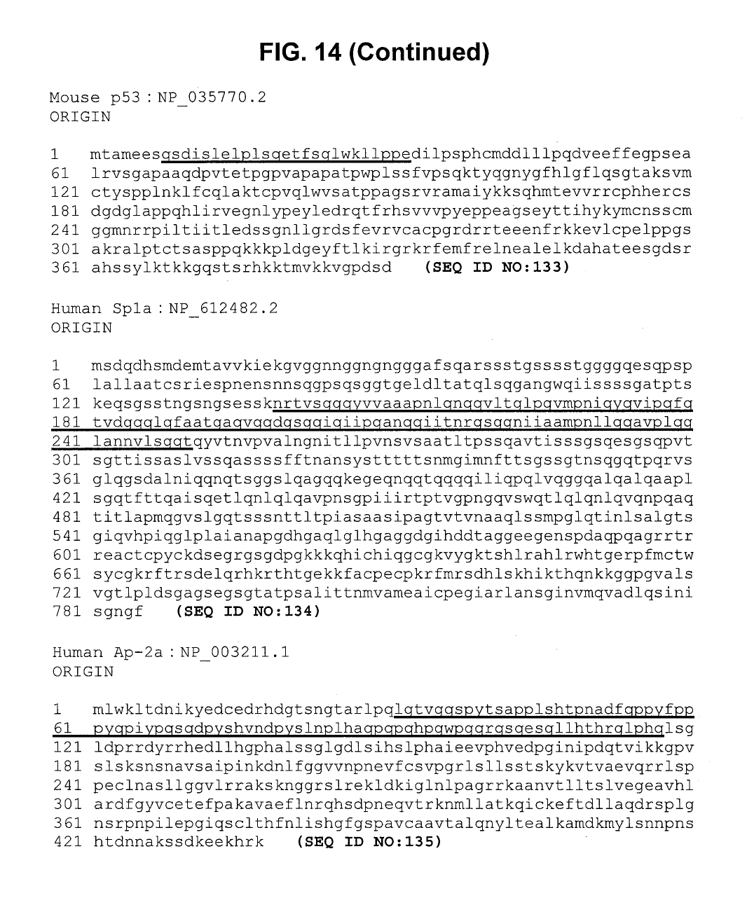

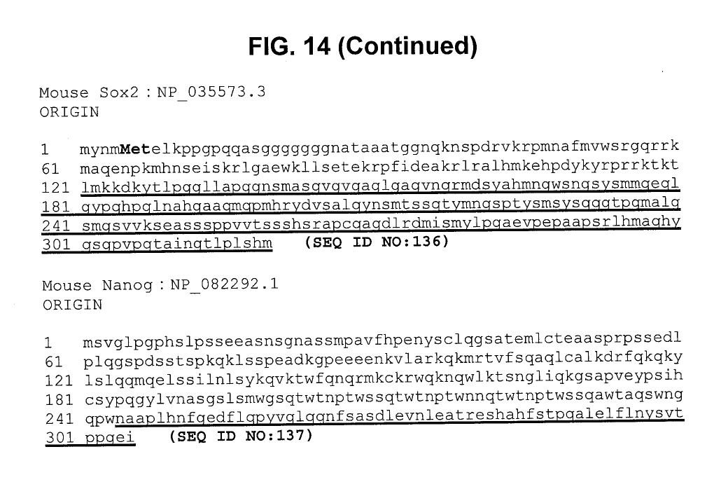

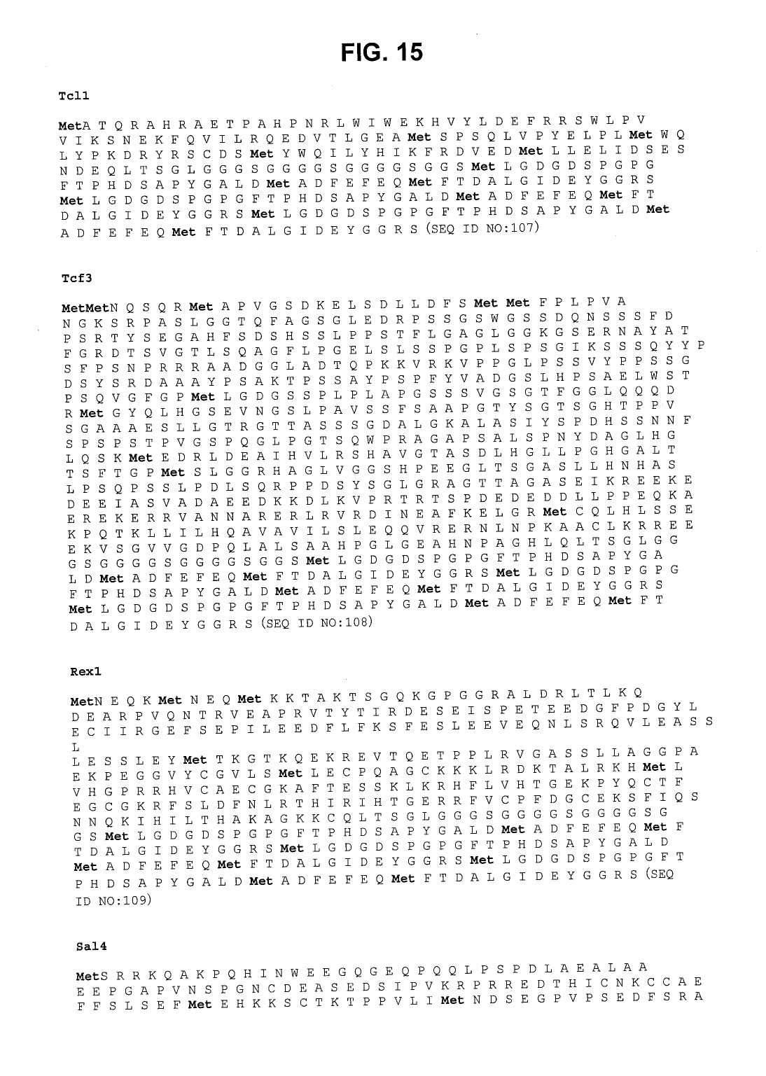

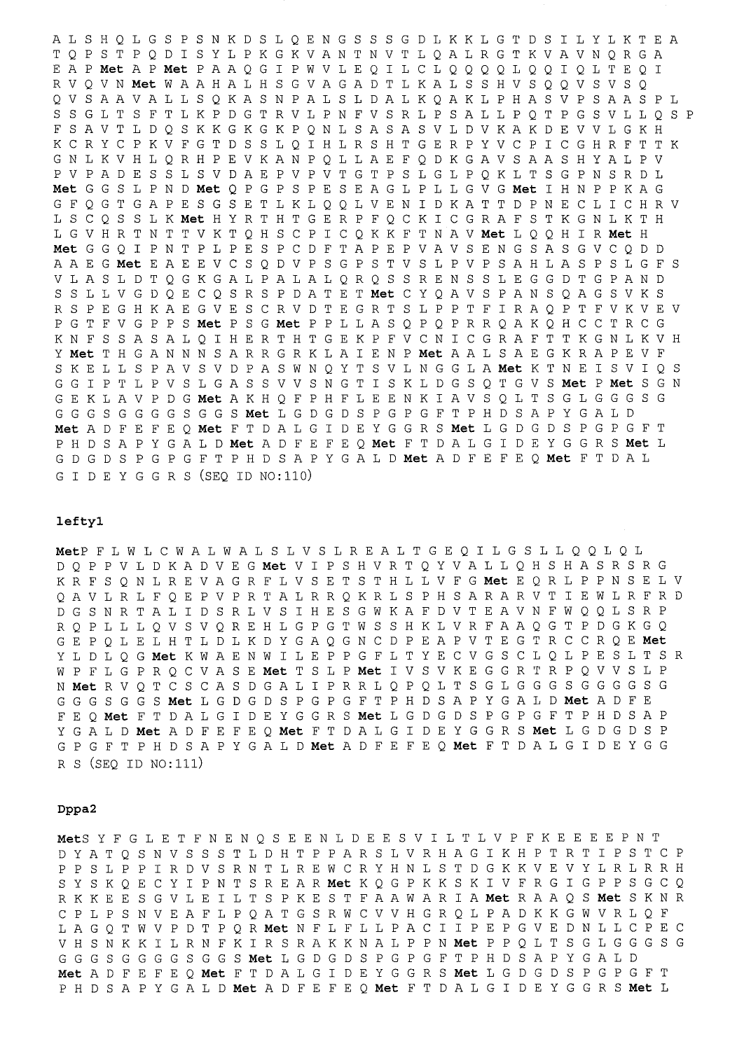

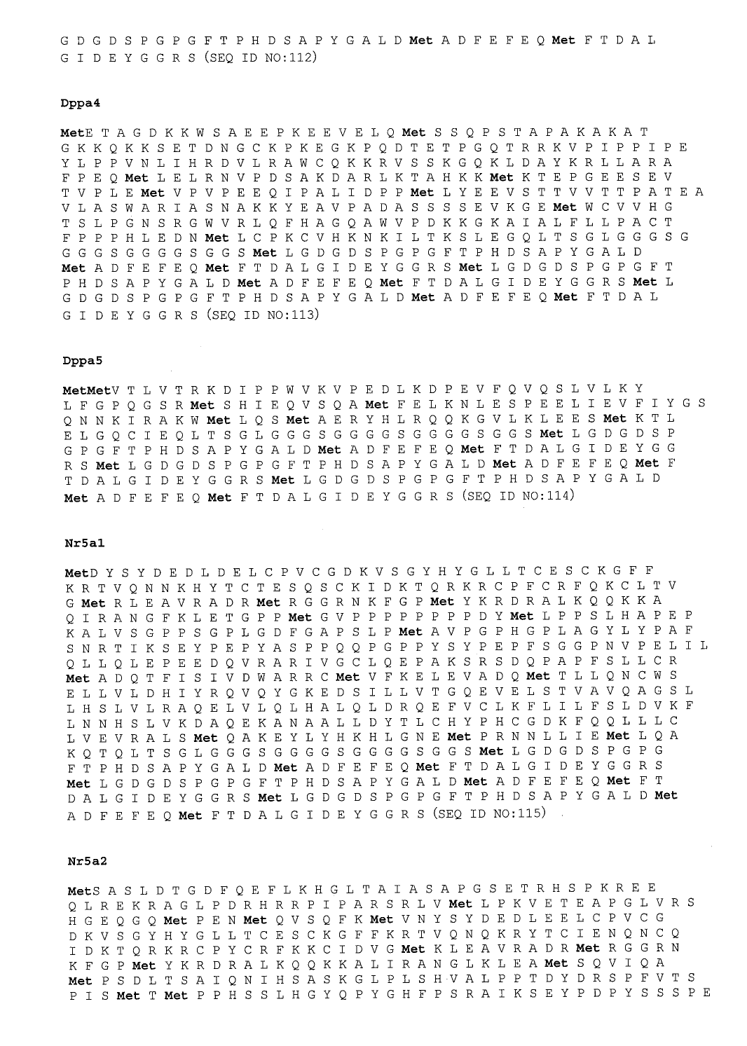

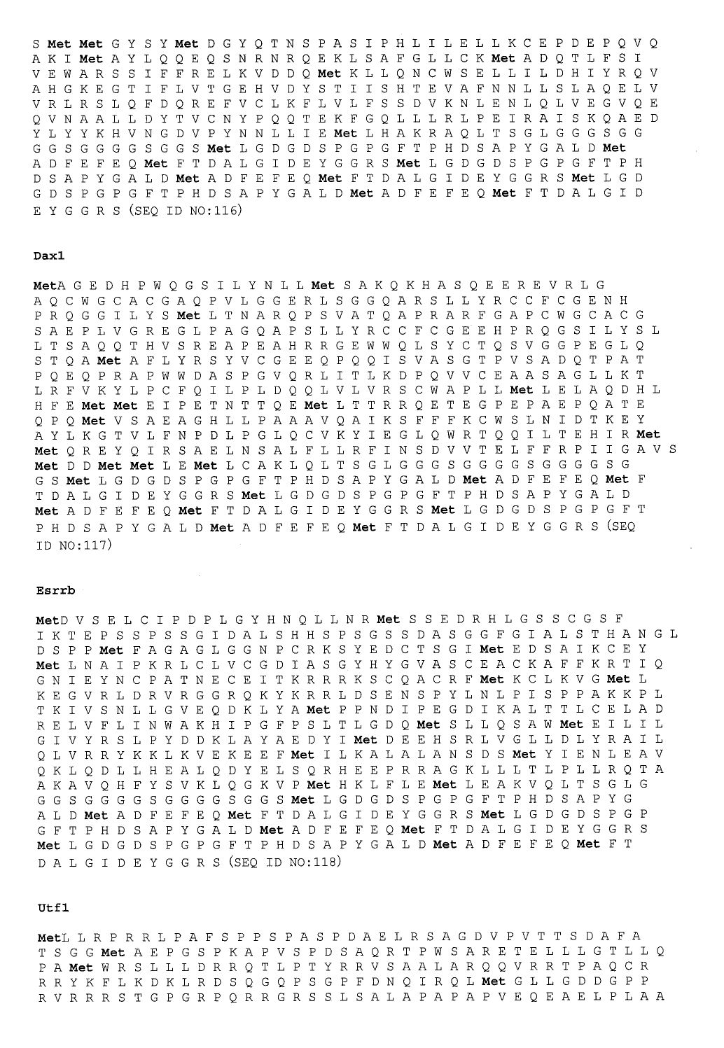

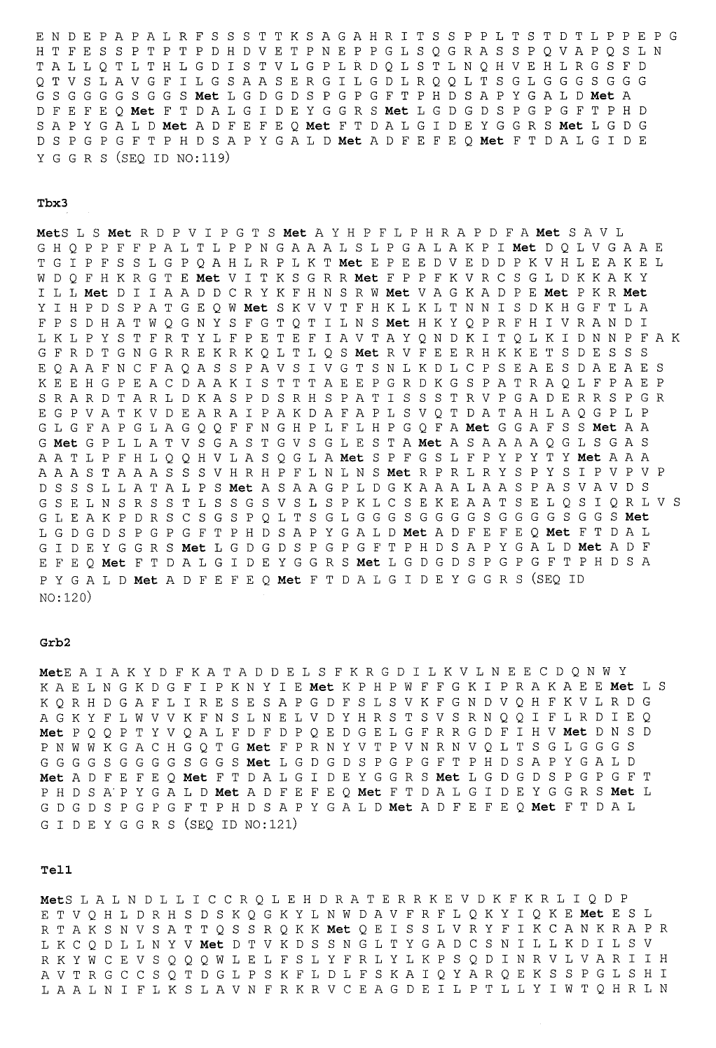

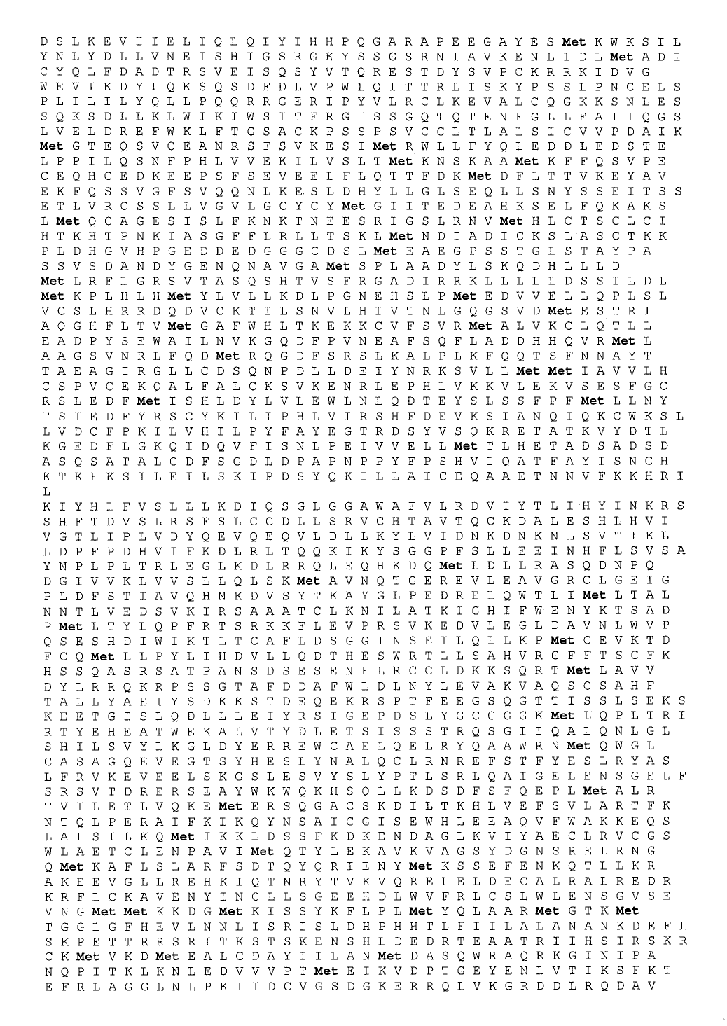

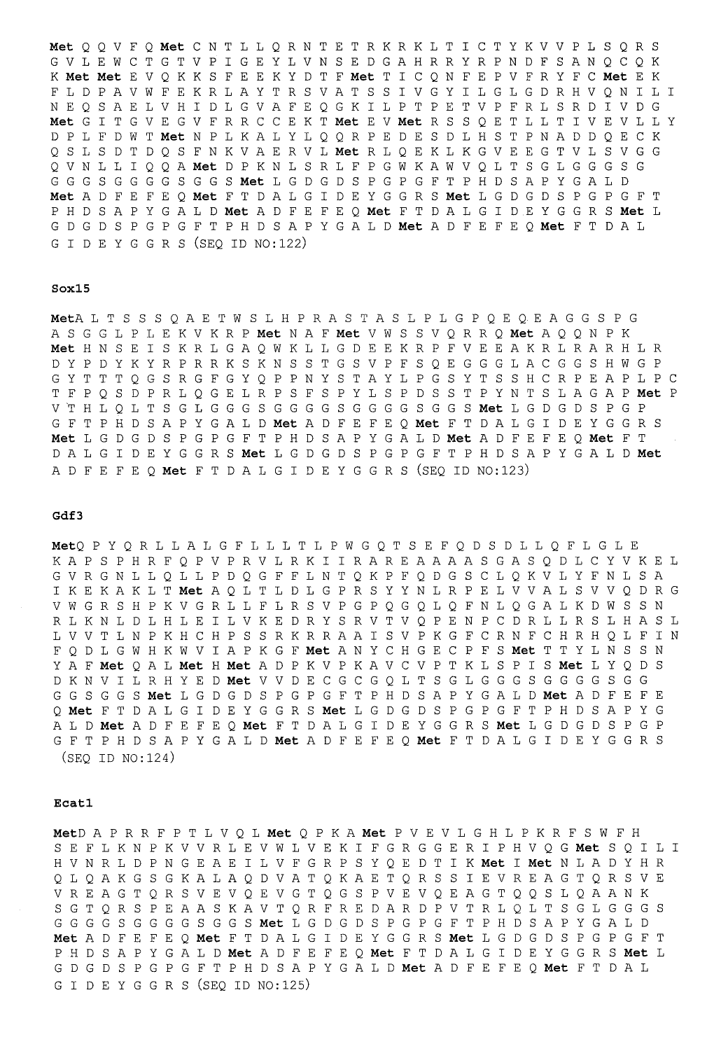

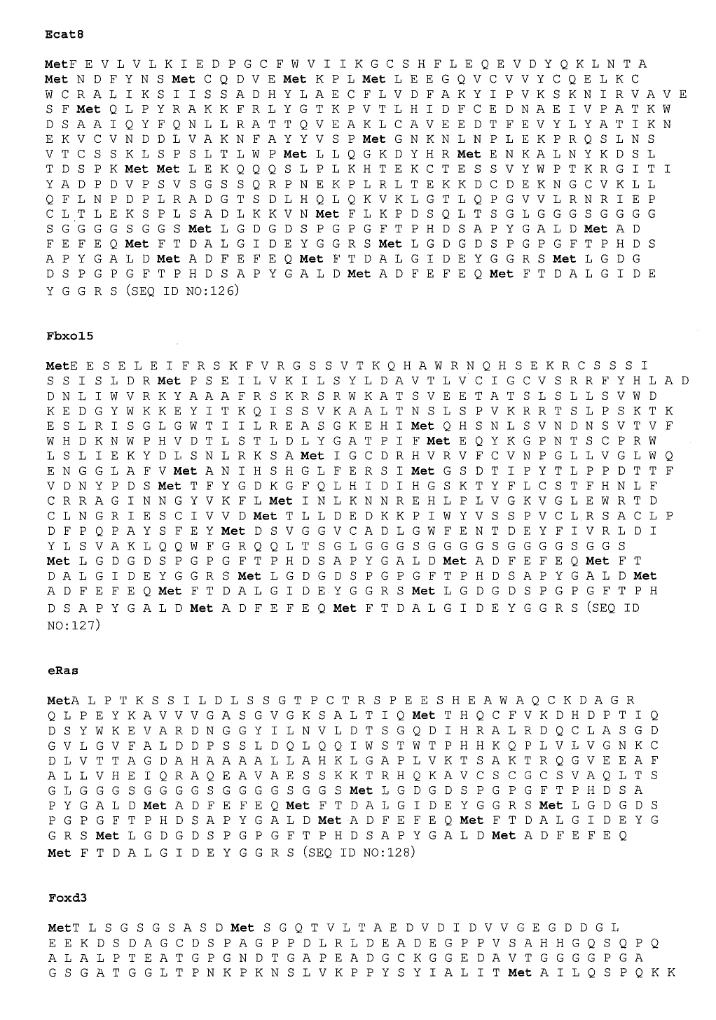

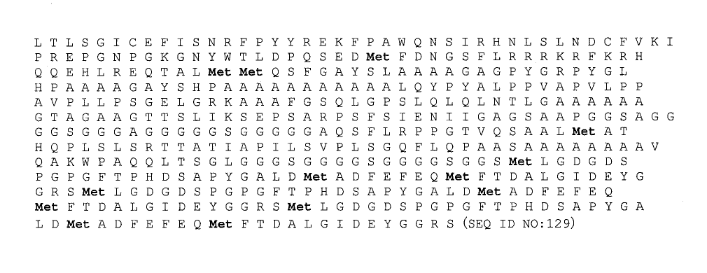

It was very difficult to produce iPS cells without DNA insertion. We have successfully accomplished the attempt to carry out reprogramming by introducing synthetic factors through a plasmid into MEF cells. After sequentially connecting coding sequences of OCT4-VP16, KLF4, SOX2-VP16 and NANOG-VP16 in series through 2A elements, it is cloned into vector pCEP4 (Invitrogen) to generate pCEP4-XKYZ plasmid (FIG. 12a). 18 days after transfecting pCEP4-XKYZ into 1×10⁶ MEF cells through electric shock, we observed 55-450 Oct4-GFP positive iPS clones. We randomly selected 24 iPS clones and confirm that they can establish stable cell lines (FIG. 12b). Through PCR testing on genomic DNA, we found that these cell lines do not contain plasmid DNA insertion (FIG. 12c). Southern hybridization using specific probes for transgenic also demonstrate that these cells do not contain plasmid DNA insertion (FIG. 13a). Further immunofluorescence (FIG. 13b), quantitative PCR (FIG. 13c) and genomic gene expression profile analysis (FIG. 13d, e) show iPS cells are very close to ES cells. These iPS cells have normal karyotype (FIG. 13f) and can produce chimeric mice, which have the ability to incorporate into reproductive system (FIG. 12d, e). In prior reports, the work of preparing iPS cells without DNA insertion requires the use of c-Myc oncogene with very low efficiency (Kim et al., 2009a; Okita et al., 2008; Yu et al., 2009; Zhou et al., 2009). We demonstrated for the first time, without using c-Myc, the efficiency reaches about 0.03% by using synthetic factors.

7. Reprogramming factors, when fused with structural domains having transcription activation function, can enhance the reprogramming ability (such as Oct4 fused with the transcription activator domain of VP16, Gal4, p53, NFκB, Sp1, AP2 and Nanog).

We use the reprogram experimental system of mouse MEF cells containing Oct4-GFP reporter gene to test the effects of synthetic factors generated by fusing Oct4 with a series of transcription activator domains of transcription factors on reprogramming (see Table 3). These transcription activator domains include various types of domains that are rich in acidic amino acids, glutamine, proline, and serine/threonine, etc. They belong to a wide spectrum of species, including herpes simplex virus, yeast, mouse and human. Our experimental results show that, as long as Oct4 is fused with a domain having a transcription activator function, the resulting synthetic factor, as compared with Oct4, would have a significantly increased reprogramming efficiency. Furthermore, the extent of efficiency enhancement increases with the increased transcription activation ability of the fused domain. In addition, we also found that the reprogramming efficiency is greatly enhanced as long as Oct4 is fused in series with 2 fragments, each containing a short peptide of only 12 amino acids (DALDDFDLDMLG) derived from VP16.

8. Part of the reprogramming factors, which contain regions of DNA binding structural domains, fused with stronger transcription activator proteins can induce the production of iPS cells. For example, similar to the intact Oct4, a portion of Oct4 fused with VP16 transcription activator domain can be used for reprogramming (see SEQ ID NO:90 and 91).

TABLE 3

| Fusion Elements | | | | | |
|---|---|---|---|---|---|
| | | Transcription | | | |
| | | regulatory | | Effect on | Nucleotide/amino |
| Transcription | Linker | sequence and | Labeling | iPS colony | acid sequence No. |
| factor | sequence | position | position | formation | (SEQ ID NO:) |
| GCNF (1-266) | RS | VP16 | C | No | |
| RARα (1-167) | | AD (446-490), | | No | |
| PPARγ (31-183) | | C terminal | | No | |
| SF-1 (1-96) | | | | No | |
| LRH-1 (1-129) | | | | No | |
| Nanog | | | | + | 73/76 |
| Oct4 | G(SGGGG)₂SG | VP16 | N | ++ | 77/92 |
| Sox2 | GGLGSTEF | AD (446-490), | | + | 78/93 |
| Nanog | | N terminus | | + | 79/94 |
| Oct4 | RSTSGLGGGS | VP16 | C | ++ | 71/74 |
| Sox2 | (GGGGS)₂G | AD (446-490), | | + | 72/75 |
| Nanog | | C terminus | | + | 80/95 |
| Klf4 | | | | No | |
| Oct4 | QLTSGLGGGS | three serially | N | +++ | 81/96 |
| | (GGGGS)₂G | connected VP16 | | | |
| | | AD (446-490), | | | |
| | | C terminus | | | |
| | | two serially | | ++ | 82/97 |
| | | connected VP16 | | | |
| | | AD (437-448), | | | |
| | | C terminaus | | | |
| | | yeast Gal4 | | +++++ | 83/98 |
| | | AD (768-881), | | | |
| | | C terminus | | | |
| Oct4 | QLTSGLGGGS | human NFκB | N | +++++ | 84/99 |
| | (GGGGS)₂G | AD (451-551), | | | |
| | | C terminus | | | |
| | | mouse p53 | | + | 85/100 |
| | | TAD (8-32), | | | |
| | | C terminus | | | |
| | | human Sp1a | | + | 86/101 |
| | | AD (139-250), | | | |
| | | C terminus | | | |
| | | human Ap-2a | | + | 87/102 |
| | | AD (31-117), | | | |
| | | C terminus | | | |
| | | mouse Sox2 | | + | 88/103 |
| | | AD (121-319), | | | |
| | | C terminus | | | |
| | | mouse Nanog | | + | 89/104 |
| | | AD (244-305), | | | |
| | | C terminus | | | |
| Klf4 | | Engrailed | | − | |
| | | repressor | | | |
| Oct4 | | (2-298), | | − | |
| | | C terminus | | | |
| Oct4 (127-352) | G(SGGGG)₂SG | VP16 | N | + | 90/105 |
| | GGLGSTEF | AD (446-490), | | | |
| | | N terminus | | | |
| Oct4 (1-286) | RSTSGLGGGS | VP16 | C | + | 91/106 |
| | (GGGGS)₂G | AD (446-490), | | | |
| | | C terminus | | | |

In the table, “+” indicates having stimulatory effects; “−” indicates having inhibitory effects.

GCNF, RAR α, PPAR γ, SF-1 and LRH-1 in the Table are, as reported, proteins that can bind to Oct4 promoter regions and can regulate its expression. We selected the portions having DNA binding activity of these proteins and fused them with VP16 AD.

We show that synthetic factors with enhanced transcription activation ability can promote the establishment of ES transcriptional regulatory network to reactivate endogenous totipotency genes including Oct4, Sox2 and genes of other purposes. These reactivated endogenous factors may contribute to further reprogramming and ultimately improve the efficiency of iPS cell generation.

Recent reports indicate that p53 signal pathway inhibits the potential of cellular replication; inhibiting p53 signaling pathway can significantly improve the efficiency of iPS cell generation (Zhao, Y. et al. Two supporting factors greatly improve the efficiency of human iPSC generation. Cell Stem Cell 3, 475-9 (2008); Hong, H. et al. Suppression of induced pluripotent stem cell generation by the p53-p21 pathway. Nature (2009); Utikal, J. et al. Immortalization eliminates a roadblock during cellular reprogramming into iPS cells. Nature 460, 1145-8 (2009); Marion, R. M. et al. A p53-mediated DNA damage response limits reprogramming to ensure iPS cell genomic integrity. Nature 460, 1149-53 (2009); Li, H. et al. The Ink4/Arf locus is a barrier for iPS cell reprogramming. Nature 460, 1136-9 (2009); Kawamura, T. et al. Linking the p53 tumour suppressor pathway to somatic cell reprogramming. Nature 460, 1140-4 (2009)). We found that synthetic factors do not increase their reprogramming ability through decreasing the expression levels of p53. Experimental results show that the expression levels of p53 are actually un-regulated in the transfected MEF cells (FIG. S6). iPS cells generated by synthetic factors would not harm genome stability due to p53 inactivation, thus, cancer risk does not exist. Tumors formed from induced pluripotent stem cells generated by using synthetic factors do not increase, as compared with the induced pluripotent stem cells generated by natural factors, the tumor occurrence rates do not increase.

Different from the above, we show that synthetic factors increase reprogramming efficiency by accelerating two major rate-limiting steps: DNA demethylation and reactivation of pluripotency genes (FIG. 4f). In somatic cells, Nanog and Oct4 promoters are stably silenced by DNA methylation (Li, J. Y. et al. Synergistic function of DNA methyltransferases Dnmt3a and Dnmt3b in the methylation of Oct4 and Nanog. Mol Cell Biol 27, 8748-59 (2007)). Consistent with this, there is no detectable levels of Oct4-GFP expression in the first few days, even though MEF cells express high levels of Oct4-VP16 and other factors. This shows that the inactive chromatins containing inhibitory markers cause difficulty for transcription factors to reach gene promoter regions, and that seriously impede transcription initiation. VP16, after fused with Oct4, Nanog and Sox2, reactivates target genes via certain unknown mechanisms to accelerate the removal of inhibitory markers. Our findings support this concept: a wide range of DNA demethylation occurs in Oct4 promoter regions in GFP positive cells selected in different periods (FIG. S4).

It is known that exogenous expression of Nanog-VP16 in mouse ES cells causes ES cells to differentiate (Wang, Z., Ma, T., Chi, X. & Pei, D. Aromatic residues in the C-terminal domain 2 are required for Nanog to mediate LIF-independent self-renewal of mouse embryonic stem cells. J Biol Chem 283, 4480-9 (2008)). However, in our systems, this kind of harmful effects may be avoided because endogenous Dnmt3L and Dnmt3a2 reactivation in the reprogrammed cells can start DNA methylation in the retroviral promoter regions. Nevertheless, the duration for exogenous expression of synthetic factors needs to be tested to optimize iPS cell reprogramming. In addition, synthetic factors can improve the efficiency of synthetic factors by methods of increasing their transcription activation activity, protein stability, and intracellular localization. For example, improvement can be achieved by fusing Oct4 and three serially connected VP16 or Oct4 mutants with ubiquitination sites removed to resist protein degradation mediated by proteasomes (Xu, H. et al. WWP2 promotes degradation of transcription factor OCT4 in human embryonic stem cells. Cell Res 19, 561-73 (2009)). When reprogramming factors are introduced into cells by non-viral methods, their cellular concentrations should be at a relatively low level, using enhanced transcription factors then becomes important. We obtain very high efficiency and reproducibility in producing iPS cells using plasmid transient transfection of synthetic factors. A wide range of prospective applications remains for the modified synthetic factors to be used as functional cells in cell reprogramming including directional differentiation of stem cells and generation of precursor cells.

1. A fusion protein, characterized in that the fusion protein comprises
a protein encoded by a gene related to cell totipotency or a fragment thereof, and
a transcription regulatory domain or a fragment thereof having transcription regulatory activity.

2. The fusion protein according to claim 1, characterized in that the gene related to cell totipotency is selected from OCT4, NANOG, SOX2, Tcl1, Tcf3, Rex1, Sal4, lefty1, Dppa2, Dppa4, Dppa5, Nr5a1, Nr5a2, Dax1, Esrrb, Utf1, Tbx3, Grb2, Tel1, Sox15, Gdf3, Ecat1, Ecat8, Fbxo15, eRas, or Foxd3.

3. The fusion protein according to claim 1, characterized in that the gene related to cell totipotency is selected from Oct4, NANOG, or SOX2.

4. The fusion protein according to claim 1 characterized in that the protein encoded by the gene related to cell totipotency is selected from the amino acid sequence at positions127-352 of Oct4 or the amino acid sequence at positions 1-286 of Oct4.

5. The fusion protein according to claim 1, characterized in that the transcription regulatory domain is selected from a transcription regulatory domain of viral protein VP16, EBNA2, and E1A or a fragment thereof having transcription activity; or selected from a transcription regulatory domain of yeast Gal4, Oaf1, Leu3, Rtg3, Pho4, Gln3, Gcn4, Gli3, Pip2, Pdr1, Pdr3, Lac9, and Tea1 or a fragment thereof having transcription activity; or selected from a transcription regulatory domain of mammalian p53, NFAT, Sp1 (e.g., Sp1a), AP-2 (e.g., Ap-2a), Sox2, NF-κB, MLL/ALL, E2A, CREB, ATF, /JUN, FOS HSF1, KLF2, NF-IL6, ESX, Oct1, Oct2, SMAD, CTF, HOX, Sox2, Sox4, and Nanog or a fragment thereof having transcription activity; or selected from a transcription regulatory domain of plant HSF or a fragment thereof having transcription activity.

6. The fusion protein according to claim 1, characterized in that the transcription regulatory domain is selected from a transcription regulatory domain of viral protein VP16 or a fragment thereof having transcription activity, or selected from a transcription regulatory domain of yeast Gal4 or a fragment thereof having transcription activity, or selected from a transcription regulatory domain of mammalian p53, Sp1a, Ap-2a, Sox2, NF-κB, and Nanog or a fragment thereof having transcription activity.

7. The fusion protein according to claim 1, characterized in that the transcription regulatory domain is selected from: the amino acid sequence at positions 446-490 of VP16, the amino acid sequence at positions 437-448 of VP16, the amino acid sequence at positions 768-881 of yeast Gal4, the amino acid sequence at positions 451-551 of NFκB, the amino acid sequence at positions 8-32 of mouse p53, the amino acid sequence at positions 139-250 of Sp1a, the amino acid sequence at positions 31-117 of Ap-2a, the amino acid sequence at positions 121-319 of mouse Sox2, and the amino acid sequence at positions 244-305 of mouse Nanog.

8. The fusion protein according to claim 1, characterized in that the fusion protein comprises one or more transcription regulatory domains, which are the same or different.

9. The fusion protein according to claim 1, characterized in that the fusion protein is one selected from the amino acid sequences of SEQ ID NO:74-76 and 92-129.

10. A nucleotide sequence, characterized in that the nucleotide sequence encodes the fusion protein according to claim 1.

11. The nucleotide sequence according to claim 10, characterized in that the nucleotide sequence is one selected from SEQ ID NO:71, SEQ ID NO:72, SEQ ID NO:73, and SEQ ID NO:77-91.

12. An expression vector, characterized in that the expression vector comprises the nucleotide sequence according to claim 10.

13. A composition, characterized in that the composition comprises the fusion protein according to claim 1 and a carrier or an excipient.

14. The composition according to claim 13, characterized in that the composition comprises at least one selected from the following fusion proteins: a fusion protein formed of OCT4 protein and a transcriptional regulatory domain of VP16 protein encoded by herpes simplex virus, a fusion protein formed of NANOG and a transcriptional regulatory domain of VP16 protein encoded by herpes simplex virus, a fusion protein formed of SOX2 protein and a transcriptional regulatory domain of VP16 protein encoded by herpes simplex virus, and a fusion protein formed of Oct4 and a transcriptional regulatory domain of yeast Gal4, or human NFκB, or mouse p53, or human Sp1a, or human Ap-2a, or mouse Sox2, or mouse Nanog.

15. A method for reprogramming a somatic cell to become an induced pluripotent stem cell or a cell of other lineage with a different function, characterized in that the method comprises:
(1) treating the somatic cell with the nucleotide sequence according to claim 10, and
(2) after culturing the treated cells, screening for cells with pluripotent stem cell characteristics or cells of other lineage with a different function to obtain the induced pluripotent stem cell or the cell of other lineage with a different function.

16. The method according to claim 15, characterized in that the method comprises introducing the fusion protein, the nucleotide sequence, the expression vector, and/or the composition into the somatic cell by viral infection, plasmid transfection, protein transduction, and/or mRNA transfection.

17. A cell containing the fusion protein according to claim 1.

18. The cell according to claim 17, characterized in that the cell is an induced pluripotent stem cell or a cell different from the original cell.

19. A composition, characterized in that the composition comprises the nucleotide sequence according to claim 10 and a carrier or an excipient.

20. A composition, characterized in that the composition comprises the expression vector according to claim 12 and a carrier or an excipient.


SEQUENCE LISTING (137 sequences)

SEQ ID NO: 1 - DNA, 23 bp, Artificial Sequence
  Primer
tctttccaccaggcccccggctc

SEQ ID NO: 2 - DNA, 23 bp, Artificial Sequence
  Primer
tgcgggcggacatggggagatcc

SEQ ID NO: 3 - DNA, 23 bp, Artificial Sequence
  Primer
tagagctagactccgggcgatga

SEQ ID NO: 4 - DNA, 23 bp, Artificial Sequence
  Primer
ttgccttaaacaagaccacgaaa

SEQ ID NO: 5 - DNA, 24 bp, Artificial Sequence
  Primer
taggctgatttggttggtgtcttg

SEQ ID NO: 6 - DNA, 20 bp, Artificial Sequence
  Primer
agtgtgatggcgagggaagg

SEQ ID NO: 7 - DNA, 18 bp, Artificial Sequence
  Primer
agaagaacggcatcaagg

SEQ ID NO: 8 - DNA, 18 bp, Artificial Sequence
  Primer
gctcaggtagtggttgtc

SEQ ID NO: 9 - DNA, 24 bp, Artificial Sequence
  Primer
gaagtctggttccttggcaggatg

SEQ ID NO: 10 - DNA, 20 bp, Artificial Sequence
  Primer
actcgatacactggcctagc

SEQ ID NO: 11 - DNA, 24 bp, Artificial Sequence
  Primer
tgctgcggtccaggccatcaagag

SEQ ID NO: 12 - DNA, 24 bp, Artificial Sequence
  Primer
gggcactgttcagttcagcggatc

SEQ ID NO: 13 - DNA, 24 bp, Artificial Sequence
  Primer
actgcccctcatcagactgctact

SEQ ID NO: 14 - DNA, 24 bp, Artificial Sequence
  Primer
cactgccttgtactcgggtagctg

SEQ ID NO: 15 - DNA, 24 bp, Artificial Sequence
  Primer
acgagtggcagtttcttcttggga

SEQ ID NO: 16 - DNA, 24 bp, Artificial Sequence
  Primer
tatgactcacttccagggggcact

SEQ ID NO: 17 - DNA, 24 bp, Artificial Sequence
  Primer
ccattaggggccatcatcgctttc

SEQ ID NO: 18 - DNA, 24 bp, Artificial Sequence
  Primer
cactgctcactggagggggcttgc

SEQ ID NO: 19 - DNA, 28 bp, Artificial Sequence
  Primer
tgtggggccctgaaaggcgagctgagat

SEQ ID NO: 20 - DNA, 28 bp, Artificial Sequence
  Primer
atgggccgccatacgacgacgctcaact

SEQ ID NO: 21 - DNA, 20 bp, Artificial Sequence
  Primer
agaaggtgaccagcctgaca

SEQ ID NO: 22 - DNA, 20 bp, Artificial Sequence
  Primer
gttctgaaccagcaggctta

SEQ ID NO: 23 - DNA, 25 bp, Artificial Sequence
  Primer
ctcacacctgagctgtactgcagag

SEQ ID NO: 24 - DNA, 25 bp, Artificial Sequence
  Primer
ctccaccttctgagactctccagag

SEQ ID NO: 25 - DNA, 26 bp, Artificial Sequence
  Primer
ttcagtgaccagtcctcagacacgaa

SEQ ID NO: 26 - DNA, 25 bp, Artificial Sequence
  Primer
tcagaaggctggagacctccctctt

SEQ ID NO: 27 - DNA, 24 bp, Artificial Sequence
  Primer
gtgcgggtactgagcctttttaga

SEQ ID NO: 28 - DNA, 25 bp, Artificial Sequence
  Primer
cgacatttgtgacatcttccacgta

SEQ ID NO: 29 - DNA, 18 bp, Artificial Sequence
  Primer
gtgtgcatgacgtgcggg

SEQ ID NO: 30 - DNA, 22 bp, Artificial Sequence
  Primer
gcagttcgaatctgcaccgtag

SEQ ID NO: 31 - DNA, 21 bp, Artificial Sequence
  Primer
gctctggctttcgtgaacatg

SEQ ID NO: 32 - DNA, 22 bp, Artificial Sequence
  Primer
tcgaatctgcaccgtagttgag

SEQ ID NO: 33 - DNA, 22 bp, Artificial Sequence
  Primer
agtcaaggccgagaatgggaag

SEQ ID NO: 34 - DNA, 22 bp, Artificial Sequence
  Primer
aagcagttggtggtgcaggatg

SEQ ID NO: 35 - DNA, 20 bp, Artificial Sequence
  Primer
tctcccatgcattcaaactg

SEQ ID NO: 36 - DNA, 20 bp, Artificial Sequence
  Primer
cttttattttatcgtcgacc

SEQ ID NO: 37 - DNA, 20 bp, Artificial Sequence
  Primer
ctgcccctgtcgcacatgtg

SEQ ID NO: 38 - DNA, 20 bp, Artificial Sequence
  Primer
cttttattttatcgtcgacc

SEQ ID NO: 39 - DNA, 19 bp, Artificial Sequence
  Primer
catcgcagcttggatacac

SEQ ID NO: 40 - DNA, 18 bp, Artificial Sequence
  Primer
gcattgatgaggcgttcc

SEQ ID NO: 41 - DNA, 20 bp, Artificial Sequence
  Primer
ccttacacatgaagaggcac

SEQ ID NO: 42 - DNA, 20 bp, Artificial Sequence
  Primer
cttttattttatcgtcgacc

SEQ ID NO: 43 - DNA, 22 bp, Artificial Sequence
  Primer
gaaatcgtgcgtgacatcaaag

SEQ ID NO: 44 - DNA, 22 bp, Artificial Sequence
  Primer
tgtagtttcatggatgccacag

SEQ ID NO: 45 - DNA, 24 bp, Artificial Sequence
  Primer
gacagggggaggggaggagctagg

SEQ ID NO: 46 - DNA, 26 bp, Artificial Sequence
  Primer
cttccctccaaccagttgccccaaac

SEQ ID NO: 47 - DNA, 26 bp, Artificial Sequence
  Primer
gggaaatgggaggggtgcaaaagagg

SEQ ID NO: 48 - DNA, 26 bp, Artificial Sequence
  Primer
ttgcgtgagtgtggatgggattggtg

SEQ ID NO: 49 - DNA, 25 bp, Artificial Sequence
  Primer
cagccccgattcttccaccagtccc

SEQ ID NO: 50 - DNA, 25 bp, Artificial Sequence
  Primer
cggaagattcccagtcgggttcacc

SEQ ID NO: 51 - DNA, 24 bp, Artificial Sequence
  Primer
cagatcctaaacagctcgcagaat

SEQ ID NO: 52 - DNA, 23 bp, Artificial Sequence
  Primer
gcgtacgcaaattaaagtccaga

SEQ ID NO: 53 - DNA, 24 bp, Artificial Sequence
  Primer
atatcccgccgtgggtgaaagttc

SEQ ID NO: 54 - DNA, 24 bp, Artificial Sequence
  Primer
actcagccatggactggagcatcc

SEQ ID NO: 55 - DNA, 24 bp, Artificial Sequence
  Primer
cttatgctacgtaaaggagctggg

SEQ ID NO: 56 - DNA, 24 bp, Artificial Sequence
  Primer
gtgccaacccaggtcccggaagtt

SEQ ID NO: 57 - DNA, 24 bp, Artificial Sequence
  Primer
ggagccgcctgccctggaaaattc

SEQ ID NO: 58 - DNA, 24 bp, Artificial Sequence
  Primer
tttttcctgatattctattcccat

SEQ ID NO: 59 - DNA, 24 bp, Artificial Sequence
  Primer
ggagccgcctgccctggaaaattc

SEQ ID NO: 60 - DNA, 24 bp, Artificial Sequence
  Primer
tttttcctgatattctattcccat

SEQ ID NO: 61 - DNA, 21 bp, Artificial Sequence
  Primer
tgttgccatcaatgacccctt

SEQ ID NO: 62 - DNA, 19 bp, Artificial Sequence
  Primer
ctccacgacgtactcagcg

SEQ ID NO: 63 - DNA, 25 bp, Artificial Sequence
  Primer
gaggattggaggtgtaatggttgtt

SEQ ID NO: 64 - DNA, 24 bp, Artificial Sequence
  Primer
ctactaacccatcacccccaccta

SEQ ID NO: 65 - DNA, 31 bp, Artificial Sequence
  Primer
caagctttgggttgaaatattgggtttattt

SEQ ID NO: 66 - DNA, 30 bp, Artificial Sequence
  Primer
cggatccctaaaaccaaatatccaaccata

SEQ ID NO: 67 - DNA, 28 bp, Artificial Sequence
  Primer
aagtatggattaatttattaaggtagtt

SEQ ID NO: 68 - DNA, 26 bp, Artificial Sequence
  Primer
aaaaaacccacactcatatcaatata

SEQ ID NO: 69 - DNA, 28 bp, Artificial Sequence
  Primer
aagtatggattaatttattaaggtagtt

SEQ ID NO: 70 - DNA, 24 bp, Artificial Sequence
  Primer
caaccaaatcaacctatctaaaaa

SEQ ID NO: 71 - DNA, 1284 bp, Artificial Sequence
  Coding sequence for the fusion protein containing OCT4 and VP16 transcriptional activation structural domain
atggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaacagatccactagtggccttggaggtggatctggtggaggtggatcaggtggaggtggatcaggccaattcatgttgggggacggggattccccgggtccgggatttaccccccacgactccgccccctacggcgctctggatatggccgacttcgagtttgagcagatgtttaccgatgcccttggaattgacgagtacggtggggactacaaagacgatgacgacaag

SEQ ID NO: 72 - DNA, 1185 bp, Artificial Sequence
  Coding sequence for the fusion protein containing Sox2 and VP16 transcriptional activation structural domain
atgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgagatccactagtggccttggaggtggatctggtggaggtggatcaggtggaggtggatcaggccaattcatgttgggggacggggattccccgggtccgggatttaccccccacgactccgccccctacggcgctctggatatggccgacttcgagtttgagcagatgtttaccgatgcccttggaattgacgagtacggtggggactacaaagacgatgacgacaag

SEQ ID NO: 73 - DNA, 1113 bp, Artificial Sequence
  Coding sequence for the fusion protein containing Nanog and VP16 transcriptional activation structural domain
atgagtgtgggtcttcctggtccccacagtttgcctagttctgaggaagcatcgaattctgggaacgcctcatcaatgcctgcagtttttcatcccgagaactattcttgcttacaagggtctgctactgagatgctctgcacagaggctgcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggttaagacctggtttcaaaaccaaaggatgaagtgcaagcggtggcagaaaaaccagtggttgaagactagcaatggtctgattcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaataggatctatgttgggggacggggattccccgggtccgggatttaccccccacgactccgccccctacggcgctctggatatggccgacttcgagtttgagcagatgtttaccgatgcccttggaattgacgagtacggtggggaattcgatccaaaaaagaagagaaaggtaatggactacaaagacgatgacgacaag

SEQ ID NO: 74 - protein, 428 aa, Artificial Sequence
  Fusion protein containing OCT4 and VP16 transcriptional activation structural domain
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNRSTSGLGGGSGGGGSGGGGSGQFMLGDGDSPGPGFTPHDSAPYGALDMADFEFEQMFTDALGIDEYGGDYKDDDDK

SEQ ID NO: 75 - protein, 395 aa, Artificial Sequence
  Fusion protein containing Sox2 and VP16 transcriptional activation structural domain
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHMRSTSGLGGGSGGGGSGGGGSGQFMLGDGDSPGPGFTPHDSAPYGALDMADFEFEQMFTDALGIDEYGGDYKDDDDK

SEQ ID NO: 76 - protein, 371 aa, Artificial Sequence
  Fusion protein containing Nanog and VP16 transcriptional activation structural domain
MSVGLPGPHSLPSSEEASNSGNASSMPAVFHPENYSCLQGSATEMLCTEAASPRPSSEDLPLQGSPDSSTSPKQKLSSPEADKGPEEEENKVLARKQKMRTVFSQAQLCALKDRFQKQKYLSLQQMQELSSILNLSYKQVKTWFQNQRMKCKRWQKNQWLKTSNGLIQKGSAPVEYPSIHCSYPQGYLVNASGSLSMWGSQTWTNPTWSSQTWTNPTWNNQTWTNPTWSSQAWTAQSWNGQPWNAAPLHNFGEDFLQPYVQLQQNFSASDLEVNLEATRESHAHFSTPQALELFLNYSVTPPGEIGSMLGDGDSPGPGFTPHDSAPYGALDMADFEFEQMFTDALGIDEYGGEFDPKKKRKVMDYKDDDDK

SEQ ID NO: 77 - DNA, 1296 bp, Artificial Sequence
  Coding sequence for the Fusion protein containing Oct4 and VP16 AD (446-490)
atggactacaaagacgatgacgacaagggaattggaatgttgggggacggggattccccgggtccgggatttaccccccacgactccgccccctacggcgctctggatatggccgacttcgagtttgagcagatgtttaccgatgcccttggaattgacgagtacggtgggggatctggatcaggtggaggtggatcaggtggaggtggatctggaggtggacttggcagtactgaattcatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaac

SEQ ID NO: 78 - DNA, 1197 bp, Artificial Sequence
  Coding sequence for the Fusion protein containing Sox2 and VP16 AD (446-490)
atggactacaaagacgatgacgacaagggaattggaatgttgggggacggggattccccgggtccgggatttaccccccacgactccgccccctacggcgctctggatatggccgacttcgagtttgagcagatgtttaccgatgcccttggaattgacgagtacggtgggggatctggatcaggtggaggtggatcaggtggaggtggatctggaggtggacttggcagtactgaattcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatg

SEQ ID NO: 79 - DNA, 1155 bp, Artificial Sequence
  Coding sequence for the Fusion protein containing Nanog and VP16 AD (446-490)
atggactacaaagacgatgacgacaagggaattggaatgttgggggacggggattccccgggtccgggatttaccccccacgactccgccccctacggcgctctggatatggccgacttcgagtttgagcagatgtttaccgatgcccttggaattgacgagtacggtgggggatctggatcaggtggaggtggatcaggtggaggtggatctggaggtggacttggcagtactgaattgatgagtgtgggtcttcctggtccccacagtttgcctagttctgaggaagcatcgaattctgggaacgcctcatcaatgcctgcagtttttcatcccgagaactattcttgcttacaagggtctgctactgagatgctctgcacagaggctgcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggttaagacctggtttcaaaaccaaaggatgaagtgcaagcggtggcagaaaaaccagtggttgaagactagcaatggtctgattcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaata

SEQ ID NO: 80 - DNA, 1143 bp, Artificial Sequence
  Coding sequence for the Fusion protein containing Nanog and VP16 AD (446-490)
atgagtgtgggtcttcctggtccccacagtttgcctagttctgaggaagcatcgaattctgggaacgcctcatcaatgcctgcagtttttcatcccgagaactattcttgcttacaagggtctgctactgagatgctctgcacagaggctgcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggttaagacctggtttcaaaaccaaaggatgaagtgcaagcggtggcagaaaaaccagtggttgaagactagcaatggtctgattcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaataagatccactagtggccttggaggtggatctggtggaggtggatcaggtggaggtggatcaggccaattcatgttgggggacggggattccccgggtccgggatttaccccccacgactccgccccctacggcgctctggatatggccgacttcgagtttgagcagatgtttaccgatgcccttggaattgacgagtacggtggggactacaaagacgatgacgacaag

SEQ ID NO: 81 - DNA, 1593 bp, Artificial Sequence
  Coding sequence for the Fusion protein containing Oct4 and three VP16 AD (446-490) linked in series
atggactacaaagacgatgacgacaagggatctcaattgttccccatggctggacacctggcttcagacttcgccttctcacccccaccaggtgggggtgatgggtcagcagggctggagccgggctgggtggattctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaaccaattgactagtggccttggaggtggatctggtggaggtggatcaggtggaggtggatcaggcggatccatgttgggggacggggattccccgggtccgggatttaccccccacgactccgccccctacggcgctctggatatggccgacttcgagtttgagcagatgtttaccgatgcccttggaattgacgagtacggtgggagatccatgttgggggacggggattccccgggtccgggatttaccccccacgactccgccccctacggcgctctggatatggccgacttcgagtttgagcagatgtttaccgatgcccttggaattgacgagtacggtgggagatccatgttgggggacggggattccccgggtccgggatttaccccccacgactccgccccctacggcgctctggatatggccgacttcgagtttgagcagatgtttaccgatgcccttggaattgacgagtacggtgggagatct

SEQ ID NO: 82 - DNA, 1248 bp, Artificial Sequence
  Coding sequence for the Fusion protein containing Oct4 and two VP16 AD (437-448) linked in series
atggactacaaagacgatgacgacaagggatctcaattgttccccatggctggacacctggcttcagacttcgccttctcacccccaccaggtgggggtgatgggtcagcagggctggagccgggctgggtggattctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaaccaattgactagtggccttggaggtggatctggtggaggtggatcaggtggaggtggatcaggcggatctgacgcgctagacgatttcgatctggacatgttgggatctgacgcgctagacgatttcgatctggacatgttgggatcc

SEQ ID NO: 83 - DNA, 1509 bp, Artificial Sequence
  Coding sequence for the Fusion protein containing Oct4 and yeast Gal4 AD (768-881)
atggactacaaagacgatgacgacaagggatctcaattgttccccatggctggacacctggcttcagacttcgccttctcacccccaccaggtgggggtgatgggtcagcagggctggagccgggctgggtggattctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaaccaattgactagtggccttggaggtggatctggtggaggtggatcaggtggaggtggatcaggcggatccaattttaatcaaagtgggaatattgctgatagctcattgtccttcactttcactaacagtagcaacggtccgaacctcataacaactcaaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagag

SEQ ID NO: 84 - DNA, 1485 bp, Artificial Sequence
  Coding sequence for the Fusion protein containing Oct4 and human NF?B AD (451-551)
atggactacaaagacgatgacgacaagggatctcaattgttccccatggctggacacctggcttcagacttcgccttctcacccccaccaggtgggggtgatgggtcagcagggctggagccgggctgggtggattctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaaccaattgactagtggccttggaggtggatctggtggaggtggatcaggtggaggtggatcaggcggatccggatcaggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctccagatct

SEQ ID NO: 85 - DNA, 1245 bp, Artificial Sequence
  Coding sequence for the Fusion protein containing Oct4 and mouse p53 TAD (8-32)
atggactacaaagacgatgacgacaagggatctcaattgttccccatggctggacacctggcttcagacttcgccttctcacccccaccaggtgggggtgatgggtcagcagggctggagccgggctgggtggattctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaaccaattgactagtggccttggaggtggatctggtggaggtggatcaggtggaggtggatcaggcggatcccagtcggatatcagcctcgagctccctctgagccaggagacattttcaggcttatggaaactacttcctccagaa

SEQ ID NO: 86 - DNA, 1512 bp, Artificial Sequence
  Coding sequence for the Fusion protein containing Oct4 and human Sp1a AD (139-250)
atggactacaaagacgatgacgacaagggatctcaattgttccccatggctggacacctggcttcagacttcgccttctcacccccaccaggtgggggtgatgggtcagcagggctggagccgggctgggtggattctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaaccaattgactagtggccttggaggtggatctggtggaggtggatcaggtggaggtggatcaggcggatcaaatcgcacagtctctggtgggcagtatgttgtggctgccgctcccaacttacagaaccagcaagttctgacaggactacctggagtgatgcctaatattcagtatcaagtaatcccacagttccagaccgttgatgggcaacagctgcagtttgctgccactggggcccaagtgcagcaggatggttctggtcaaatacagatcataccaggtgcaaaccaacagattatcacaaatcgaggaagtggaggcaacatcattgctgctatgccaaacctactccagcaggctgtccccctccaaggcctggctaataatgtactctcaggacagactagatct

SEQ ID NO: 87 - DNA, 1437 bp, Artificial Sequence
  Coding sequence for the Fusion protein containing Oct4 and human Ap-2a AD (31-117)
atggactacaaagacgatgacgacaagggatctcaattgttccccatggctggacacctggcttcagacttcgccttctcacccccaccaggtgggggtgatgggtcagcagggctggagccgggctgggtggattctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaaccaattgactagtggccttggaggtggatctggtggaggtggatcaggtggaggtggatcaggcggatcactgggcactgtaggtcaatctccctacacgagcgccccgccgctgtcccacacccccaatgccgacttccagcccccatacttccccccaccctaccagcctatctacccccagtcgcaagatccttactcccacgtcaacgacccctacagcctgaaccccctgcacgcccagccgcagccgcagcacccaggctggcccggccagaggcagagccaggagtctgggctcctgcacacgcaccgggggctgcctcaccagagatct

SEQ ID NO: 88 - DNA, 1773 bp, Artificial Sequence
  Coding sequence for the Fusion protein containing Oct4 and mouse Sox2 AD (121-319)
atggactacaaagacgatgacgacaagggatctcaattgttccccatggctggacacctggcttcagacttcgccttctcacccccaccaggtgggggtgatgggtcagcagggctggagccgggctgggtggattctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaaccaattgactagtggccttggaggtggatctggtggaggtggatcaggtggaggtggatcaggcggatcactcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgagatct

SEQ ID NO: 89 - DNA, 1362 bp, Artificial Sequence
  Coding sequence for the Fusion protein containing Oct4 and mouse Nanog AD (244-305)
atggactacaaagacgatgacgacaagggatctcaattgttccccatggctggacacctggcttcagacttcgccttctcacccccaccaggtgggggtgatgggtcagcagggctggagccgggctgggtggattctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaaccaattgactagtggccttggaggtggatctggtggaggtggatcaggtggaggtggatcaggcggatccaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaataagatct

SEQ ID NO: 90 - DNA, 924 bp, Artificial Sequence
  Coding sequence for the Fusion protein containing Oct4 (127-352) and VP16 AD (446-490)
atggactacaaagacgatgacgacaagggaattggacaattcatgttgggggacggggattccccgggtccgggatttaccccccacgactccgccccctacggcgctctggatatggccgacttcgagtttgagcagatgtttaccgatgcccttggaattgacgagtacggtgggggatctggatcaggtggaggtggatcaggtggaggtggatctggaggtggacttggcagtactgaattcgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaac

SEQ ID NO: 91 - DNA, 1095 bp, Artificial Sequence
  Coding sequence for the Fusion protein containing Oct4 (1-286) and VP16 AD (446-490)
atggactacaaagacgatgacgacaagggatctatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaattgactagtggccttggaggtggatctggtggaggtggatcaggtggaggtggatcaggcatgttgggggacggggattccccgggtccgggatttaccccccacgactccgccccctacggcgctctggatatggccgacttcgagtttgagcagatgtttaccgatgcccttggaattgacgagtacggtgggagatct

SEQ ID NO: 92 - protein, 432 aa, Artificial Sequence
  Fusion protein containing Oct4 and VP16 AD (446-490)
MDYKDDDDKGIGMLGDGDSPGPGFTPHDSAPYGALDMADFEFEQMFTDALGIDEYGGGSGSGGGGSGGGGSGGGLGSTEFMAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 93 - protein, 399 aa, Artificial Sequence
  Fusion protein containing Sox2 and VP16 AD (446-490)
MDYKDDDDKGIGMLGDGDSPGPGFTPHDSAPYGALDMADFEFEQMFTDALGIDEYGGGSGSGGGGSGGGGSGGGLGSTEFMYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHM

SEQ ID NO: 94 - protein, 385 aa, Artificial Sequence
  Fusion protein containing Nanog and VP16 AD (446-490)
MDYKDDDDKGIGMLGDGDSPGPGFTPHDSAPYGALDMADFEFEQMFTDALGIDEYGGGSGSGGGGSGGGGSGGGLGSTELMSVGLPGPHSLPSSEEASNSGNASSMPAVFHPENYSCLQGSATEMLCTEAASPRPSSEDLPLQGSPDSSTSPKQKLSSPEADKGPEEEENKVLARKQKMRTVFSQAQLCALKDRFQKQKYLSLQQMQELSSILNLSYKQVKTWFQNQRMKCKRWQKNQWLKTSNGLIQKGSAPVEYPSIHCSYPQGYLVNASGSLSMWGSQTWTNPTWSSQTWTNPTWNNQTWTNPTWSSQAWTAQSWNGQPWNAAPLHNFGEDFLQPYVQLQQNFSASDLEVNLEATRESHAHFSTPQALELFLNYSVTPPGEI

SEQ ID NO: 95 - protein, 381 aa, Artificial Sequence
  Fusion protein containing Nanog and VP16 AD (446-490)
MSVGLPGPHSLPSSEEASNSGNASSMPAVFHPENYSCLQGSATEMLCTEAASPRPSSEDLPLQGSPDSSTSPKQKLSSPEADKGPEEEENKVLARKQKMRTVFSQAQLCALKDRFQKQKYLSLQQMQELSSILNLSYKQVKTWFQNQRMKCKRWQKNQWLKTSNGLIQKGSAPVEYPSIHCSYPQGYLVNASGSLSMWGSQTWTNPTWSSQTWTNPTWNNQTWTNPTWSSQAWTAQSWNGQPWNAAPLHNFGEDFLQPYVQLQQNFSASDLEVNLEATRESHAHFSTPQALELFLNYSVTPPGEIRSTSGLGGGSGGGGSGGGGSGQFMLGDGDSPGPGFTPHDSAPYGALDMADFEFEQMFTDALGIDEYGGDYKDDDDK

SEQ ID NO: 96 - protein, 531 aa, Artificial Sequence
  Fusion protein containing Oct4 and three VP16 AD (446-490) linked in series
MDYKDDDDKGSQLFPMAGHLASDFAFSPPPGGGDGSAGLEPGWVDSRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 97 - protein, 416 aa, Artificial Sequence
  Fusion protein containing Oct4 and two VP16 AD (437-448) linked in series
MDYKDDDDKGSQLFPMAGHLASDFAFSPPPGGGDGSAGLEPGWVDSRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNQLTSGLGGGSGGGGSGGGGSGGSDALDDFDLDMLGSDALDDFDLDMLGS

SEQ ID NO: 98 - protein, 503 aa, Artificial Sequence
  Fusion protein containing Oct4 and yeast Gal4 AD (768-881)
MDYKDDDDKGSQLFPMAGHLASDFAFSPPPGGGDGSAGLEPGWVDSRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNQLTSGLGGGSGGGGSGGGGSGGSNFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKE

SEQ ID NO: 99 - protein, 495 aa, Artificial Sequence
  Fusion protein containing Oct4 and human NF κ B AD (451-551)
MDYKDDDDKGSQLFPMAGHLASDFAFSPPPGGGDGSAGLEPGWVDSRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNQLTSGLGGGSGGGGSGGGGSGGSGSGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSRS

SEQ ID NO: 100 - protein, 415 aa, Artificial Sequence
  Fusion protein containing Oct4 and mouse p53 TAD (8-32)
MDYKDDDDKGSQLFPMAGHLASDFAFSPPPGGGDGSAGLEPGWVDSRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNQLTSGLGGGSGGGGSGGGGSGGSQSDISLELPLSQETFSGLWKLLPPE

SEQ ID NO: 101 - protein, 504 aa, Artificial Sequence
  Fusion protein containing Oct4 and human Sp1a AD (139-250)
MDYKDDDDKGSQLFPMAGHLASDFAFSPPPGGGDGSAGLEPGWVDSRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNQLTSGLGGGSGGGGSGGGGSGGSNRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTRS

SEQ ID NO: 102 - protein, 479 aa, Artificial Sequence
  Fusion protein containing Oct4 and human Ap-2a AD (31-117)
MDYKDDDDKGSQLFPMAGHLASDFAFSPPPGGGDGSAGLEPGWVDSRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNQLTSGLGGGSGGGGSGGGGSGGSLGTVGQSPYTSAPPLSHTPNADFQPPYFPPPYQPIYPQSQDPYSHVNDPYSLNPLHAQPQPQHPGWPGQRQSQESGLLHTHRGLPHQRS

SEQ ID NO: 103 - protein, 591 aa, Artificial Sequence
  Fusion protein containing Oct4 and mouse Sox2 AD (121-319)
MDYKDDDDKGSQLFPMAGHLASDFAFSPPPGGGDGSAGLEPGWVDSRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNQLTSGLGGGSGGGGSGGGGSGGSLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHMRS

SEQ ID NO: 104 - protein, 454 aa, Artificial Sequence
  Fusion protein containing Oct4 and mouse Nanog AD (244-305)
MDYKDDDDKGSQLFPMAGHLASDFAFSPPPGGGDGSAGLEPGWVDSRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSNQLTSGLGGGSGGGGSGGGGSGGSNAAPLHNFGEDFLQPYVQLQQNFSASDLEVNLEATRESHAHFSTPQALELFLNYSVTPPGEIRS

SEQ ID NO: 105 - protein, 308 aa, Artificial Sequence
  Fusion protein containing Oct4 (127-352) and VP16 AD (446-490)
MDYKDDDDKGIGQFMLGDGDSPGPGFTPHDSAPYGALDMADFEFEQMFTDALGIDEYGGGSGSGGGGSGGGGSGGGLGSTEFEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 106 - protein, 365 aa, Artificial Sequence
  Fusion protein containing Oct4 (1-286) and VP16 AD (446-490)
MDYKDDDDKGSMAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQLTSGLGGGSGGGGSGGGGSGMLGDGDSPGPGFTPHDSAPYGALDMADFEFEQMFTDALGIDEYGGRS

SEQ ID NO: 107 - protein, 280 aa, Artificial Sequence
  Fusion protein of Tcl1 and VP16 AD (446-490)
MATQRAHRAETPAHPNRLWIWEKHVYLDEFRRSWLPVVIKSNEKFQVILRQEDVTLGEAMSPSQLVPYELPLMWQLYPKDRYRSCDSMYWQILYHIKFRDVEDMLLELIDSESNDEQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 108 - protein, 817 aa, Artificial Sequence
  Fusion protein of Tcf3 and VP16 AD (446-490)
MMNQSQRMAPVGSDKELSDLLDFSMMFPLPVANGKSRPASLGGTQFAGSGLEDRPSSGSWGSSDQNSSSFDPSRTYSEGAHFSDSHSSLPPSTFLGAGLGGKGSERNAYATFGRDTSVGTLSQAGFLPGELSLSSPGPLSPSGIKSSSQYYPSFPSNPRRRAADGGLADTQPKKVRKVPPGLPSSVYPPSSGDSYSRDAAAYPSAKTPSSAYPSPFYVADGSLHPSAELWSTPSQVGFGPMLGDGSSPLPLAPGSSSVGSGTFGGLQQQDRMGYQLHGSEVNGSLPAVSSFSAAPGTYSGTSGHTPPVSGAAAESLLGTRGTTASSSGDALGKALASIYSPDHSSNNFSPSPSTPVGSPQGLPGTSQWPRAGAPSALSPNYDAGLHGLQSKMEDRLDEAIHVLRSHAVGTASDLHGLLPGHGALTTSFTGPMSLGGRHAGLVGGSHPEEGLTSGASLLHNHASLPSQPSSLPDLSQRPPDSYSGLGRAGTTAGASEIKREEKEDEEIASVADAEEDKKDLKVPRTRTSPDEDEDDLLPPEQKAEREKERRVANNARERLRVRDINEAFKELGRMCQLHLSSEKPQTKLLILHQAVAVILSLEQQVRERNLNPKAACLKRREEEKVSGVVGDPQLALSAAHPGLGEAHNPAGHLQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 109 - protein, 452 aa, Artificial Sequence
  Fusion protein of Rex1 and VP16 AD (446-490)
MNEQKMNEQMKKTAKTSGQKGPGGRALDRLTLKQDEARPVQNTRVEAPRVTYTIRDESEISPETEEDGFPDGYLECIIRGEFSEPILEEDFLFKSFESLEEVEQNLSRQVLEASSLLESSLEYMTKGTKQEKREVTQETPPLRVGASSLLAGGPAEKPEGGVYCGVLSMLECPQAGCKKKLRDKTALRKHMLVHGPRRHVCAECGKAFTESSKLKRHFLVHTGEKPYQCTFEGCGKRFSLDFNLRTHIRIHTGERRFVCPFDGCEKSFIQSNNQKIHILTHAKAGKKCQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 110 - protein, 1231 aa, Artificial Sequence
  Fusion protein of Sal4 and VP16 AD (446-490)
MSRRKQAKPQHINWEEGQGEQPQQLPSPDLAEALAAEEPGAPVNSPGNCDEASEDSIPVKRPRREDTHICNKCCAEFFSLSEFMEHKKSCTKTPPVLIMNDSEGPVPSEDFSRAALSHQLGSPSNKDSLQENGSSSGDLKKLGTDSILYLKTEATQPSTPQDISYLPKGKVANTNVTLQALRGTKVAVNQRGAEAPMAPMPAAQGIPWVLEQILCLQQQQLQQIQLTEQIRVQVNMWAAHALHSGVAGADTLKALSSHVSQQVSVSQQVSAAVALLSQKASNPALSLDALKQAKLPHASVPSAASPLSSGLTSFTLKPDGTRVLPNFVSRLPSALLPQTPGSVLLQSPFSAVTLDQSKKGKGKPQNLSASASVLDVKAKDEVVLGKHKCRYCPKVFGTDSSLQIHLRSHTGERPYVCPICGHRFTTKGNLKVHLQRHPEVKANPQLLAEFQDKGAVSAASHYALPVPVPADESSLSVDAEPVPVTGTPSLGLPQKLTSGPNSRDLMGGSLPNDMQPGPSPESEAGLPLLGVGMIHNPPKAGGFQGTGAPESGSETLKLQQLVENIDKATTDPNECLICHRVLSCQSSLKMHYRTHTGERPFQCKICGRAFSTKGNLKTHLGVHRTNTTVKTQHSCPICQKKFTNAVMLQQHIRMHMGGQIPNTPLPESPCDFTAPEPVAVSENGSASGVCQDDAAEGMEAEEVCSQDVPSGPSTVSLPVPSAHLASPSLGFSVLASLDTQGKGALPALALQRQSSRENSSLEGGDTGPANDSSLLVGDQECQSRSPDATETMCYQAVSPANSQAGSVKSRSPEGHKAEGVESCRVDTEGRTSLPPTFIRAQPTFVKVEVPGTFVGPPSMPSGMPPLLASQPQPRRQAKQHCCTRCGKNFSSASALQIHERTHTGEKPFVCNICGRAFTTKGNLKVHYMTHGANNNSARRGRKLAIENPMAALSAEGKRAPEVFSKELLSPAVSVDPASWNQYTSVLNGGLAMKTNEISVIQSGGIPTLPVSLGASSVVSNGTISKLDGSQTGVSMPMSGNGEKLAVPDGMAKHQFPHFLEENKIAVSQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 111 - protein, 532 aa, Artificial Sequence
  Fusion protein of lefty1 and VP16 AD (446-490)
MPFLWLCWALWALSLVSLREALTGEQILGSLLQQLQLDQPPVLDKADVEGMVIPSHVRTQYVALLQHSHASRSRGKRFSQNLREVAGRFLVSETSTHLLVFGMEQRLPPNSELVQAVLRLFQEPVPRTALRRQKRLSPHSARARVTIEWLRFRDDGSNRTALIDSRLVSIHESGWKAFDVTEAVNFWQQLSRPRQPLLLQVSVQREHLGPGTWSSHKLVRFAAQGTPDGKGQGEPQLELHTLDLKDYGAQGNCDPEAPVTEGTRCCRQEMYLDLQGMKWAENWILEPPGFLTYECVGSCLQLPESLTSRWPFLGPRQCVASEMTSLPMIVSVKEGGRTRPQVVSLPNMRVQTCSCASDGALIPRRLQPQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 112 - protein, 465 aa, Artificial Sequence
  Fusion protein of Dppa2 and VP16 AD (446-490)
MSYFGLETFNENQSEENLDEESVILTLVPFKEEEEPNTDYATQSNVSSSTLDHTPPARSLVRHAGIKHPTRTIPSTCPPPSLPPIRDVSRNTLREWCRYHNLSTDGKKVEVYLRLRRHSYSKQECYIPNTSREARMKQGPKKSKIVFRGIGPPSGCQRKKEESGVLEILTSPKESTFAAWARIAMRAAQSMSKNRCPLPSNVEAFLPQATGSRWCVVHGRQLPADKKGWVRLQFLAGQTWVPDTPQRMNFLFLLPACIIPEPGVEDNLLCPECVHSNKKILRNFKIRSRAKKNALPPNMPPQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 113 - protein, 460 aa, Artificial Sequence
  Fusion protein of Dppa4 and VP16 AD (446-490)
METAGDKKWSAEEPKEEVELQMSSQPSTAPAKAKATGKKQKKSETDNGCKPKEGKPQDTETPGQTRRKVPIPPIPEYLPPVNLIHRDVLRAWCQKKRVSSKGQKLDAYKRLLARAFPEQMLELRNVPDSAKDARLKTAHKKMKTEPGEESEVTVPLEMVPVPEEQIPALIDPPMLYEEVSTTVVTTPATEAVLASWARIASNAKKYEAVPADASSSSEVKGEMWCVVHGTSLPGNSRGWVRLQFHAGQAWVPDKKGKAIALFLLPACTFPPPHLEDNMLCPKCVHKNKILTKSLEGQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 114 - protein, 282 aa, Artificial Sequence
  Fusion protein of Dppa5 and VP16 AD (446-490)
MMVTLVTRKDIPPWVKVPEDLKDPEVFQVQSLVLKYLFGPQGSRMSHIEQVSQAMFELKNLESPEELIEVFIYGSQNNKIRAKWMLQSMAERYHLRQQKGVLKLEESMKTLELGQCIEQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 115 - protein, 626 aa, Artificial Sequence
  Fusion protein of Nr5a1 and VP16 AD (446-490)
MDYSYDEDLDELCPVCGDKVSGYHYGLLTCESCKGFFKRTVQNNKHYTCTESQSCKIDKTQRKRCPFCRFQKCLTVGMRLEAVRADRMRGGRNKFGPMYKRDRALKQQKKAQIRANGFKLETGPPMGVPPPPPPPPDYMLPPSLHAPEPKALVSGPPSGPLGDFGAPSLPMAVPGPHGPLAGYLYPAFSNRTIKSEYPEPYASPPQQPGPPYSYPEPFSGGPNVPELILQLLQLEPEEDQVRARIVGCLQEPAKSRSDQPAPFSLLCRMADQTFISIVDWARRCMVFKELEVADQMTLLQNCWSELLVLDHIYRQVQYGKEDSILLVTGQEVELSTVAVQAGSLLHSLVLRAQELVLQLHALQLDRQEFVCLKFLILFSLDVKFLNNHSLVKDAQEKANAALLDYTLCHYPHCGDKFQQLLLCLVEVRALSMQAKEYLYHKHLGNEMPRNNLLIEMLQAKQTQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 116 - protein, 724 aa, Artificial Sequence
  Fusion protein of Nr5a2 and VP16 AD (446-490)
MSASLDTGDFQEFLKHGLTAIASAPGSETRHSPKREEQLREKRAGLPDRHRRPIPARSRLVMLPKVETEAPGLVRSHGEQGQMPENMQVSQFKMVNYSYDEDLEELCPVCGDKVSGYHYGLLTCESCKGFFKRTVQNQKRYTCIENQNCQIDKTQRKRCPYCRFKKCIDVGMKLEAVRADRMRGGRNKFGPMYKRDRALKQQKKALIRANGLKLEAMSQVIQAMPSDLTSAIQNIHSASKGLPLSHVALPPTDYDRSPFVTSPISMTMPPHSSLHGYQPYGHFPSRAIKSEYPDPYSSSPESMMGYSYMDGYQTNSPASIPHLILELLKCEPDEPQVQAKIMAYLQQEQSNRNRQEKLSAFGLLCKMADQTLFSIVEWARSSIFFRELKVDDQMKLLQNCWSELLILDHIYRQVAHGKEGTIFLVTGEHVDYSTIISHTEVAFNNLLSLAQELVVRLRSLQFDQREFVCLKFLVLFSSDVKNLENLQLVEGVQEQVNAALLDYTVCNYPQQTEKFGQLLLRLPEIRAISKQAEDYLYYKHVNGDVPYNNLLIEMLHAKRAQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 117 - protein, 636 aa, Artificial Sequence
  Fusion protein of Dax1 and VP16 AD (446-490)
MAGEDHPWQGSILYNLLMSAKQKHASQEEREVRLGAQCWGCACGAQPVLGGERLSGGQARSLLYRCCFCGENHPRQGGILYSMLTNARQPSVATQAPRARFGAPCWGCACGSAEPLVGREGLPAGQAPSLLYRCCFCGEEHPRQGSILYSLLTSAQQTHVSREAPEAHRRGEWWQLSYCTQSVGGPEGLQSTQAMAFLYRSYVCGEEQPQQISVASGTPVSADQTPATPQEQPRAPWWDASPGVQRLITLKDPQVVCEAASAGLLKTLRFVKYLPCFQILPLDQQLVLVRSCWAPLLMLELAQDHLHFEMMEIPETNTTQEMLTTRRQETEGPEPAEPQATEQPQMVSAEAGHLLPAAAVQAIKSFFFKCWSLNIDTKEYAYLKGTVLFNPDLPGLQCVKYIEGLQWRTQQILTEHIRMMQREYQIRSAELNSALFLLRFINSDVVTELFFRPIIGAVSMDDMMLEMLCAKLQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 118 - protein, 618 aa, Artificial Sequence
  Fusion protein of Esrrb and VP16 AD (446-490)
MDVSELCIPDPLGYHNQLLNRMSSEDRHLGSSCGSFIKTEPSSPSSGIDALSHHSPSGSSDASGGFGIALSTHANGLDSPPMFAGAGLGGNPCRKSYEDCTSGIMEDSAIKCEYMLNAIPKRLCLVCGDIASGYHYGVASCEACKAFFKRTIQGNIEYNCPATNECEITKRRRKSCQACRFMKCLKVGMLKEGVRLDRVRGGRQKYKRRLDSENSPYLNLPISPPAKKPLTKIVSNLLGVEQDKLYAMPPNDIPEGDIKALTTLCELADRELVFLINWAKHIPGFPSLTLGDQMSLLQSAWMEILILGIVYRSLPYDDKLAYAEDYIMDEEHSRLVGLLDLYRAILQLVRRYKKLKVEKEEFMILKALALANSDSMYIENLEAVQKLQDLLHEALQDYELSQRHEEPRRAGKLLLTLPLLRQTAAKAVQHFYSVKLQGKVPMHKLFLEMLEAKVQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 119 - protein, 503 aa, Artificial Sequence
  Fusion protein of Utf1 and VP16 AD (446-490)
MLLRPRRLPAFSPPSPASPDAELRSAGDVPVTTSDAFATSGGMAEPGSPKAPVSPDSAQRTPWSARETELLLGTLLQPAMWRSLLLDRRQTLPTYRRVSAALARQQVRRTPAQCRRRYKFLKDKLRDSQGQPSGPFDNQIRQLMGLLGDDGPPRVRRRSTGPGRPQRRGRSSLSALAPAPAPVEQEAELPLAAENDEPAPALRFSSSTTKSAGAHRITSSPPLTSTDTLPPEPGHTFESSPTPTPDHDVETPNEPPGLSQGRASSPQVAPQSLNTALLQTLTHLGDISTVLGPLRDQLSTLNQHVEHLRGSFDQTVSLAVGFILGSAASERGILGDLRQQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 120 - protein, 905 aa, Artificial Sequence
  Fusion protein of Tbx3 and VP16 AD (446-490)
MSLSMRDPVIPGTSMAYHPFLPHRAPDFAMSAVLGHQPPFFPALTLPPNGAAALSLPGALAKPIMDQLVGAAETGIPFSSLGPQAHLRPLKTMEPEEDVEDDPKVHLEAKELWDQFHKRGTEMVITKSGRRMFPPFKVRCSGLDKKAKYILLMDIIAADDCRYKFHNSRWMVAGKADPEMPKRMYIHPDSPATGEQWMSKVVTFHKLKLTNNISDKHGFTLAFPSDHATWQGNYSFGTQTILNSMHKYQPRFHIVRANDILKLPYSTFRTYLFPETEFIAVTAYQNDKITQLKIDNNPFAKGFRDTGNGRREKRKQLTLQSMRVFEERHKKETSDESSSEQAAFNCFAQASSPAVSIVGTSNLKDLCPSEAESDAEAESKEEHGPEACDAAKISTTTAEEPGRDKGSPATRAQLFPAEPSRARDTARLDKASPDSRHSPATISSSTRVPGADERRSPGREGPVATKVDEARAIPAKDAFAPLSVQTDATAHLAQGPLPGLGFAPGLAGQQFFNGHPLFLHPGQFAMGGAFSSMAAGMGPLLATVSGASTGVSGLESTAMASAAAAQGLSGASAATLPFHLQQHVLASQGLAMSPFGSLFPYPYTYMAAAAAASTAAASSSVHRHPFLNLNSMRPRLRYSPYSIPVPVPDSSSLLATALPSMASAAGPLDGKAAALAASPASVAVDSGSELNSRSSTLSSGSVSLSPKLCSEKEAATSELQSIQRLVSGLEAKPDRSCSGSPQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 121 - protein, 381 aa, Artificial Sequence
  Fusion protein of Grb2 and VP16 AD (446-490)
MEAIAKYDFKATADDELSFKRGDILKVLNEECDQNWYKAELNGKDGFIPKNYIEMKPHPWFFGKIPRAKAEEMLSKQRHDGAFLIRESESAPGDFSLSVKFGNDVQHFKVLRDGAGKYFLWVVKFNSLNELVDYHRSTSVSRNQQIFLRDIEQMPQQPTYVQALFDFDPQEDGELGFRRGDFIHVMDNSDPNWWKGACHGQTGMFPRNYVTPVNRNVQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 122 - protein, 3230 aa, Artificial Sequence
  Fusion protein of Tel1 and VP16 AD (446-490)
MSLALNDLLICCRQLEHDRATERRKEVDKFKRLIQDPETVQHLDRHSDSKQGKYLNWDAVFRFLQKYIQKEMESLRTAKSNVSATTQSSRQKKMQEISSLVRYFIKCANKRAPRLKCQDLLNYVMDTVKDSSNGLTYGADCSNILLKDILSVRKYWCEVSQQQWLELFSLYFRLYLKPSQDINRVLVARIIHAVTRGCCSQTDGLPSKFLDLFSKAIQYARQEKSSPGLSHILAALNIFLKSLAVNFRKRVCEAGDEILPTLLYIWTQHRLNDSLKEVIIELIQLQIYIHHPQGARAPEEGAYESMKWKSILYNLYDLLVNEISHIGSRGKYSSGSRNIAVKENLIDLMADICYQLFDADTRSVEISQSYVTQRESTDYSVPCKRRKIDVGWEVIKDYLQKSQSDFDLVPWLQITTRLISKYPSSLPNCELSPLILILYQLLPQQRRGERIPYVLRCLKEVALCQGKKSNLESSQKSDLLKLWIKIWSITFRGISSGQTQTENFGLLEAIIQGSLVELDREFWKLFTGSACKPSSPSVCCLTLALSICVVPDAIKMGTEQSVCEANRSFSVKESIMRWLLFYQLEDDLEDSTELPPILQSNFPHLVVEKILVSLTMKNSKAAMKFFQSVPECEQHCEDKEEPSFSEVEELFLQTTFDKMDFLTTVKEYAVEKFQSSVGFSVQQNLKESLDHYLLGLSEQLLSNYSSEITSSETLVRCSSLLVGVLGCYCYMGIITEDEAHKSELFQKAKSLMQCAGESISLFKNKTNEESRIGSLRNVMHLCTSCLCIHTKHTPNKIASGFFLRLLTSKLMNDIADICKSLASCTKKPLDHGVHPGEDDEDGGGCDSLMEAEGPSSTGLSTAYPASSVSDANDYGENQNAVGAMSPLAADYLSKQDHLLLDMLRFLGRSVTASQSHTVSFRGADIRRKLLLLLDSSILDLMKPLHLHMYLVLLKDLPGNEHSLPMEDVVELLQPLSLVCSLHRRDQDVCKTILSNVLHIVTNLGQGSVDMESTRIAQGHFLTVMGAFWHLTKEKKCVFSVRMALVKCLQTLLEADPYSEWAILNVKGQDFPVNEAFSQFLADDHHQVRMLAAGSVNRLFQDMRQGDFSRSLKALPLKFQQTSFNNAYTTAEAGIRGLLCDSQNPDLLDEIYNRKSVLLMMIAVVLHCSPVCEKQALFALCKSVKENRLEPHLVKKVLEKVSESFGCRSLEDFMISHLDYLVLEWLNLQDTEYSLSSFPFMLLNYTSIEDFYRSCYKILIPHLVIRSHFDEVKSIANQIQKCWKSLLVDCFPKILVHILPYFAYEGTRDSYVSQKRETATKVYDTLKGEDFLGKQIDQVFISNLPEIVVELLMTLHETADSADSDASQSATALCDFSGDLDPAPNPPYFPSHVIQATFAYISNCHKTKFKSILEILSKIPDSYQKILLAICEQAAETNNVFKKHRILKIYHLFVSLLLKDIQSGLGGAWAFVLRDVIYTLIHYINKRSSHFTDVSLRSFSLCCDLLSRVCHTAVTQCKDALESHLHVIVGTLIPLVDYQEVQEQVLDLLKYLVIDNKDNKNLSVTIKLLDPFPDHVIFKDLRLTQQKIKYSGGPFSLLEEINHFLSVSAYNPLPLTRLEGLKDLRRQLEQHKDQMLDLLRASQDNPQDGIVVKLVVSLLQLSKMAVNQTGEREVLEAVGRCLGEIGPLDFSTIAVQHNKDVSYTKAYGLPEDRELQWTLIMLTALNNTLVEDSVKIRSAAATCLKNILATKIGHIFWENYKTSADPMLTYLQPFRTSRKKFLEVPRSVKEDVLEGLDAVNLWVPQSESHDIWIKTLTCAFLDSGGINSEILQLLKPMCEVKTDFCQMLLPYLIHDVLLQDTHESWRTLLSAHVRGFFTSCFKHSSQASRSATPANSDSESENFLRCCLDKKSQRTMLAVVDYLRRQKRPSSGTAFDDAFWLDLNYLEVAKVAQSCSAHFTALLYAEIYSDKKSTDEQEKRSPTFEEGSQGTTISSLSEKSKEETGISLQDLLLEIYRSIGEPDSLYGCGGGKMLQPLTRIRTYEHEATWEKALVTYDLETSISSSTRQSGIIQALQNLGLSHILSVYLKGLDYERREWCAELQELRYQAAWRNMQWGLCASAGQEVEGTSYHESLYNALQCLRNREFSTFYESLRYASLFRVKEVEELSKGSLESVYSLYPTLSRLQAIGELENSGELFSRSVTDRERSEAYWKWQKHSQLLKDSDFSFQEPLMALRTVILETLVQKEMERSQGACSKDILTKHLVEFSVLARTFKNTQLPERAIFKIKQYNSAICGISEWHLEEAQVFWAKKEQSLALSILKQMIKKLDSSFKDKENDAGLKVIYAECLRVCGSWLAETCLENPAVIMQTYLEKAVKVAGSYDGNSRELRNGQMKAFLSLARFSDTQYQRIENYMKSSEFENKQTLLKRAKEEVGLLREHKIQTNRYTVKVQRELELDECALRALREDRKRFLCKAVENYINCLLSGEEHDLWVFRLCSLWLENSGVSEVNGMMKKDGMKISSYKFLPLMYQLAARMGTKMTGGLGFHEVLNNLISRISLDHPHHTLFIILALANANKDEFLSKPETTRRSRITKSTSKENSHLDEDRTEAATRIIHSIRSKRCKMVKDMEALCDAYIILANMDASQWRAQRKGINIPANQPITKLKNLEDVVVPTMEIKVDPTGEYENLVTIKSFKTEFRLAGGLNLPKIIDCVGSDGKERRQLVKGRDDLRQDAVMQQVFQMCNTLLQRNTETRKRKLTICTYKVVPLSQRSGVLEWCTGTVPIGEYLVNSEDGAHRRYRPNDFSANQCQKKMMEVQKKSFEEKYDTFMTICQNFEPVFRYFCMEKFLDPAVWFEKRLAYTRSVATSSIVGYILGLGDRHVQNILINEQSAELVHIDLGVAFEQGKILPTPETVPFRLSRDIVDGMGITGVEGVFRRCCEKTMEVMRSSQETLLTIVEVLLYDPLFDWTMNPLKALYLQQRPEDESDLHSTPNADDQECKQSLSDTDQSFNKVAERVLMRLQEKLKGVEEGTVLSVGGQVNLLIQQAMDPKNLSRLFPGWKAWVQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 123 - protein, 395 aa, Artificial Sequence
  Fusion protein of Sox15 and VP16 AD (446-490)
MALTSSSQAETWSLHPRASTASLPLGPQEQEAGGSPGASGGLPLEKVKRPMNAFMVWSSVQRRQMAQQNPKMHNSEISKRLGAQWKLLGDEEKRPFVEEAKRLRARHLRDYPDYKYRPRRKSKNSSTGSVPFSQEGGGLACGGSHWGPGYTTTQGSRGFGYQPPNYSTAYLPGSYTSSHCRPEAPLPCTFPQSDPRLQGELRPSFSPYLSPDSSTPYNTSLAGAPMPVTHLQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 124 - protein, 530 aa, Artificial Sequence
  Fusion protein of Gdf3 and VP16 AD (446-490)
MQPYQRLLALGFLLLTLPWGQTSEFQDSDLLQFLGLEKAPSPHRFQPVPRVLRKIIRAREAAAASGASQDLCYVKELGVRGNLLQLLPDQGFFLNTQKPFQDGSCLQKVLYFNLSAIKEKAKLTMAQLTLDLGPRSYYNLRPELVVALSVVQDRGVWGRSHPKVGRLLFLRSVPGPQGQLQFNLQGALKDWSSNRLKNLDLHLEILVKEDRYSRVTVQPENPCDRLLRSLHASLLVVTLNPKHCHPSSRKRRAAISVPKGFCRNFCHRHQLFINFQDLGWHKWVIAPKGFMANYCHGECPFSMTTYLNSSNYAFMQALMHMADPKVPKAVCVPTKLSPISMLYQDSDKNVILRHYEDMVVDECGCGQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 125 - protein, 381 aa, Artificial Sequence
  Fusion protein of Ecat1 and VP16 AD (446-490)
MDAPRRFPTLVQLMQPKAMPVEVLGHLPKRFSWFHSEFLKNPKVVRLEVWLVEKIFGRGGERIPHVQGMSQILIHVNRLDPNGEAEILVFGRPSYQEDTIKMIMNLADYHRQLQAKGSGKALAQDVATQKAETQRSSIEVREAGTQRSVEVREAGTQRSVEVQEVGTQGSPVEVQEAGTQQSLQAANKSGTQRSPEAASKAVTQRFREDARDPVTRLQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 126 - protein, 571 aa, Artificial Sequence
  Fusion protein of Ecat8 and VP16 AD (446-490)
MFEVLVLKIEDPGCFWVIIKGCSHFLEQEVDYQKLNTAMNDFYNSMCQDVEMKPLMLEEGQVCVVYCQELKCWCRALIKSIISSADHYLAECFLVDFAKYIPVKSKNIRVAVESFMQLPYRAKKFRLYGTKPVTLHIDFCEDNAEIVPATKWDSAAIQYFQNLLRATTQVEAKLCAVEEDTFEVYLYATIKNEKVCVNDDLVAKNFAYYVSPMGNKNLNPLEKPRQSLNSVTCSSKLSPSLTLWPMLLQGKDYHRMENKALNYKDSLTDSPKMMLEKQQQSLPLKHTEKCTESSVYWPTKRGITIYADPDVPSVSGSSQRPNEKPLRLTEKKDCDEKNGCVKLLQFLNPDPLRADGTSDLHQLQKVKLGTLQPGVVLRNRIEPCLTLEKSPLSADLKKVNMFLKPDSQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 127 - protein, 642 aa, Artificial Sequence
  Fusion protein of Fbxo15 and VP16 AD (446-490)
MEESELEIFRSKFVRGSSVTKQHAWRNQHSEKRCSSSISSISLDRMPSEILVKILSYLDAVTLVCIGCVSRRFYHLADDNLIWVRKYAAAFRSKRSRWKATSVEETATSLSLLSVWDKEDGYWKKEYITKQISSVKAALTNSLSPVKRRTSLPSKTKESLRISGLGWTIILREASGKEHIMQHSNLSVNDNSVTVFWHDKNWPHVDTLSTLDLYGATPIFMEQYKGPNTSCPRWLSLIEKYDLSNLRKSAMIGCDRHVRVFCVNPGLLVGLWQENGGLAFVMANIHSHGLFERSIMGSDTIPYTLPPDTTFVDNYPDSMTFYGDKGFQLHIDIHGSKTYFLCSTFHNLFCRRAGINNGYVKFLMINLKNNREHLPLVGKVGLEWRTDCLNGRIESCIVVDMTLLDEDKKPIWYVSSPVCLRSACLPDFPQPAYSFEYMDSVGGVCADLGWFENTDEYFIVRLDIYLSVAKLQQWFGRQQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 128 - protein, 391 aa, Artificial Sequence
  Fusion protein of eRas and VP16 AD (446-490)
MALPTKSSILDLSSGTPCTRSPEESHEAWAQCKDAGRQLPEYKAVVVGASGVGKSALTIQMTHQCFVKDHDPTIQDSYWKEVARDNGGYILNVLDTSGQDIHRALRDQCLASGDGVLGVFALDDPSSLDQLQQIWSTWTPHHKQPLVLVGNKCDLVTTAGDAHAAAALLAHKLGAPLVKTSAKTRQGVEEAFALLVHEIQRAQEAVAESSKKTRHQKAVCSCGCSVAQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 129 - protein, 633 aa, Artificial Sequence
  Fusion protein of Foxd3 and VP16 AD (446-490)
MTLSGSGSASDMSGQTVLTAEDVDIDVVGEGDDGLEEKDSDAGCDSPAGPPDLRLDEADEGPPVSAHHGQSQPQALALPTEATGPGNDTGAPEADGCKGGEDAVTGGGGPGAGSGATGGLTPNKPKNSLVKPPYSYIALITMAILQSPQKKLTLSGICEFISNRFPYYREKFPAWQNSIRHNLSLNDCFVKIPREPGNPGKGNYWTLDPQSEDMFDNGSFLRRRKRFKRHQQEHLREQTALMMQSFGAYSLAAAAGAGPYGRPYGLHPAAAAGAYSHPAAAAAAAAAAALQYPYALPPVAPVLPPAVPLLPSGELGRKAAAFGSQLGPSLQLQLNTLGAAAAAAGTAGAAGTTSLIKSEPSARPSFSIENIIGAGSAAPGGSAGGGGSGGGAGGGGGSGGGGGAQSFLRPPGTVQSAALMATHQPLSLSRTTATIAPILSVPLSGQFLQPAASAAAAAAAAVQAKWPAQQLTSGLGGGSGGGGSGGGGSGGSMLGDGDSPGPGFTPHDSAPYGALDMADFEFEQMFTDALGIDEYGGRSMLGDGDSPGPGFTPHDSAPYGALDMADFEFEQMFTDALGIDEYGGRSMLGDGDSPGPGFTPHDSAPYGALDMADFEFEQMFTDALGIDEYGGRS

SEQ ID NO: 130 - protein, 490 aa, human herpesvirus 1
MDLLVDELFADMNADGASPPPPRPAGGPKNTPAAPPLYATGRLSQAQLMPSPPMPVPPAALFNRLLDDLGFSAGPALCTMLDTWNEDLFSALPTNADLYRECKFLSTLPSDVVEWGDAYVPERTQIDIRAHGDVAFPTLPATRDGLGLYYEALSRFFHAELRAREESYRTVLANFCSALYRYLRASVRQLHRQAHMRGRDRDLGEMLRATIADRYYRETARLARVLFLHLYLFLTREILWAAYAEQMMRPDLFDCLCCDLESWRQLAGLFQPFMFVNGALTVRGVPIEARRLRELNHIREHLNLPLVRSAATEEPGAPLTTPPTLHGNQARASGYFMVLIRAKLDSYSSFTTSPSEAVMREHAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 131 - protein, 881 aa, Saccharomyces cerevisiae
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSIDSAAHHDNSTIPLDFMPRDALHGFDWSEEDDMSDGLPFLKTDPNNNGFFGDGSLLCILRSIGFKPENYTNSNVNRLPTMITDRYTLASRSTTSRLLQSYLNNFHPYCPIVHSPTLMMLYNNQIEIASKDQWQILFNCILAIGAWCIEGESTDIDVFYYQNAKSHLTSKVFESGSIILVTALHLLSRYTQWRQKTNTSYNFHSFSIRMAISLGLNRDLPSSFSDSSILEQRRRIWWSVYSWEIQLSLLYGRSIQLSQNTISFPSSVDDVQRTTTGPTIYHGIIETARLLQVFTKIYELDKTVTAEKSPICAKKCLMICNEIEEVSRQAPKFLQMDISTTALTNLLKEHPWLSFTRFELKWKQLSLIIYVLRDFFTNFTQKKSQLEQDQNDHQSYEVKRCSIMLSDAAQRTVMSVSSYMDNHNVTPYFAWNCSYYLFNAVLVPIKTLLSNSKSNAENNETAQLLQQINTVLMLLKKLATFKIQTCEKYIQVLEEVCAPFLLSQCAIPLPHISYNNSNGSAIKNIVGSATIAQYPTLPEENVNNISVKYVSPGSVGPSPVPLKSGASFSDLVKLLSNRPPSRNSPVTIPRSTPSHRSVTPFLGQQQQLQSLVPLTPSALFGGANFNQSGNIADSSLSFTFTNSSNGPNLITTQTNSQALSQPIASSNVHDNFMNNEITASKIDDGNNSKPLSPGWTDQTAYNAFGITTGMFNTTTMDDVYNYLFDDEDTPPNPKKE

SEQ ID NO: 132 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 133 - protein, 390 aa, Mus musculus
MTAMEESQSDISLELPLSQETFSGLWKLLPPEDILPSPHCMDDLLLPQDVEEFFEGPSEALRVSGAPAAQDPVTETPGPVAPAPATPWPLSSFVPSQKTYQGNYGFHLGFLQSGTAKSVMCTYSPPLNKLFCQLAKTCPVQLWVSATPPAGSRVRAMAIYKKSQHMTEVVRRCPHHERCSDGDGLAPPQHLIRVEGNLYPEYLEDRQTFRHSVVVPYEPPEAGSEYTTIHYKYMCNSSCMGGMNRRPILTIITLEDSSGNLLGRDSFEVRVCACPGRDRRTEEENFRKKEVLCPELPPGSAKRALPTCTSASPPQKKKPLDGEYFTLKIRGRKRFEMFRELNEALELKDAHATEESGDSRAHSSYLKTKKGQSTSRHKKTMVKKVGPDSD

SEQ ID NO: 134 - protein, 785 aa, Homo sapiens
MSDQDHSMDEMTAVVKIEKGVGGNNGGNGNGGGAFSQARSSSTGSSSSTGGGGQESQPSPLALLAATCSRIESPNENSNNSQGPSQSGGTGELDLTATQLSQGANGWQIISSSSGATPTSKEQSGSSTNGSNGSESSKNRTVSGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYVTNVPVALNGNITLLPVNSVSAATLTPSSQAVTISSSGSQESGSQPVTSGTTISSASLVSSQASSSSFFTNANSYSTTTTTSNMGIMNFTTSGSSGTNSQGQTPQRVSGLQGSDALNIQQNQTSGGSLQAGQQKEGEQNQQTQQQQILIQPQLVQGGQALQALQAAPLSGQTFTTQAISQETLQNLQLQAVPNSGPIIIRTPTVGPNGQVSWQTLQLQNLQVQNPQAQTITLAPMQGVSLGQTSSSNTTLTPIASAASIPAGTVTVNAAQLSSMPGLQTINLSALGTSGIQVHPIQGLPLAIANAPGDHGAQLGLHGAGGDGIHDDTAGGEEGENSPDAQPQAGRRTRREACTCPYCKDSEGRGSGDPGKKKQHICHIQGCGKVYGKTSHLRAHLRWHTGERPFMCTWSYCGKRFTRSDELQRHKRTHTGEKKFACPECPKRFMRSDHLSKHIKTHQNKKGGPGVALSVGTLPLDSGAGSEGSGTATPSALITTNMVAMEAICPEGIARLANSGINVMQVADLQSINISGNGF

SEQ ID NO: 135 - protein, 437 aa, Homo sapiens
MLWKLTDNIKYEDCEDRHDGTSNGTARLPQLGTVGQSPYTSAPPLSHTPNADFQPPYFPPPYQPIYPQSQDPYSHVNDPYSLNPLHAQPQPQHPGWPGQRQSQESGLLHTHRGLPHQLSGLDPRRDYRRHEDLLHGPHALSSGLGDLSIHSLPHAIEEVPHVEDPGINIPDQTVIKKGPVSLSKSNSNAVSAIPINKDNLFGGVVNPNEVFCSVPGRLSLLSSTSKYKVTVAEVQRRLSPPECLNASLLGGVLRRAKSKNGGRSLREKLDKIGLNLPAGRRKAANVTLLTSLVEGEAVHLARDFGYVCETEFPAKAVAEFLNRQHSDPNEQVTRKNMLLATKQICKEFTDLLAQDRSPLGNSRPNPILEPGIQSCLTHFNLISHGFGSPAVCAAVTALQNYLTEALKAMDKMYLSNNPNSHTDNNAKSSDKEEKHRK

SEQ ID NO: 136 - protein, 319 aa, Mus musculus
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHM

SEQ ID NO: 137 - protein, 305 aa, Mus musculus
MSVGLPGPHSLPSSEEASNSGNASSMPAVFHPENYSCLQGSATEMLCTEAASPRPSSEDLPLQGSPDSSTSPKQKLSSPEADKGPEEEENKVLARKQKMRTVFSQAQLCALKDRFQKQKYLSLQQMQELSSILNLSYKQVKTWFQNQRMKCKRWQKNQWLKTSNGLIQKGSAPVEYPSIHCSYPQGYLVNASGSLSMWGSQTWTNPTWSSQTWTNPTWNNQTWTNPTWSSQAWTAQSWNGQPWNAAPLHNFGEDFLQPYVQLQQNFSASDLEVNLEATRESHAHFSTPQALELFLNYSVTPPGEI